(19) United States
(12) Patent Application Publication
(10) Pub. No.: US 2010/0279278 A1
(43) Pub. Date: Nov. 4, 2010

(54) Methods of detecting one or more bioterrorism target agents

(75) Inventor: Marc R. Labgold, Reston, VA (US)

Correspondence Address: Steven B. Kelber;Berenato & White, LLC, 6550 Rock Spring Drive, Suite 240, Bethesda, MD, 20817, US

(73) Assignee: WAIMANA ENTERPRISES. INC, Honolulu, HI (US)

(21) Appl. No.: 11/998,615

(22) Filed: Nov. 30, 2007

Related U.S. Application Data
(60) Provisional application No. 60/861,818, filed on Nov. 30, 2006.

(51) Int. Cl. C12Q 1/68

(52) U.S. Cl. 435/6

(57) ABSTRACT

The present invention provides a methods and compositions for early diagnosis of exposure to or infection by a chemical or biological weapon by rapid and specific detection of one or more bioterrorism target agents in a sample.

### FIELD OF THE INVENTION

This invention relates to methods and compositions capable of rapid diagnosis of exposure to or infection by biological or chemical weapons as well as kits for performing such diagnosis.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Enzyme-linked immunosorbent assay (ELISA) is a widely used method for measuring the concentration of a particular molecule (e.g., a hormone or drug) in a fluid such as serum or urine. It is also known as enzyme immunoassay or EIA. The molecule or target agent is detected by antibodies that have been made against it; that is, for which it is the antigen. Monoclonal antibodies are often used. Due to the diversity found in the immune system and the production of monoclonal antibodies from immortalized cells of the immune system, first described by Kohler and Milstein in 1975, antibodies can be raised against a huge number of different antigens by standard immunological techniques. Potentially any target agent can be recognized by a specific antibody that will not react with any other target agent.

An ELISA typically involves coating a vessel, such as a microtiter plate with an antibody specific for a particular antigen to be detected, e.g., a molecule derived from a virus or bacteria, adding the sample suspected of containing the particular antigen, allowing the antibody to bind the antigen and then adding at least one other antibody specific to another region of the same antigen to be detected. This use of two antibodies can be referred to as a “sandwich” ELISA. Sometimes, the second antibody or even a third antibody is used that is labeled with a chromogenic or fluorogenic reporter molecule to aid in detection. The procedure may also involve the need for a chemical substrate to produce a signal. The need for multiple antibodies, which do not cross-react with other antigens, and the incubation steps involved mean that it is difficult to detect more than a single antigen in a sample in a short time period.

Another method of detecting the presence of particular target agents in a sample involves detecting the presence of nucleic acids. Several methods of detecting nucleic acids are available including PCR and hybridization techniques. PCR is well known in the art and is described in U.S. Pat. Nos. 4,683,195 and 4,683,202 to Mullis and Mullis et al., respectively. PCR is used for the amplification and detection of low levels of specific nucleic acid sequences. PCR can be used to directly increase the concentration of the target nucleic acid sequence to a more readily detectable level. A variant of PCR is the ligase chain reaction, or LCR, which uses polynucleotides that are ligated together during each cycle. PCR can suffer from non-specific amplification of non-target sequences. Other variants exist, but none have been as widely accepted as PCR.

Hybridization techniques involve detecting the hybridization of two or more nucleic acid molecules. Such detection can be achieved in a variety of ways, including labeling the nucleic acid molecules and observing the signal generated from such a label. Traditional methods of hybridization, including Northern and Southern blotting, were developed with the use of radioactive labels which are not amenable to automation. Radioactive labels have been largely replaced by fluorescent labels in most hybridization techniques. Representative forms of other hybridization techniques include the cycling probe reaction, branched DNA, Invader™ Assay, and Hybrid Capture. However, while overcoming the problem of non-specific nucleic acid amplification associated with PCR, these techniques lack the sensitivity required for many applications, especially infectious disease diagnostics. Also, due to the use of linear amplification, many hybridization techniques can take substantial periods of time to accumulate a detectable signal.

Hybridization techniques may also be used to identify a specific sequence of nucleic acid present in a sample by using microarrays (or “bioarrays”) of known nucleic acid sequences to probe a sample. Such techniques are described in U.S. Pat. No. 6,054,270. Bioarray technologies generally involve attaching short lengths of single stranded nucleic acid to a surface, each unique short chain attached in a specific known location and then adding the sample nucleic acid and allowing sequences present in the sample to hybridize to the immobilized strands. Detection of this hybridization is then carried out by labeling, typically end labeling, of the fragments of the sample to be detected prior to the hybridization. When a sample fragment hybridizes to a specific strand on the array, a signal can be detected from the label, because the position of the hybridization reaction can be detected, and the sequence of the attached strand at that location is known, the sequence of the complementary strand from the sample that has hybridized can be deduced.

Usually the detection of hybridization is by measuring a fluorescent signal; however, methods of detection using an electrochemical detection method have been disclosed. Electrochemical detection methods, and devices used in electrochemical detection methods, are discussed in U.S. Pat. Nos. 5,776,672, 5,972,692, 6,489,160, 6,667,155, 6,670,131, 6,783,935, and 6,818,109, Nakamura, et al., Drug Metab. Pharmaco., 20:3:219-225 (2005); Hashimoto and Ishimori, Lab on a Chip, 1:61-63 (2001); Hashimoto, et al., Anal. Chem., 66:21: 3830-33 (1994); Takahashi, et al., Analyst, 130:687-93 (2005); and Santos-Alvarez, et al., Anal Bioanal. Chem., 378:104-118 (2004) herein incorporated by reference. These electrochemical detection techniques may provide a result in a reduced time period compared to the fluorescent methods of hybridization detection. As discussed above; however, whether fluorescent or electrochemical, hybridization detection methods can be subject to false positives due to non-specific hybridization. Additionally, nucleic acid detection techniques requiring steps of nucleic acid extraction, isolation and purification may lengthen the time taken to achieve a result and also decrease the detection level of the test through the loss of nucleic acid molecules in the many washing steps involved in these isolation steps.

Nucleic acid detection techniques, while overcoming the potential problem of multiplexing associated with ELISA (i.e., the limited number of discriminatory signals), are restricted in use to only detecting nucleic acid. Therefore, agents such as proteins, drugs, hormones, chemical toxins, and prions, which do not contain nucleic acids, cannot be detected by these nucleic acid hybridization techniques. An ideal multiplex detection assay would combine the versatility of antibody recognition with the multiplexing capability and speed of controlled electrochemical detection of nucleic acid hybridization.

Bioterrorism is the intentional use of bacteria, viruses, or toxins to cause disease in human, animal, or plant populations. Advances in biotechnology have made the weaponization and dissemination of bioterrorism agents logistically and financially easier to accomplish. Furthermore, the prevalence of global travel and trade means that a bioterrorism event in one area of the world could quickly spread throughout the world, and pinpointing the epicenter of such an attack could prove difficult without early, rapid, and accurate detection of the bioterrorism agent. A number of nation states have developed their own in-house bioweapons programs, despite international efforts to decrease these programs. As a consequence, bioterrorism agents could be accidentally dispersed, agents could be stolen due to substandard security at many facilities, or information on their creation could be sold by workers. Densely populated urban areas ensure that dissemination of a bioterrorism agent would likely expose a large number of people to the agent. Furthermore, the overuse, or misuse, of many antibiotics has reduced the efficacy of many anti-bacterial agents. In other cases, such as smallpox, the perceived eradication of the disease has led to a reduction in the number of individuals vaccinated, making the population once again vulnerable to a disease for which there is an effective vaccine.

The early and accurate detection and diagnosis of bioterrorism target agents is particularly important for several reasons. Infection with or exposure to many bioterrorism agents initially results in symptoms such as fatigue, respiratory difficulty and muscle pain that are co-symptomatic with more benign ailments such as the flu or common cold, complicating their early diagnosis. Exposure to many bioterrorism agents is rare, and clinicians are either unfamiliar with nuances in the symptoms, or reluctant to diagnose such an exposure given the rarity of these events and the difficulty in confirming the diagnosis. Furthermore, the psychological effects on the community of diagnosing a bioterrorism infection or exposure (even a misdiagnosis) are likely to be substantial, and therefore clinicians might hesitate in diagnosing exposure to a bioterrorism agent without a positive result from a rapid and accurate test.

Infection with or exposure to some bioterrorism agents proceeds with an asymptomatic incubation period, followed by an aggressive progression of symptoms that can quickly lead to death. Early and accurate diagnosis is especially important in these cases because the correct treatment regiment must begin immediately to avoid fatalities. In the event of a mass exposure, stockpiles of medical supplies must be put in transit quickly to reach victims in time for treatment to be effective. In other cases, rapid and accurate diagnosis is important to understanding whether the diseased individual can transmit the ailment to other individuals. Some bioterrorism agents can be spread from human to human, and others cannot, therefore a containment program to limit further exposure to the bioterrorism agent necessarily depends on quickly and accurately diagnosing the affected individuals.

Clearly, an accurate, speedy multiplex detection assay to diagnose exposure to bioterrorism agents is desirable. The present invention provides methods and compositions for such an assay.

### SUMMARY OF THE INVENTION

There is a long standing and recognized need for methods of detecting biological and/or chemical weapons used in bioterrorism. Because many agents that cause various infections or symptoms may be present in trace amounts (low concentrations in biological or environmental samples), traditional antibody-based techniques may fail to detect them specifically or accurately, if at all. Nucleic acid based methods may be ineffective for a variety of reasons including, inter alia, some bioterrorism weapons are chemicals, not biologicals, and nucleic acid based methods of detection do not apply; moreover, even in the case of biological-based bioterrorism weapons, nucleic acid based methods often require a preliminary amplification step, which can take several hours to perform. Hence, there exists a real and long-recognized need for a methodology that facilitates the early detection of chemical and/or biological-based bioterrorism weapons in a reliable, accurate and, preferably, facile manner. The present invention overcomes the failings and shortcomings of the prior known methods.

The present invention provides for the early, rapid and facile detection and/or characterization of an act of bioterrorism through the detection of bioterrorism target agents in the environment and in biologic fluids including, inter alia, saliva, blood, food, water, air and soil. In certain embodiments, the present invention solves the problem of multiplex detection for multiple bioterrorism target agents, while eliminating the need for different tests for chemically-based agents (such as various qualitative methodologies) and biologically-based agents (such as through traditional ligant binding assays or nucleic acid detection). The need for many varied detection methods currently known in the art is overcome by the present invention which provides novel methods that exploit, in a synergistic manner, the high sensitivity and selectivity of antibody:antigen interaction and nucleic acid hybridization using “nonsense” sequences of universal oligos.

In certain embodiments, the present invention provides for early, rapid, facile and accurate detection and/or characterization of a bioterrorism event through the detection of bioterrorism target agents (toxins, viral or bacterial antigens or host antibodies generated against viral or bacterial antigens as the result of a viral or bacterial infection, chemicals, and the like) in biological fluids or environmental or industrial samples. The present invention combines the versatility of antibody recognition with the speed and sensitivity of electrochemical nucleic acid detection, yet reduces or eliminates the need for nucleic acid isolation/amplification and the problems associated with non-specific nucleic acid hybridization. Nucleic acid sequences used for detection in the present invention are rationally designed to minimize non-specific hybridization, and ensure that sequence-specific hybridization is optimized; also, these nucleic acids have sequences unrelated to the bioterrorism agent(s) being detected.

One aspect of the invention provides methods for using a universal chip in the detection of one or more bioterrorism target agents. This embodiment includes the use of (1) a chip-associated universal oligo, (2) a capture-associated universal oligo that is complementary to the chip-associated universal oligo, where the capture-associated universal oligo is conjugated to one or more capture moieties specific for the bioterrorism target agent(s) to be detected, (3) immobilized binding partners to the one or more capture moieties, and (4) a sample suspected of containing the bioterrorism target agent(s). The method includes mixing the sample suspected of containing the target agent(s) with the capture-associated universal oligo to allow the one or more capture moieties to bind the target agent(s) to form a mixture. The mixture is then contacted with immobilized binding partners to the one or more capture moieties. The unreacted capture moieties can react with the immobilized binding partners, thereby removing unreacted capture-associated universal oligos from solution. The resultant solution is then contacted with the chip-associated universal oligo, where a hybridization event between the chip-associated universal oligo and the capture-associated universal oligo indicates that one or more target agents were present in the sample. The hybridization event may be detected by, e.g., electrochemical, fluorescent, or chemiluminescent detection or the like. Preferably, the hybridization is detected by electrochemical means.

Alternatively, the present invention provides an embodiment where the reacted capture moieties are immobilized. This embodiment includes the use of (1) a chip-associated universal oligo, (2) a capture-associated universal oligo that is complementary to the chip-associated universal oligo, where the capture-associated universal oligo is conjugated to one or more capture moieties specific for the bioterrorism target agent(s) to be detected, (3) immobilized binding partners to the bioterrorism target agent or to the capture moiety/bioterrorism target agent complex, and (4) a sample suspected of containing the target agent(s). The method includes mixing the sample suspected of containing the target agent(s) with the capture-associated universal oligo to allow the one or more capture moieties to bind the target agent(s) to form a mixture. The mixture is then contacted with immobilized binding partners to the target agent or capture moiety/target agent complexes. The reacted target agents or complexes can react with the immobilized binding partners, thereby removing reacted capture-associated universal oligos from solution. The immobilized reacted capture-associated universal oligos are separated from the unreacted capture-associated universal oligos still in solution. The immobilized reacted capture-associated universal oligos are then released from immobilization, and then contacted with the chip-associated universal oligo, where a hybridization event between the chip-associated universal oligo and the capture-associated universal oligo indicates that one or more target agents were present in the sample. The hybridization event may be detected by, e.g., electrochemical, fluorescent, or chemiluminescent detection or the like. Preferably, the hybridization is detected by electrochemical means. As in other embodiments, multiple different capture-associated universal oligos can be employed (so-called multiplexing), thereby allowing for the simultaneous screening and detection of multiple bioterrorism target agents from a single sample.

An alternative aspect of the invention provides methods for using a loaded scaffold and a universal chip in the detection of one or more bioterrorism target agents. Use of such loaded scaffolds has been discussed in detail in the co-pending application “Scaffold-Bound Capture Moieties and Uses Thereof,” filed Oct. 6, 2006, U.S. Ser. No. 60/850,016 and is hereby incorporated by reference in its entirety. In embodiments where a loaded scaffold is used, a capture-associated universal oligo is bound to a scaffold instead directly to a capture moiety such as an antigen or antibody and a universal oligo chip is used to detect the presence of bioterrorism target agents in a sample. This embodiment includes the use of (1) an chip-associated universal oligo, (2) a capture-associated universal oligo that is complementary to the chip-associated universal oligo, where the capture-associated universal oligo is associated with a scaffold which also comprises a capture moiety specific for the bioterrorism target agents to be detected (“loaded scaffold”), (3) immobilized binding partners to the capture moiety, and (4) a sample suspected of containing the bioterrorism target agents. The method includes mixing the sample containing the suspected bioterrorism target agents with the loaded scaffold to allow the capture moiety to bind the bioterrorism target agents to form a mixture. The mixture is then contacted with immobilized binding partners to the capture moiety. The unreacted capture moieties can react with the immobilized binding partners, thereby removing unreacted loaded scaffolds from solution. The solution containing the capture-associated universal oligos associated with the reacted loaded scaffolds is then contacted with the chip-associated universal oligo, where a hybridization event between the chip-associated universal oligo and the capture-associated universal oligo indicates that bioterrorism target agents were present in the sample. The hybridization event may be detected by electrochemical detection, by fluorescence detection, or by other methods known in the art. In other embodiments, the capture-associated universal oligos that are associated with the reacted loaded scaffolds may be subjected to a cleavage reaction and/or a linear or logarithmic amplification step after being separated from unreacted loaded scaffolds but before being contacted with the chip-associated universal oligos.

In another aspect of the invention an alternative method for using loaded scaffolds and a universal chip for detection of bioterrorism target agents is provided. This embodiment includes the use of (1) a chip-associated universal oligo, (2) a capture-associated universal oligo that is complementary to the chip-associated universal oligo, where the capture-associated universal oligo is associated with a scaffold which also comprises a capture moiety specific for the bioterrorism target agents to be detected (“loaded scaffold”), (3) immobilized binding partners to the bioterrorism target agents or bioterrorism target agents/capture moiety complex, and (4) a sample suspected of containing the bioterrorism target agents. The method includes mixing the sample containing the suspected bioterrorism target agents with the loaded scaffold to allow the capture moiety to bind the bioterrorism target agents to form a mixture. The mixture is then contacted with immobilized binding partners to the bioterrorism target agents or bioterrorism target agent/capture moiety complex. In this “reverse capture” scenario, the reacted capture moiety reacts with the immobilized binding partners, thereby removing reacted loaded scaffolds from solution. The solution phase containing the unreacted loaded scaffolds is separated from the immobilized phase, the immobilized phase is washed, and the capture-associated universal oligos associated with the reacted loaded scaffolds are then released into solution and contacted with the chip-associated universal oligo, where a hybridization event between the chip-associated universal oligo and the capture-associated universal oligo indicates that bioterrorism target agents were present in the sample. The hybridization event may be detected by electrochemical detection, fluorescence detection, or by other means of detection known in the art. In some embodiments, the capture-associated universal oligos that are associated with the reacted loaded scaffolds may be subjected to a cleavage reaction and/or a linear or logarithmic amplification step after being separated from unreacted loaded scaffolds but before being contacted with the chip-associated universal oligos.

In another aspect of the invention, a “reverse bead/scaffold capture” method for using a universal chip in electrochemical detection of bioterrorism target agents is provided. This embodiment includes the use of (1) a chip-associated universal oligo, (2) a capture-associated universal oligo that is complementary to the chip-associated universal oligo, where the capture-associated universal oligo is associated with a scaffold which also comprises a capture moiety specific for the bioterrorism target agents to be detected (a “loaded scaffold”), (3) immobilized binding partners to the target agent or target agent/capture moiety complex, and (4) a sample suspected of containing the bioterrorism target agents. The method includes mixing the sample containing the suspected bioterrorism target agents with the immobilized binding partner to allow the immobilized binding partner to bind the bioterrorism target agents to form a mixture. The mixture is then contacted with the loaded scaffold to allow the capture moiety on the loaded scaffold to bind the bioterrorism target agents or bioterrorism target agents/immobilized binding partner complex. The capture moieties can react with the appropriate immobilized binding partner/bioterrorism target agent complex to form reacted loaded scaffolds. The solution containing unreacted loaded scaffolds is removed from the immobilized phase, the immobilized phase is washed, and the capture-associated universal oligos associated with the reacted loaded scaffolds may then undergo optional release from the immobilized phase by, e.g., cleavage, and/or linear or logarithmic amplification. The solution containing the capture-associated universal oligos from the reacted loaded scaffolds is then contacted with the chip-associated universal oligo, where a hybridization event between the chip-associated universal oligo and the capture-associated universal oligo indicates that bioterrorism target agents were present in the sample.

An alternative embodiment of the present invention involves amplification of the capture-associated universal oligos. One aspect of this embodiment includes the use of (1) one or more chip-associated universal oligos, (2) one or more capture-associated universal oligos that has the same sequence as or a sequence substantially similar to the respective chip-associated universal oligo, wherein each capture-associated universal oligo comprises a capture moiety specific for the particular bioterrorism target agent to be detected and, optionally, comprises a selectively activatable promoter (e.g., a T7 promoter) or other moiety to enable amplification (e.g., PCR primer site), (3) immobilized binding partners to the capture moiety, to the bioterrorism target agents, or to the capture moiety/bioterrorism target agent complex, (4) a polymerase capable of interacting with said promoter to polymerize the capture-associated universal oligo to produce polymerization products that are complementary or substantially complementary to the chip-associated universal oligos and the capture-associated universal oligos and (5) a sample suspected of containing the bioterrorism target agent(s). The present invention also includes kits that comprise one or more of the forgoing features/elements (1)-(5).

Another aspect of the present invention provides capture moieties of bioterrorism target agents conjugated to oligos for use in the methods of the present invention.

### DESCRIPTION OF THE FIGURES

So that the manner in which the above recited features, advantages and objects of the present invention are attained and can be understood in detail, a more particular description of the invention, briefly summarized above, may be had by reference to the embodiments that are illustrated in the appended drawings. It is to be noted, however, that the appended drawings illustrate only certain embodiments of this invention and are therefore not to be considered limiting of its scope, for the present invention may admit to other equally effective embodiments.

FIG. 1 provides a representative overview flow diagram showing one embodiment of a method for detecting various bioterrorism target agents in accordance with the present invention.

FIG. 2 provides a flow diagram showing a method for selecting universal oligos and universal oligo sets.

FIG. 3 is a schematic diagram demonstrating the detection of a bioterrorism target agent using immobilized binding partners for isolation of the capture-associated universal oligo. A capture moiety (302) is conjugated to a capture-associated universal oligo (306) via a conjugation structure (304) to a form capture-associated universal oligo/capture moiety complex (300). The capture-associated universal oligos (306) are complementary to chip-associated universal oligos (318). The first step (step A) is exposure of the complex (300) to the sample to bind the bioterrorism target agents (308) present in the sample. Reacted complex (310) is illustrated as having bioterrorism target agent (308) bound to the capture moiety (302) of complex (300). The reacted complexes (310) are then exposed to immobilized binding partners (312) for isolation (step B). The immobilized binding partners (312) have a binding partner (314) that is designed to capture a different portion of the target agent (308) than the capture moiety of the complex (300) to form an immobilized binding partner/complex super complex (316). Following isolation of the super complex, the super complex (316) is introduced to the chip-associated universal oligos (318) (step C). D: The binding of the super complex (316) to the chip-associated universal oligos (318) to form a hybridized pair (324) and will generate a signal in, e.g., an electrochemical detection device (322) (step D).

FIG. 4 is a schematic diagram demonstrating the detection of a bioterrorism target agent using immobilized binding partners for isolation of the capture-associated universal oligo/bioterrorism target agent complex. A capture moiety (402) is conjugated to a capture-associated universal oligo (406) via a conjugation structure (404) to a form complex (400). The capture-associated universal oligo (406) is complementary to the chip-associated universal oligos (418). The first step (step A) is exposure of the complex (400) to the sample to bind the bioterrorism target agents (408) in the sample. The reacted complex (410) is illustrated as having bioterrorism target agent (408) bound to the capture moiety (402) of the complex (400). The reacted complexes (410) are then exposed to immobilized binding partners (412) for isolation (step B). The immobilized binding partners (412) have a binding partner (414) that is designed to capture the complex (410) to form an immobilized binding partner/complex super complex (416). Following isolation of the super complex, the super complex (416) is introduced to the chip-associated universal oligos (418) (step C). D: The binding of the super complex (416) to the chip-associated universal oligos (418) to form a hybridized pair (424) and will generate a signal in, e.g., an electrochemical detection device (422) (step D).

FIG. 5 is a schematic diagram illustrating an embodiment of the use of an engineered polymerase recognition site to create multiple copies of the capture-associated universal oligo for more sensitive detection of bioterrorism target agents. Reacted complex (510) contains a capture-associated universal sequence (506), an engineered polymerase site (526), capture moiety (502) which is conjugated to the engineered polymerase site via a conjugation structure (504), and bioterrorism target agent (508) bound to the capture moiety. The binding of an oligonucleotide (528) complementary to the single-stranded polymerase recognition sequence (526) of the capture-associated universal oligo provides a double-stranded polymerase recognition site (step A). The complex is reacted with appropriate nucleotides and a polymerase to provide a double stranded polymerization product (530) (step B). The reactions are carried out to create multiple copies (532) of the capture-associated universal oligo via linear amplification (step. C).

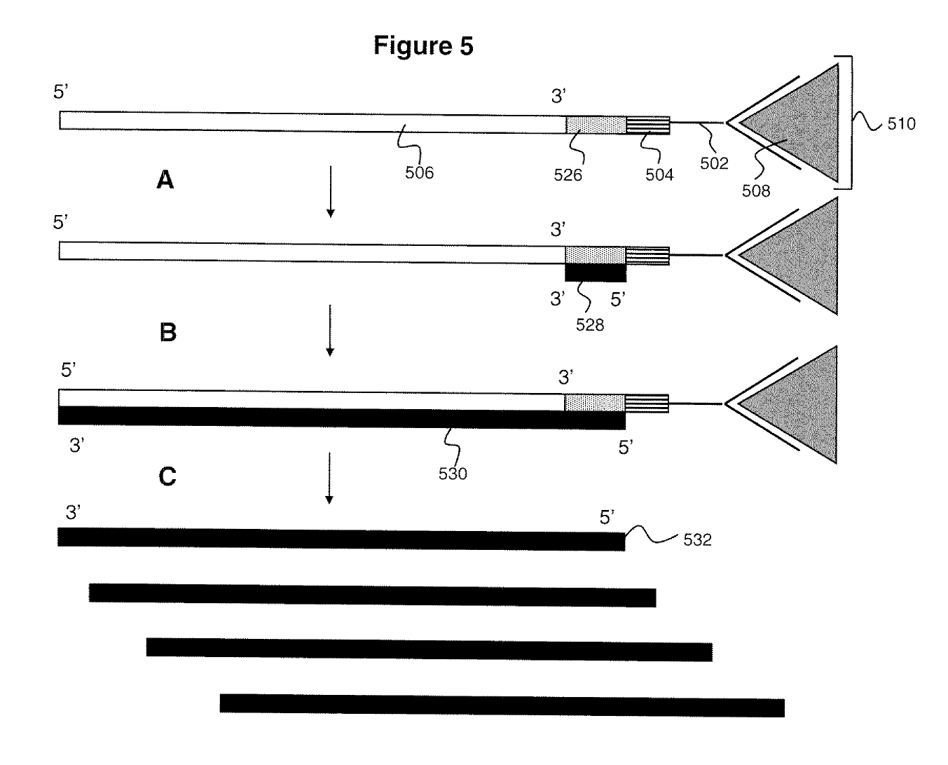

FIG. 6 is a schematic diagram illustrating an embodiment of the use of an engineered capture-associated universal oligo comprising a restriction endonuclease site and a polymerase recognition site. Reacted complex (610) is shown with a capture-associated universal oligo sequence (606), engineered polymerase recognition site. (626), engineered restriction site (634), capture moiety (602) which is bound to the restriction site via a conjugation moiety (604), and bioterrorism target agent (608) bound to the capture moiety. The binding of the oligonucleotide complementary to the encoded single-stranded polymerase recognition sequence (628) and the oligo complementary to the restriction endonuclease cleavage sequence (636) portions of the capture-associated universal oligo provides a double-stranded polymerase recognition site and a restriction endonuclease cleavage site (step A). The complex is reacted with the appropriate restriction endonuclease to remove the capture moiety-bioterrorism target agent complex from the capture-associated universal oligo (step B). The cleaved capture-associated universal oligo is reacted with the appropriate nucleotides and polymerase to provide a polymerization product (630) complementary to the capture-agent associated nucleic acid (step C). The reactions are carried out to create multiple copies (632) of the capture-associated universal oligos via linear amplification (step D).

FIG. 7 is a schematic diagram illustrating an embodiment of the combination of isolation using immobilized binding partners that bind to the bioterrorism target agent and polymerase amplification techniques. Complex (700) contains a capture-associated universal oligo sequence (706), an engineered polymerase site (726), and capture moiety (702) which is conjugated to the engineered polymerase site via a conjugation structure (704). The first step is exposure of the complex to the sample for binding of the bioterrorism target agents (708) in the sample to form reacted complexes (710) (step A). B: Once the capture moiety has bound its bioterrorism target agent, the complex is exposed to immobilized binding partners (712) for isolation to form super complexes (726). Immobilized binding partner (712) has a binding partner (714) that binds to a different portion of the target agent than the capture moiety of the complex. The binding of an oligonucleotide complementary to the encoded single stranded polymerase recognition sequence (728) of the capture-associated universal oligo provides a double-stranded polymerase recognition site (step C). The complex is reacted with the appropriate nucleotides and polymerase to provide a polymerization product (730) complementary to the capture-agent associated universal oligo (step D). The reactions are carried out to create multiple copies (732) of the capture-associated universal oligo via linear amplification (step E). The polymerization products are introduced to the chip-associated universal oligos (not shown). The binding of the polymerization products to the chip-associated universal oligos will generate a signal in, e.g., an electrochemical detection device.

FIG. 8 is a schematic diagram illustrating an embodiment of the combination of isolation using immobilized binding partners that bind to a capture moiety-bioterrorism target agent epitope, restriction endonuclease cleavage of the capture moiety-target agent entities from the capture-associated universal oligo, and polymerase amplification techniques. Complex (800) is shown with a capture-associated universal oligo sequence (806), engineered polymerase recognition site (826), engineered restriction site (834), and capture moiety (802) which is bound to the restriction site via a conjugation moiety (804). The first step is exposure of the complex to the sample suspected of containing the bioterrorism target agent (808) to form a reacted complex (810) (step A). The reacted complexes (810) are then exposed to immobilized binding partners (812) for isolation (step B). Immobilized binding partners (812) have binding partners (814) which bind to the capture moieties of the reacted complexes. The binding of the oligonucleotide complementary to the encoded single-stranded polymerase recognition sequence (828) and the oligo complementary to the restriction endonuclease cleavage sequence portion (842) of the complex provides a double-stranded polymerase recognition site and a restriction endonuclease cleavage site (step C). The complex is reacted with the appropriate restriction endonuclease to cleave the capture moiety-bioterrorism target agent entities (838) from the complex (step D). The cleaved capture-associated universal oligo (840) is reacted with the appropriate nucleotides and polymerase to create a polymerization product (830) complementary to the capture-associated universal oligo (step E). The reactions are carried out to create multiple copies (832) of the capture associated universal oligo via linear amplification (step F). The polymerization products are introduced to the chip-associated universal oligos (not shown). The binding of the polymerization products to the chip-associated universal oligos will generate a signal in, e.g., an electrochemical detection device.

FIG. 9 illustrates one embodiment of the generation of a loaded scaffold. In FIG. 9A, a scaffold (950) is mixed or otherwise contacted with a capture moiety (952) to form a scaffold with an associated capture moiety (954). This scaffold with capture moiety (954) is then mixed or otherwise contacted with capture-associated universal oligos (956) to form a loaded scaffold (958). Loaded scaffold (958) now comprises scaffold (950) with capture moiety (952) and with capture-associated universal oligos (956). In an alternative aspect of this embodiment, capture-associated universal oligos (956) may be added to scaffold (950) first, with capture moieties (954) added subsequently. In FIG. 9B, an alternative embodiment to the method for generating a loaded scaffold (958) is illustrated. Scaffold (950) is mixed or otherwise simultaneously contacted with capture-associated universal oligos (956) and capture moiety (952) to form loaded scaffold (958). The embodiment shown in FIG. 9B differs from that of FIG. 9A in that the capture-associated universal oligo (956) and the capture moiety (952) are simultaneously mixed with scaffold (950) in FIG. 9B versus stepwise in FIG. 9A. In FIG. 9C, an alternative embodiment to the method for generating a loaded scaffold (958) is illustrated. Scaffold (950) is mixed or otherwise contacted with capture-associated universal oligos (956) and capture moiety (952) to form a loaded scaffold (960). The embodiment shown in FIG. 9C differs from that of FIG. 9B in that the loaded scaffold (960) of FIG. 9C is comprised of an increased ratio of capture-associated universal oligo (956) to capture moiety (952) as compared to the loaded scaffold (958) of FIG. 9B. Ratios of capture-associated universal oligos to capture moieties may be varied as needed to optimize detection of various target agents.

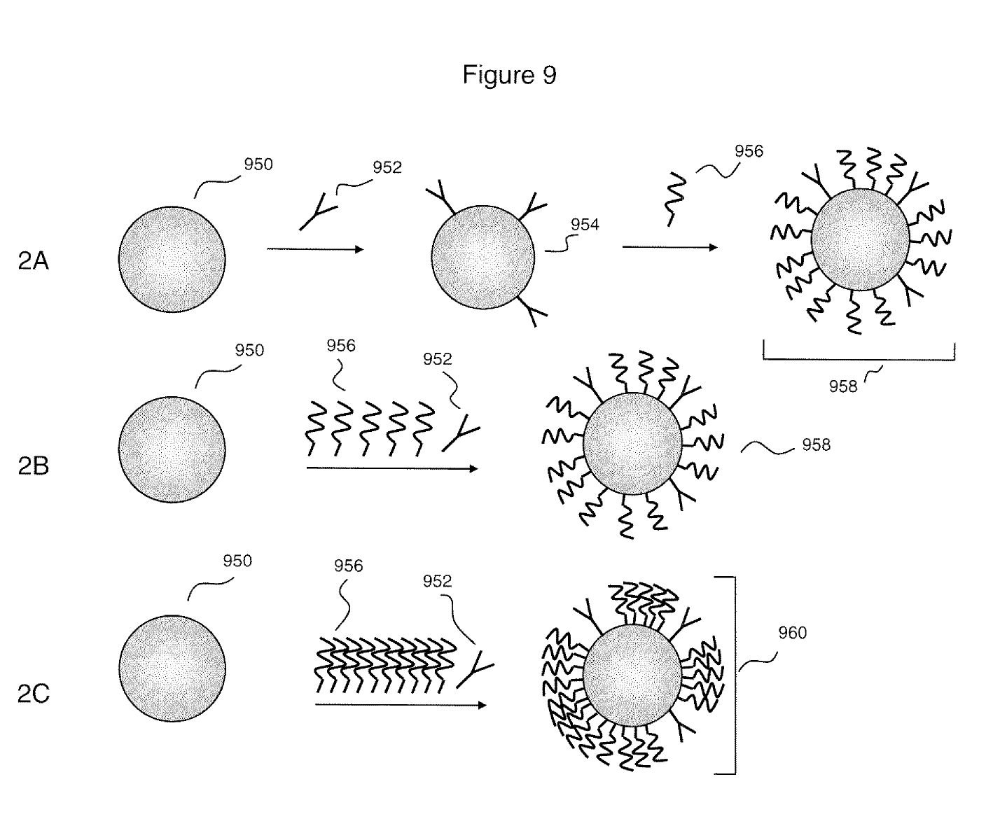

FIG. 10 illustrates one embodiment of using loaded scaffolds for determining the presence of bioterrorism target agent in a sample by electrochemical detection. Capture-associated universal oligos (1056) and capture moieties (1052) are affixed to the surface of the loaded scaffold (1058) (the manufacture of which is described in FIG. 9). In FIG. 10 step A, loaded scaffold (1058) is mixed with or otherwise contacted with a sample suspected of containing bioterrorism target agent (1062) to form reacted loaded scaffold (1064) and unreacted loaded scaffold (1066). The reacted loaded scaffold (1064) comprises loaded scaffold (1058) with at least one target agent (1062) bound to a capture moiety (1052) on the loaded scaffold (1068). The unreacted loaded scaffold (1066) comprises loaded scaffold (1058) with capture moieties (1052) that did not bind to a target agent (1062). In FIG. 10 step B, the products from FIG. 10 step A (reacted loaded scaffold (1064) and unreacted loaded scaffold (1066)) are mixed or otherwise contacted with immobilized binding partners (1068) to form immobilized binding partner/reacted loaded scaffold complexes (1072) and free unreacted loaded scaffolds (1074). The immobilized binding partner (1068) has binding partners (1070) affixed or otherwise attached to the surface of the immobilized binding partners (1068). In this embodiment, the immobilized binding partner (1068) further comprises a magnetic core. The binding partners (1070) of the immobilized binding partners (1068) in this embodiment are designed to bind to a different portion of the target agent (1062) than the capture moiety (1052) of the loaded scaffolds (1058) to form immobilized binding partner/reacted loaded scaffold complexes (1072). The free unreacted loaded scaffolds (1074) comprise unreacted loaded scaffolds (1066) which did not form an immobilized binding partner/reacted loaded scaffold complex (1072) due to the fact that unreacted loaded scaffolds (1066) did not bind a bioterrorism target agent (1062) that is recognized by the immobilized binding partner (1068). In FIG. 10 step C, a magnetic field (1076) is applied across the products of FIG. 10 step B (immobilized binding partner/reacted loaded scaffold complexes (1072) and free unreacted loaded scaffolds (1074)). The magnetic core of the immobilized binding partner (1068) of the immobilized binding partner/reacted loaded scaffold complex (1072) is drawn to the magnetic field. The free unreacted loaded scaffold (1074) is not bound to an immobilized binding partner (1068) and therefore remains in solution. In practice, the reaction represented by FIG. 10 step C may be performed in a reaction container such as a test tube (not shown). Application of the magnetic field (1076) on a side of test tube will draw the magnetized immobilized binding partner/reacted loaded scaffold complexes (1072) to the side of the test tube wall most proximate to the magnetic field (1076), and leave the unmagnetized free unreacted loaded scaffolds (1074) in solution where they may be separated by methods such as aspiration. In FIG. 10 step D, capture-associated universal oligos (1056) from the immobilized binding partner/reacted loaded scaffold complexes (1072) that were magnetically separated from the free unreacted loaded scaffolds (1074) in FIG. 10 step C are released from the loaded scaffolds (1058) and applied to an electrochemical detection device (1082). The electrochemical detection device (1082) comprises one or more electrodes on which chip-associated universal oligos (1080) have been applied. Chip-associated universal oligos (1080) are complementary to the capture-associated universal oligos (1056). Hybridization of chip-associated universal oligos (1080) with capture-associated universal oligos (1056) results in a double stranded nucleotide species (1084) which is subsequently detected.

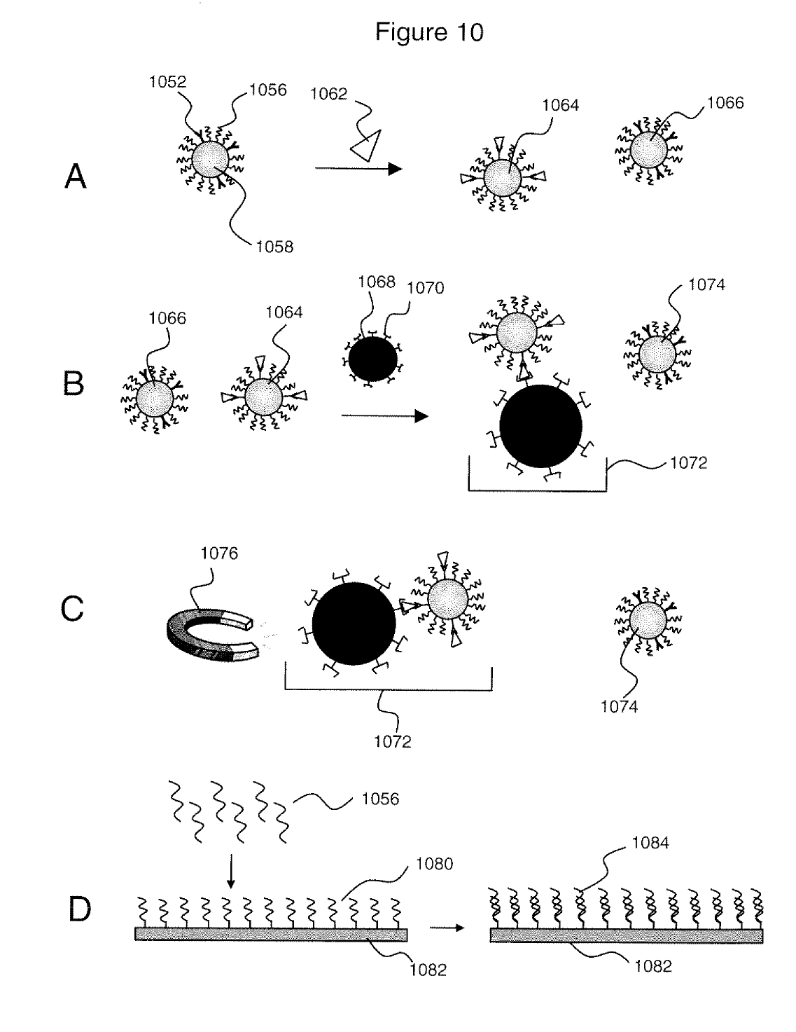

FIG. 11 illustrates a reverse bead capture method where an immobilized binding partner is contacted with a bioterrorism target agent to form a first mixture, then this mixture is contacted with a loaded scaffold. In FIG. 1 step A, binding partner (1170) is immobilized on a magnetic bead to form an immobilized binding partner (1168) that is then is mixed with or otherwise contacted with a sample suspected of containing target agent (1162) to form reacted immobilized binding partner (1186). Reacted immobilized binding partner (1186) is comprised of immobilized binding partner (1168) with bioterrorism target agent (1162) bound to a binding partner (1170). In FIG. 11 step B, the product from the reaction in FIG. 11 step A, the reacted immobilized binding partner (1186), is mixed or otherwise contacted with loaded scaffold (1158) to form a reacted immobilized binding partner/loaded scaffold complex (1188) and an unbound loaded scaffold (1190). Capture-associated universal oligos (1156) are affixed to the surface of the loaded scaffold (1158) (the manufacture of which is described in FIG. 9). The reacted immobilized binding partner/loaded scaffold complex (1188) comprises a loaded scaffold (1158) which has bound to a different portion of the bioterrorism target agent (1162) than the binding partner (1170) of the reacted immobilized binding partner (1186), to form reacted immobilized binding partner/loaded scaffold complex (1188). The unbound loaded scaffold (1190) represents those loaded scaffolds (1158) which did not bind to the bioterrorism target agent (1162) on the reacted immobilized binding partner (1186) due to, e.g., the loaded scaffold being in excess of target agent, or because the capture moiety present on the loaded scaffold did not recognize and bind a target agent present in the sample. In FIG. 11 step C, a magnetic field (1176) is applied across the products of FIG. 11 step B (reacted immobilized binding partner/loaded scaffold complex (1188) and free unbound loaded scaffold (1190)). The magnetic core of the immobilized binding partner (1168) of the reacted immobilized binding partner/loaded scaffold complex (1188) is drawn to the magnetic field. The free unbound loaded scaffold (1190) is not bound to an immobilized binding partner (1168) and therefore remains in solution. In practice, the reaction represented by FIG. 11 step C may be performed in a reaction container such as a test tube (not shown). Application of the magnetic field (1176) on a side of test tube will draw the magnetized reacted immobilized binding partner/loaded scaffold complexes (1188) to the side of the test tube wall most proximate to the magnetic field (1176), and leave the unmagnetized free unbound loaded scaffold (1190) in solution where it may be separated by methods such as aspiration. In FIG. 11 step D, capture-associated universal oligos (1156) from the reacted immobilized binding partner/loaded scaffold complexes (1188) that were magnetically separated from the free unbound loaded scaffold (1190) in FIG. 11 step C are applied to an electrochemical detection device (1182). The electrochemical detection device (1182) comprises one or more electrodes on which electrode-associated universal oligos (1180) have been applied. Electrode-associated universal oligos (1180) are complementary to the capture-associated universal oligos (1156). Hybridization of electrode-associated universal oligos (1180) with capture-associated universal oligos (1156) results in a double stranded nucleotide species (1184) which is subsequently detected.

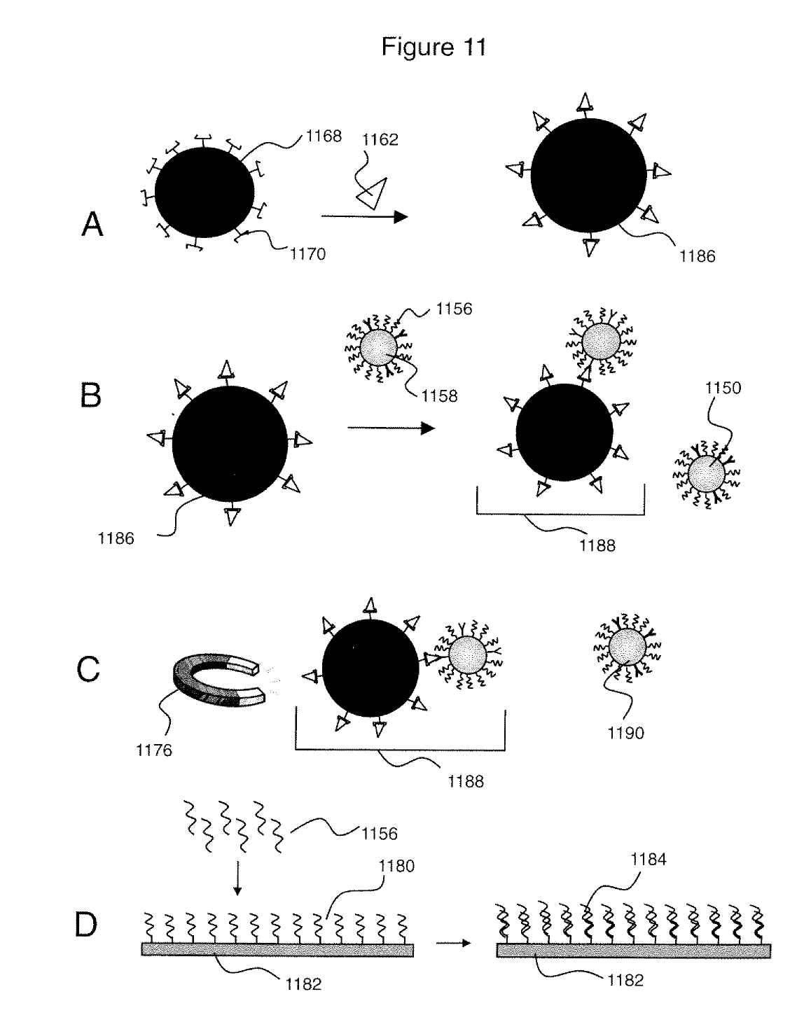

### DEFINITIONS

The terms used herein are intended to have the plain and ordinary meaning as understood by those of ordinary skill in the art unless otherwise specifically defined. The following definitions are intended to aid the reader in understanding the present invention, but are not intended to vary or otherwise limit the meaning of such terms unless specifically indicated.

The term “nucleic acid molecules”, “oligos”, “oligonucleotides” or “polynucleotides” as used herein refers to linear oligomers of natural or modified nucleic acid monomers or linkages, including, inter alia, deoxyribonucleotides, ribonucleotides, anomeric forms thereof, peptide nucleic acid monomers (PNAs), locked nucleotide acid monomers (LNA), and the like, each of which may be capable of specifically binding to a single stranded polynucleotide by way of a regular pattern of monomer-to-monomer interactions, such as Watson-Crick type of base pairing, base stacking, Hoogsteen or reverse Hoogsteen types of base pairing, or the like. Typically monomers are linked by phosphodiester bonds or analogs thereof to form oligonucleotides ranging in size from a few monomeric units, e.g., 8-12, larger numbers of monomeric units, e.g., 100-200 ad even larger, e.g., 100-9000. Suitable nucleic acid molecules may be prepared by the phosphoramidite method described originally by Beaucage and Carruthers (Tetrahedron Lett., 22, 1859-1862, 1981), or by the triester method described originally by Matteucci, et al. (J. Am. Chem. Soc., 103, 3185, 1981), both incorporated herein by reference, or by other chemical methods such as using a commercial automated oligonucleotide synthesizer and/or other known methodologies. “Capture-associated universal oligos” refers to oligos that are conjugated to or otherwise associated with a capture moiety. “Chip-associated universal oligos” refers to oligos that are immobilized on or otherwise associated with a substrate but need not be limited to immobilization on a “chip.” In certain embodiments, the chip-associated universal oligo is immobilized on surfaces other than chips including, inter alia, reaction vessels, filters, membranes, beads, and the like. In other embodiments, the “chip-associated” oligo is not immobilized but is captured together with its complementary strand, for example by use of an antibody that specifically recognizes a RNA:DNA hybrid duplex (discussed further, infra). In certain preferred embodiments the chip-associated universal oligo is immobilized on or otherwise associated with a chip.

The terms “complementary” or “complementarity” are used in reference to nucleic acid molecules (i.e., a sequence of nucleotides) that are related by base-pairing rules. Complementary nucleotides are, generally, A and T (or A and U), or C and G. Two single stranded RNA or DNA molecules are said to be substantially complementary when the nucleotides of one strand, optimally aligned and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Selective hybridization conditions include, but are not limited to, stringent hybridization conditions. Selective hybridization typically refers to embodiments wherein there is at least about 65% complementarity over a stretch of at least 14 to 25 nucleotides, preferably at least about 75%, more preferably at least about 90% complementarity. See, M. Kanehisa, Nucleic Acids Res. 12, 203 (1984), incorporated herein by reference. For shorter nucleotide sequences selective hybridization occurs when there is at least about 65% complementarity over a stretch of at least 8 to 12 nucleotides, preferably at least about 75%, more preferably at least about 90% complementarity. Stringent hybridization conditions will typically include salt concentrations of less than about 1 M, more preferably less than about 500 mM and preferably less than about 200 mM. Hybridization temperatures can be as low as −80° C., preferably greater than about 5° C., and are preferably lower than about 30° C. However, longer fragments may require elevated hybridization temperatures for specific hybridization. Hybridization temperatures are generally at least about 1° C. to 20° C. lower than melting temperatures (Tₘ), which is defined below, preferably about 1° C. to about 12° C. lower than melting temperature, and more preferably about 2° C. to about 8° C. lower than melting temperature.

The term “universal oligo” generally refers to one oligonucleotide of a complementary oligonucleotide pair, where each oligonucleotide in the pair has been rationally designed to have low complementarity to all sequences that may be present in a sample. For example, in a blood sample for diagnosis of bioterrorism in a human, a universal oligo would be one with low complementarity to human genomic sequences, genomic sequences from biological organisms associated with bioterrorism agents, as well as genomic sequences of organisms that associate with humans (e.g., human gut flora). For a soil sample, a universal oligo would be one with minimal complementarity to genomic sequences from, e.g., soil flora and fauna. A “universal oligo set” is a set of two or more universal oligo pairs where each oligo in the set has low complementarity to every other universal oligo in the set, with the exception of its complement. A “universal oligo chip” is an array of two or more universal oligos—each from a different universal oligo pair—that are immobilized at a known location on a surface such as glass, plastic, nylon, silicon, etc. The term “capture-associated universal oligo” refers to the oligo of a universal oligo pair that is associated with a capture moiety or a scaffold. The term “chip-associated universal oligo” refers to the oligo of a universal oligo pair that is immobilized on or otherwise associated with a substrate but need not be limited to immobilization on a “chip.” In certain embodiments, the chip-associated universal oligo is immobilized on surfaces other than chips including, inter alia, reaction vessels, filters, membranes, beads, and the like. In other embodiments, the “chip-associated” oligo is not immobilized but is captured together with its complementary strand, for example by use of an antibody that specifically recognizes a RNA:DNA hybrid duplex (discussed further, infra). In certain preferred embodiments the chip-associated universal oligo is immobilized on or otherwise associated with a chip.

In certain embodiments of the present invention, capture-associated universal oligos and chip-associated universal oligos are complementary or substantially complementary; however, in embodiments where linear amplification of the capture-associated universal oligo is employed (as described in detail infra), the capture-associated universal oligos and the chip-associated universal oligos are complementary or substantially complementary and the amplification products derived from the capture-associated universal oligo are complementary to the capture-associated universal oligos and the chip-associated universal oligos.

The term “capture” is intended to convey any association, including, inter alia, conjugation, irreversible binding, reversible binding, covalent binding, intercalation, non-covalent binding, etc. A “capture moiety” refers to a portion of a molecule that can be used to preferentially associate with or bind to and separate a molecule of interest (a “target agent”) present in or potentially present in a sample. The term “capture moiety” as used herein refers to any molecule, natural, synthetic, or recombinantly produced, with the ability to bind to the target agent in any of the methods of the present invention. The binding affinity of the capture moiety must be sufficient to allow collection of the target agent from a sample. Suitable capture moieties include, inter alia, antibodies, antigen-binding regions of antibodies, antigens, epitopes, cell receptor ligands, such as peptide growth factors (see, e.g., Pigott and Power (1993), The Adhesion Molecule Facts Book (Academic Press New York); and Receptor Ligand Interactions: A Practical Approach, Rickwood and Hames (series editors) Hulme (ed.) (IRL Press at Oxford Press NY)). Similarly capture moieties may also include but are not limited to toxins, venoms, intracellular receptors (e.g., receptors that mediate the effects of various small ligands, including steroids, hormones, retinoids and vitamin D, peptides), drugs (e.g., opiates, steroids, etc.), lectins, sugars, oligosaccharides, other proteins, and phospholipids. Persons of ordinary skill in the art readily will appreciate that other and varied capture moieties based upon other molecular interactions than those listed above are well described in the literature and may also serve as capture moieties.

By “preferentially binds” it is meant that a capture moeity is designed to be at least 5-20 times or more, preferably 20-50 times or more, more preferably 50-100 times or more, and even more preferably 100-1000 times or more likely to bind to the intended target agent than to other molecules in a biological solution. In the embodiment where the capture moiety is comprised of antibody, the binding affinity may be due to (1) a single monoclonal antibody (i.e., large numbers of one kind of antibody) or (2) a plurality of different monoclonal antibodies (e.g., large numbers of each of five different monoclonal antibodies) or (3) large numbers of polyclonal antibodies. It is also possible to use combinations of (1)-(3). The four-fold differential in binding affinity may be accomplished by using several different antibodies as per (1)-(3) above and as such some of the antibodies in a mixture could have less than a four fold difference. For purposes of the invention an indication that no binding occurs means that the equilibrium or affinity constant Kₐ is 10⁶ l/mole or less. Antibodies may be designed to maximize binding to the intended antigen by designing the peptides to specific epitopes that are more accessible to binding, as can be predicted by one skilled in the art.

A “bioterrorism target agent” or “target agent” is intended to refer to any target moiety in a sample that is to be captured through preferential binding with a capture moiety. For example, in the case where the capture moiety is an antibody, the bioterrorism target agent will be any molecule which contains the epitope against which the antibody is generated. Where the capture moiety is a protein used for detection of an antibody, the antibody itself is the target agent. Thus, the bioterrorism target agent may be the chemical or biological weapon itself, or the bioterrorism target agent may be an antibody generated by an exposed individual, a metabolite generated by an exposed individual, a by-product of the chemical or biological weapon, and the like. In some embodiments, the target agent may be an environmental pollutant (including pesticides, insecticides, toxins, etc.); a chemical (including solvents, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including prokaryotic (such as pathogenic bacteria) and eukaryotic cells, including mammalian tumor cells); viruses (including retroviruses, herpesviruses, adenoviruses, lentiviruses, etc.); and spores; etc. In the present invention, bioterrorism target agents are detected. Bioterrorism markers (target agents) include virtually any toxin and biological molecule such as antibodies, antigens, cytokines, proteins, lipids, carbohydrates, cellular membrane antigens and receptors, metabolites, nucleic acids, enzymes and the like. See, e.g., Lim et al., Clinical Microbiology Review, 18:583 (2005). A representative non-limiting listing of suitable bioterrorism target agents includes, inter alia:

TABLE 1a

| | Representative Target Agents (in addition to |
|---|---|
| | the causative organism itself, its nucleic acid |
| Organism | sequences, and other defining characteristics) |
| Bacillus anthracis | Anti-PA (protective antigen); anti-PA IgG; |
| (anthrax) | anti-LF (lethal factor) IgG; protective antigen; |
| | lethal factor; edema factor; capsule peptide (γ- |
| | D-glutamic acid (γDPGA)); anti- γDPGA IgG; |
| | cell wall antigen; capsule antigen; spore |
| | antigen. |
| Clostridium botulinum | Anti-Clostridium botulinum Toxin A; |
| (botulism toxin) | Clostridium botulinum B Toxoid; Clostridium |
| | botulinum C Toxoid; Anti-Clostridium |
| | botulinum D Toxoid; Clostridium botulinum E |
| | Toxoid; Clostridium botulinum F Toxoid. |
| Brucella species | Antibody for Brucella abortus smooth |
| (brucellosis) | lipopolysaccharide (S-LPS), S-LPS antigen; |
| | non-S-LPS antigen; IgG, IgA antibodies. |
| Burkholderia mallei | Cell-associated antigens include |
| (glanders) | exopolysaccharide (EPS) lipopolysaccharide |
| | (LPS); LPS type I O-PS and LPS type II O-PS; |
| | LPS O-antigen antibody; IgG. |
| Burkholderia | Cell-associated antigens include |
| pseudomallei | exopolysaccharide (EPS) lipopolysaccharide |
| (melioidosis) | (LPS); LPS type I O-PS and LPS type II O-PS; |
| | LPS O-antigen antibody; IgG, IgM. |
| Chlamydophila psittaci | IgM, IgG antibody; lipopolysaccharide antigen |
| (psittacosis) | |
| Vibrio cholerae | Cholera toxin alpha subunit; cholera toxin beta |
| (Cholera) | subunit; LPS epitopes of Vibrio cholerae |
| | antigens present in the bacterial cell wall; |
| | capsular polysaccharide; IgG. |
| Clostridium perfringens | Toxin beta; enterotoxin A; alpha toxin; epsilon |
| (Epsilon toxin) | toxin; IgG, IgM. |
| Coxiella burnetii | Smooth lipopolysaccharide; rough |
| (Q fever) | lipopolysaccharide; anti-Q fever antibody; IgG, |
| | IgA, IgM. |
| Ebola virus | VP40; IgG, IGM antibodies; Ebola |
| | nucleoprotein (NP); viral glycoprotein GP. |
| Escherichia coli | O157 antigen; H7 antigen; IgG, IgM. |
| O157:H7 | |
| Nipah virus | PCR, antibody, antigen |
| Hantavirus | Nucleocapsid protein; glycoprotein G2; IgG, |
| | IgM. |
| Salmonella species | Somatic (O) antigen; flagellar (H) antigen; |
| (salmonellosis) | lipopolysaccharides A, B, C, D, E; Salmonella |
| | enteriditis D 0-9 antigen; outer membrane |
| | polysaccharide (K) antigen; A, B, & D group |
| | specific antigen (O-12) of Salmonellae LPS; |
| | A-group 0-2 antigen; B-group 0-4 antigen; core |
| | antigen; IgG, IgA, IgM. |
| Shigella (shigellosis) | IgA and IgG anti-Shigella lipopolysaccharide; |
| | somatic (O) antigen; |
| Francisella tularensis | Lipopolysaccharide; lipopolysaccharide |
| (tularemia) | protein; O antigen polysaccharide chain; IgG, |
| | IgM, IgA. |
| Lassa fever | IgM, IgG; zinc binding (Z) protein; |
| | nucleoprotein; |
| Marburg virus | VP40; IgG, IgM antibodies |
| Yersinia pestis | F1 capsular antigen; V antigen; IgG, IgM, IgA. |
| (plague) | |
| Ricinus communis | A-chain; agglutinin 60; RCA120; IgG, IgM. |
| (ricin toxin) | |
| Rickettsia prowazekii | lipopolysaccharide (LPS); 1-2 outer-membrane |
| (typhus fever) | protein (rOmpA and/or rOmpB); |
| | lipopolysaccharide-like (LPS-L) antigen; IgG, |
| | IgM, IgA. |
| Smallpox | A27L protein; B5R protein; IgG, IgM, IgA. |
| Staphylococcal | Enterotoxin B protein; IgG, IgM, IgA. |
| enterotoxin B | |
| Machupo virus | GP-1 and GP-2 structural proteins; |
| | nucleoprotein; IgG, IgM, IgA. |
| Cryptosporidium parvum | Surface glycoprotein; substrate adhesion |
| | molecule; inner oocyst wall antigen; IgG, IgM, |
| | IgA. |

TABLE 1b

| Chemical | Representative Target Agents |
|---|---|
| Botulinum toxin | Botulinum toxin, metabolites thereof, |
| | degradation products thereof. |
| Abrin | Abrin, metabolites thereof, degradation |
| | products thereof. |
| Ricin | Ricin, metabolites thereof, degradation |
| | products thereof. |
| Saxitoxin | Saxitoxin, metabolites thereof, degradation |
| | products thereof. |
| Arsines | Arsines, metabolites thereof, degradation |
| | products thereof. |
| Cyanogen chloride | Cyanogen chloride, metabolites thereof, |
| | degradation products thereof. |
| Hydrogen cyanide | Hydrogen cyanide, metabolites thereof, |
| | degradation products thereof. |
| Lewisite | Lewisite, metabolites thereof, degradation |
| | products thereof. |
| Phosgene oxime | Phosgene oxime, metabolites thereof, |
| | degradation products thereof. |
| Sulfur mustard gas | Sulfur mustard gas, metabolites thereof, |
| | degradation products thereof. |
| Nitrogen mustard gas | Nitrogen mustard gas, metabolites thereof, |
| | degradation products thereof. |
| Tabun | Tabun, metabolites thereof, degradation |
| | products thereof. |
| Sarin | Sarin, metabolites thereof, degradation |
| | products thereof. |
| Soman | Soman, metabolites thereof, degradation |
| | products thereof. |
| Cyclosarin | Cyclosarin, metabolites thereof, degradation |
| | products thereof. |
| Chloropicrin | Chloropicrin, metabolites thereof, degradation |
| | products thereof. |
| Chlorine | Chlorine, metabolites thereof, degradation |
| | products thereof. |
| Diphosgene | Diphosgene, metabolites thereof, degradation |
| | products thereof. |
| Dimethyl | Dimethyl methylphosphorate, metabolites |
| methylphosphorate | thereof, degradation products thereof. |
| Agent 15 | Agent 15, metabolites thereof, degradation |
| | products thereof. |
| KOLOKOL-1 | KOLOKOL-1, metabolites thereof, degradation |
| | products thereof. |
| CS gas | CS gas, metabolites thereof, degradation |
| | products thereof. |
| CN gas | CN gas, metabolites thereof, degradation |
| | products thereof. |
| CR gas | CR gas, metabolites thereof, degradation |
| | products thereof. |
| Pepper spray | Pepper spray, metabolites thereof, degradation |
| | products thereof. |
| Novichok gas | Novichok gas, metabolites thereof, degradation |
| | products thereof. |
| Tear gas | Tear gas, metabolites thereof, degradation |
| | products thereof. |
| Dioxins | Dioxins, metabolites thereof, degradation |
| | products thereof. |
| Agent orange | Agent orange, metabolites thereof, degradation |
| | products thereof. |
| Napalm | Napalm, metabolites thereof, degradation |
| | products thereof. |

The term “sample” in the present specification and claims is used in its broadest sense and can be, by non-limiting example, any sample that is suspected of containing the target agents to be detected. It is meant to include a specimen or culture (e.g., microbiological cultures), biological and environmental samples. Biological samples may comprise animal derived materials, including human, fluid, solid (e.g., stool) or tissue, as well as liquid and solid food and feed products and ingredients such as dairy items, vegetables, meat and meat by-products, and waste. Biological samples may be obtained from any domestic or wild animals. Environmental samples can include environmental material such as surface matter, soil, water, air and industrial samples, as well as samples obtained from food and dairy processing instruments, apparatus, equipment, utensils, disposable and non-disposable items. These examples are not to be construed as limiting the sample types applicable to the present invention. Those of skill in the art would appreciate and understand the particular type of sample required for the detection of particular target agents (see, e.g., Tietz Textbood of Clinical Chemistry and Molecular Diagnostics, 4^{th} Ed., Chapter 2, Burtis, C. Ashwood E. and Bruns, D, eds. (2006); Chemical Weapons Convention Chemicals Analysis: Sample Collection, Preparation and Analytical Methods, Mesilaakso, M., ed., (2005); Pawliszyn, J., Sampling and Sample Preparation for Field and Laboratory, (2002); Venkatesh Iyengar, G., et al., Element Analysis of Biological Samples: Priniciples and Practices (1998); Drielak, S., Hot Zone Forensics: Chemical, Biological, and Radiological Evidence Collection (2004); Wells, D., High Throughput Bioanalytical Sample Preparation (Progress in Pharmaceutical and Biomedical Analysis) (2002); and Nielsen, D. M., Practical Handbook of Environmental Site Characterization and Ground-Water Monitoring (2005)).

The term “antibody” as used herein is intended to refer to an entire immunoglobulin or antibody or any functional fragment of an immunoglobulin molecule which is capable of specific binding an antigen. Antibody as used herein is meant to include the entire antibody as well as any antibody fragments capable of binding the antigen or antigenic fragment of interest. Examples of such peptides include complete antibody molecules, antibody fragments, such as Fab, F(ab′)₂, CDRS, V_{L}, V_{H}, and any other portion of an antibody which is capable of specifically binding to an antigen. Preferred antibodies for assays of the invention are immunoreactive or immunospecific for, and therefore specifically and selectively bind to, a bioterrorism target agent. A “purified antibody” refers to that which is sufficiently free of other proteins, carbohydrates, and lipids.

A substance is commonly said to be present in “excess” or “molar excess” relative to another component if that component is present at a higher molar concentration than the other component. Often, when present in excess, the component will be present in at least a 10-fold molar excess and commonly at 100-1,000,000 fold or greater molar excess. Those of skill in the art would appreciate and understand the particular degree or amount of excess preferred for any particular reaction or reaction conditions. Such excess is often empirically determined and/or optimized for a particular reaction or reaction conditions. The specific degree of excess preferred for any particular diagnostic will be readily understood by those of ordinary skill in the art.

The term “reacted nucleic acid molecules” or “reacted molecules” is used in reference to those nucleic acid molecules that have a conjugated capture moiety for a particular target agent, where the target agent is present in the sample, and the corresponding capture moiety has bound to the target agent. The term “unreacted nucleic acid molecules” or “unreacted molecules” is used in reference to those nucleic acid molecules that have a conjugated capture moiety for a particular target agent, but the target agent was not present in the sample—or was present in an amount less than the capture moiety—and the corresponding capture moiety has not bound the particular target agent.

The term “capture reaction” may be used in reference to the mixing/contacting of the nucleic acid molecules conjugated to a capture moiety and the sample under conditions that allow the capture moiety to attach to, bind or otherwise associate with a target agent in the sample.

The term “melting temperature” or Tm is commonly defined as the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tₘ=81.5+16.6(log₁₀[Na⁺])0.41(%[G+C])−675/n−1.0m, when a nucleic acid is in aqueous solution having cation concentrations of 0.5 M, or less, the (G+C) content is between 30% and 70%, n is the number of bases, and m is the percentage of base pair mismatches (see e.g., Sambrook J et al., “Molecular Cloning, A Laboratory Manual, 3^{rd} Edition, Cold Spring Harbor Laboratory Press (2001)). Other references include more sophisticated computations, which take combinations of structural characteristics as well as sequence characteristics and reaction conditions into account for the calculation of a particular Tm.

The term “matrix” means any surface. Suitable matrices include those made from, inter alia, glass, nylon, polymethylacrylamide, polystyreme, polyvinyl chloride, latex, chemically modified plastic, cellulose, rubber, red blood cells, polymeric materials or biological materials.

A “restriction endonuclease” is any enzyme capable of recognizing a specific sequence (the “restriction site”) on a double- or, preferably, single-stranded polynucleotide and cleaving the polynucleotide at or near the site. Examples of site-specific restriction endonucleases are available in the 2006 New England Biolabs, Inc. Catalog, including the 2006 New Products Catalog Supplement, which is incorporated herein by reference. The term “moiety that is capable of creating a signal” encompasses virtually any of the signal generating systems used in the prior art and any system to be developed in the future. It comprises a moiety which generates a signal itself, e.g., a dye, a radioactive molecule, a chemiluminescent material, a fluorescent material or a phosphorescent material, or a moiety which upon further reaction or manipulation will give rise to a signal, e.g., an enzyme linked system. Suitable enzymes that can be utilized to create a signal are essentially any enzyme that is capable of generating a signal when treated with a suitable reagent. Preferred enzymes are horseradish peroxidase, alkaline phosphatase, glucose oxidase, peroxidase, acid phosphatase and beta-galactosidase. Such enzymes are preferred because they are very stable, yet highly reactive. Another method in which the target genetic material can be detected is a method in which each single stranded polynucleotide segment has a label, and a when a double hybrid is formed, the combination of the labels from each single stranded polynucleotide segment creates a signal; i.e., neither label of each polynucleotide alone is capable of creating a signal. In this system it is preferred that each of the two labels be attached, either covalently or via complex formation, at one end of each single stranded polynucleotide segment where when the hybrid is formed, the labels are proximate one another. Thus, in one embodiment, the first label is attached in the three prime terminal position of one single stranded polynucleotide segment and the second label is attached at the five prime terminal position of the other single stranded polynucleotide segment. In a more preferred embodiment the label of each polynucleotide is capable of forming a complex, thereby increasing the proximity of the two labels and resulting in a stronger signal. Such affinity or complex formation can be naturally occurring, e.g., where an apoenzyme is one label and the apoenzyme's cofactor is the other label. In this system a signal can be created by adding a suitable reagent, but such signal is only created if the apoenzyme and its cofactor form a complex. Alternatively, the affinity or complex can be artificially created. For example, one label can be a chemiluminescent catalyst and the other label can be an absorber/emitter moiety. The oligonucleotides hybridize to each other placing the chemiluminescent catalyst and absorber/emitter moiety in proximity to produce a detectable signal. These methods can be carried out as described, by non-limiting example, in European Patent Application Publication Number 0 070 685, published Jan. 26, 1983, the disclosure of which is incorporated herein. Each of the ligand and receptors disclosed hereinabove can be utilized to create the artificial affinity.

The term “binding partner” refers to a portion of a molecule that preferentially binds to a separate region on the target agent than the capture moiety, such that both the capture moiety and the binding moiety may be simultaneously bound to the target agent. A “binding partner” may also preferentially bind to a capture moiety/target agent complex. Alternatively, in an embodiment where unreacted loaded scaffolds are captured by immobilized binding partners (rather than reacted loaded scaffolds being captured by immobilized binding partners), the immobilized binding partners will bind unreacted capture moieties. The term “binding partner” as used herein refers to any molecule, natural, synthetic, or recombinantly produced, with the ability to bind to the target agent and/or capture moiety in the methods of the present invention. The binding affinity of the binding partner must be sufficient to allow collection of the target agent and/or capture moiety from a sample and/or sample mixture. Suitable binding moieties include, but are not limited to, antibodies, antigen-binding regions of antibodies, antigens, epitopes, cell receptor ligands, such as peptide growth factors (see, e.g., Pigott and Power (1993), The Adhesion Molecule Facts Book (Academic Press New York); and Receptor Ligand Interactions: A Practical Approach, Rickwood and Hames (series editors) Hulme (ed.) (IRL Press at Oxford Press NY)). Similarly, binding partners may also include but are not limited to toxins, venoms, intracellular receptors (e.g., receptors which mediate the effects of various small ligands, including steroids, hormones, retinoids and vitamin D, peptides), drugs (e.g., opiates, steroids, etc.), lectins, sugars, oligosaccharides, other proteins, and phospholipids. Those of skill in the art readily will appreciate that a number of binding partners based upon molecular interactions other than those listed above are well described in the literature and may also serve as binding partner. The binding partners can be affixed/immobilized directly or indirectly to a matrix such as a vessel wall, to particles or beads (as described in more detail infra), or to other suitable surfaces to form “immobilized binding partners”. Those of skill in the art will readily understand the versatility of the nature of this immobilized binding partner. Essentially, any ligand and receptor can be utilized to serve as capture moieties, target agents and binding partners, as long as the target agent is appropriate for detection for the pathology or condition interrogated. Suitable ligands and receptors include an antibody or fragment thereof to be recognized by a corresponding antigen or epitope, a hormone to be recognized by its receptor, an inhibitor to be recognized by its enzyme, a co-factor portion to be recognized by a co-factor enzyme binding site, a binding ligand to be recognized by its substrate, and the like.

The term “scaffold” as used herein describes a support upon which capture-associated universal oligos and capture moieties are bound. Such support can include, but is not limited to, such structures as gold, aluminum, copper, platinum, silica, titanium dioxide, carbon nanotubes, polystyrene particles, polyvinyl particles, acrylate and methacrylate particles, glass particles, latex particles, Sepharose beads and other like particles, polymer coated magnetic beads, semiconducting materials, and radio frequency identification substrates.

The term “loaded scaffold” refers to a scaffold that comprises both capture-associated universal oligos and capture moieties affixed or otherwise associated with the scaffold.

The term “reacted loaded scaffolds” is used in reference to those loaded scaffolds where the capture moiety on the loaded scaffold has bound to a bioterrorism target agent from a sample. The term “unreacted loaded scaffolds” is used in reference to those loaded scaffolds where the capture moiety associated with the loaded scaffold has not bound to a bioterrorism target agent.

The terms “SAM” and “self-assembled monolayer”, as used interchangeably throughout the specification, refers to crystalline chemisorbed organic single layers formed on a solid substrate by spontaneous organization of the molecules.

An “epitope” of a molecule means a portion of such a molecule which is capable of preferentially binding to a capture moiety.

The term “electrode” as used herein means a composition which, when connected to an electronic device, is able to conduct, transmit, receive or otherwise sense a current or charge. This current or charge is subsequently converted into a detectable signal. Alternatively an electrode can be defined as a composition which can apply a potential to and/or pass electrons to or from a chemical moiety.

A “biosensor” is defined as being a substrate comprising (1) one or more moieties for necessary for molecular recognition, e.g., a chip-associated oligo that preferentially binds to a capture-associated oligo (2) a surface onto which the moieties for molecular recognition are associated; and (3) a transducer for transmitting the recognition information to processable signals. A preferred biosensor for use in the methods of the invention is an electrochemical detection device, which comprises an electrode and an electrode-associated oligo.

The term “chip” as used herein refers to an object for detection of the binding of a universal oligo pair, where the chip comprises a surface and one or more universal oligos associated to this surface.

An “anchoring group” as defined herein refers to a component of a SAM that is associated with a moiety for molecular recognition. The anchoring group serves to attach the moiety for molecular recognition (e.g., an oligo) to the signal transducer (e.g., an electrode).

A “diluent group” as defined herein refers to any component of a SAM that is not associated with a moiety for molecular recognition.

A “detection moiety” is any one or a plurality of chemical moieties capable of enabling the molecular recognition on a biosensor. In certain embodiments, the detection moiety can be any chemical moiety that is stable under assay conditions and can undergo reduction and/or oxidation.

It should be understood by those skilled in the art that terms such as “target”, “agent”, “moiety”, “antigen”, “antibody”, “molecule” and the like should be interpreted in the context in which they appear, and should be given the broadest interpretation possible unless specifically indicated.

### DETAILED DESCRIPTION OF THE INVENTION

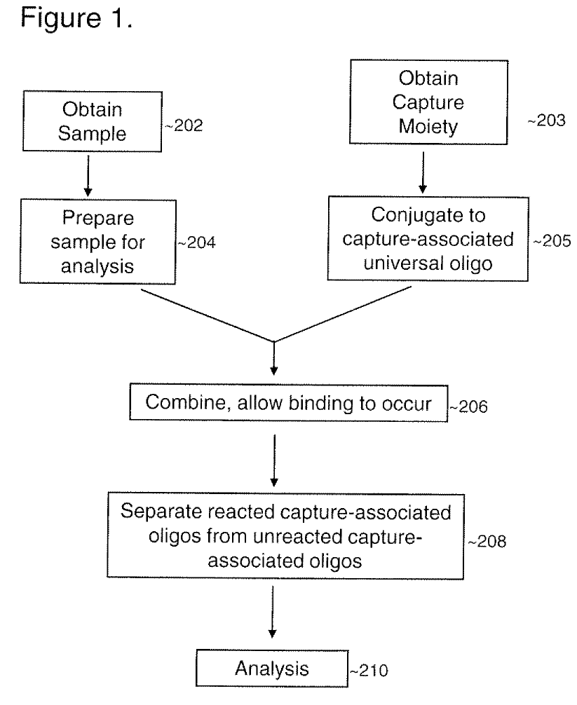

The present invention relates to compounds and methods of use for early detection or characterization of acts of bioterrorism by detecting and/or diagnosing exposure to or infection by chemical or biological weapons. One embodiment of the present invention is outlined in representative overview in FIG. 1. FIG. 1 illustrates a method 200, comprising the steps of obtaining a sample suspected of containing bioterrorism target agents, in this case, antigenic compounds such as proteins, other chemicals, metabolites and the like (202). The sample may be blood, for example. The sample is prepared for analysis (204). One or more antibodies to the target agent are obtained (203), and are conjugated to the one or more antibodies to capture-associated universal oligos (205). The prepared sample and the conjugated universal oligos are then combined allowing binding to occur (206), the reacted capture-associated universal oligos from unreacted universal oligos are separated (208), and the reacted universal oligos, if any, are analyzed (210).

Alternatively, both the capture moieties and the capture-associated universal oligos are affixed or otherwise associated with a scaffold, and universal oligo chips may be used in a system comprising loaded scaffolds, where the capture moiety on the loaded scaffold is, for example, an antibody, antigen or other ligand specific for a particular bioterrorism target agent. Briefly, the loaded scaffold is contacted/mixed with a sample that is suspected of containing the target agents, under conditions that if a target agent is present, the capture moiety can react with, i.e., bind with/to the specific target agent. The capture-associated universal oligos associated with the scaffold are added in excess relative to the amount of target agent suspected to be present in the sample in many embodiments of the present invention. If an excess of loaded scaffolds with their associated capture-associated universal oligos are added to the sample, unreacted (i.e., unbound) loaded scaffolds with their associated capture-associated universal oligos should be removed prior to the hybridization reaction. The reacted capture-associated universal oligos, if any, are then analyzed.

### Sample Processing

As seen in FIG. 1, in certain embodiments, an initial step in the methods of the present invention involves obtaining and processing a biological sample containing bioterrorism target agents (e.g. antigens) from a patient. Biological samples may include, but are not limited to, sputum, amniotic fluid, whole blood, blood cells (e.g., white cells), blood serum, urine, semen, peritoneal fluid, pleural fluid, pericardial fluid, feces, ascetic fluid, spinal fluid, synovial fluid, tissue or fine needle biopsy samples, and tissue homogenates. Samples may also include sections of tissues such as frozen sections taken for histological purposes. Environmental samples may include, but are not limited to soil, air, water, organic matter, industrial samples, samples obtained from surfaces of equipment, buildings, utensils, etc.

Sample collection and preparation techniques are well known in the art (see, e.g., Tietz Textbook of Clinical Chemistry and Molecular Diagnostics, 4^{th} Ed., Chapter 2, Burtis, C. Ashwood E. and Bruns, D, eds. (2006)). In general, blood for analysis may be obtained from veins, arteries or capillaries. Venous blood is usually the specimen of choice and venipuncture is the method for obtaining this specimen. Generally, whole blood, as opposed to serum, is preferred for the present invention, as whole blood contains greater total protein. An anticoagulant must be added to the specimen during the collection procedure. A number of anticoagulants are used including heparin, EDTA, sodium fluoride, citrate, oxalate, and iodoacetate. Sputum and nasal discharge are collected directly, most commonly by swabs. A sterile Dacron® or rayon swab with a plastic shaft is preferred because calcium alginate swabs or swabs with wooden sticks may contain substances that interfere with the reactions involved in diagnosis. After collection, the swab is stored in an airtight plastic container or, preferably, immersed in liquid, such as phosphate-buffered saline or other transport medium. Environmental sample processing techniques are known in the art, (see, e.g., Tietz Textbood of Clinical Chemistry and Molecular Diagnostics, 4^{th} Ed., Chapter 2, Burtis, C. Ashwood E. and Bruns, D, eds. (2006); Chemical Weapons Convention Chemicals Analysis: Sample Collection, Preparation and Analytical Methods, Mesilaakso, M., ed., (2005); Pawliszyn, J., Sampling and Sample Preparation for Field and Laboratory, (2002); Venkatesh Iyengar, G., et al., Element Analysis of Biological Samples: Principles and Practices (1998); Drielak, S., Hot Zone Forensics: Chemical, Biological, and Radiological Evidence Collection (2004); Wells, D., High Throughput Bioanalytical Sample Preparation (Progress in Pharmaceutical and Biomedical Analysis) (2002); and Nielsen, D. M., Practical Handbook of Environmental Site Characterization and Ground-Water Monitoring (2005)).

### Capture Moieties

The capture moieties and binding partners may be any ligand associated with the bioterrorism target agent(s) to be detected. In certain embodiments, the capture moieties and/or binding partners are antibodies, preferably monoclonal antibodies, to the bioterrorism target agents. Such capture moieties and binding partners may be obtained commercially, or may be generated de novo. Tables 1a and 1b comprise exemplary lists of chemical or biological weapons that may be used in acts of terrorism. Presence or absence of bioterrorism agents may indicate whether an individual is infected with or has been exposed to a particular chemical or biological weapon, and/or to what stage infection or exposure by a particular bioterrorism agent has progressed. Chemical or biological compounds (weapons) can be used to generate antibodies for use as capture moieties or binding entities in the methods of the present invention.

Monoclonal antibodies include a natural monoclonal antibody prepared by immunizing mammals such as mice, rats, hamsters, guinea pigs or rabbits with a bioterrorism agent-associated antigen (including natural, recombinant, and chemically synthesized proteins, cell culture supernatant), or another immunogenic bioterrorism agent-associated compound, or a portion thereof; a chimeric antibody or a humanized antibody produced by recombinant technology; or a human monoclonal antibody, for example, obtained by using human antibody-producing transgenic animals. Monoclonal antibodies include those having any one of the isotypes of IgG, IgM, IgA (IgA1 and IgA2), IgD, or IgE. IgG (IgG1, IgG2, IgG3, and IgG4, preferably IgG2 or IgG4) or IgM is preferable.

Polyclonal antibodies or monoclonal antibodies can be produced by known methods. Typically, mammals, preferably, mice, rats, hamsters, guinea pigs, rabbits, cats, dogs, pigs, goats, horses, or cows, or more preferably, mice, rats, hamsters, guinea pigs, or rabbits, are immunized with a target agent along with Freund's adjuvant, if necessary. In addition, transgenic animals may be generated so as to produce an antibody derived from another animal species, such as a human antibody-producing transgenic mouse.

Specifically, a monoclonal antibody can be produced in the following manner by methods well known in the art (see, e.g., Cellular and Molecular Immunology, 5^{th} Ed., Abbas, A. and Lichtman, A. eds. (2005)). Immunizations are accomplished by introducing a chosen target agent once or several times, subcutaneously, intramuscularly, intravenously, through the footpad, or intraperitoneally, into non-human mammals. Usually, immunizations are performed once to four times every one to fourteen days after the first immunization. Antibody-producing cells are obtained from the mammal in about one to five days after the last immunization. The times and interval of the immunizations can be altered in accordance with the properties of the immunogen used.

Hybridomas that secrete a monoclonal antibody can be prepared, inter alia, by the method of Kohler and Milstein (Nature, Vol. 256, p. 495-97(19′75)) and by any other known methods or modified methods known in the art. Hybridomas are prepared by fusing the antibody-producing cells obtained from the spleen, lymph node, bone marrow, or tonsil from the non-human mammal immunized as mentioned above with mammal-derived myelomas that have no autoantibody-producing ability. For example, mouse-derived myelomas P3/X63-AG8.653 (653, ATCC No. CRL1580), P3/NSI/1-Ag4-1 (NS-1), P3/X63-Ag8.U1 (P3U1), SP2/0-Ag14 (Sp2/0, Sp2), PAI, F0, or BW5147; rat-derived myelomas 210RCY3-Ag.2.3; or human-derived myelomas U-266AR1, GM1500-6TG-Al-2, UC729-6, CEM-AGR, D1R11, or CEM-T15 can be used as a myelomas for the cell fusion. Monoclonal antibody producing cells (i.e., the hybridomas) can be screened by cultivating the cells, for example, in microtiter plates, and by measuring the reactivity of the culture supernatant by using the immunogen used for the immunization in an enzyme immunoassay such as an ELISA. The monoclonal antibodies may be produced from hybridomas by cultivating the hybridomas in vitro or in vivo such as in ascites of mice, rats, guinea pigs, hamsters, or rabbits, preferably mice or rats, and isolating the antibodies from the resulting culture supernatant or ascites fluid. In addition, monoclonal antibodies may be obtained in a large quantity by cloning a gene encoding a monoclonal antibody from a hybridoma or recombinant monoclonal antibody producing cell, generating transgenic animals such as cows, goats, sheep, or pigs in which the gene encoding the monoclonal antibody is integrated using transgenic animal generating techniques, and recovering the monoclonal antibody from the milk of the transgenic animals (see, e.g., Nikkei Science, No. 4, pp. 78-84 (1997)). Cultivating hybridomas in vitro typically is performed by using known nutrient media or nutrient media derived from known basal media. Examples of basal media are low calcium concentration media such as Ham F12 medium, MCDB153 medium, or low calcium concentration MEM medium, and high calcium concentration media such as MCDB 104 medium, MEM medium, D-MEM medium, RPMI1640 medium, ASF104 medium, or RD medium. The basal media may also contain, for example, sera, hormones, cytokines, and/or various inorganic or organic substances known in the art.

Monoclonal antibodies can be, inter alia, isolated and purified from the culture supernatant or ascites mentioned above by saturated ammonium sulfate precipitation, euglobulin precipitation, the caproic acid or caprylic acid method, ion exchange chromatography (DEAE or DE52), thiophilic resin (Clontech®), by affinity chromatography using anti-immunoglobulin column or protein A or protein G columns, or by other methods known in the art. By using the above-mentioned methods, it is possible to immunize non-human mammals, prepare and screen hybridomas producing the antibodies, and prepare the human monoclonal antibody in large quantities (see, e.g., Nature Genetics, Vol. 7, p. 13-21, 1994; Nature Genetics, Vol. 15, p. 146-156, 1997; Published Japanese Translation of PCT International Publication No. Hei 4-504365; Published Japanese Translation of PCT International Publication No. Hei 7-509137; Nikkei Science, June edition, p. 40-50, 1995; WO94/25585; Nature, Vol. 368, p. 856-859, 1994; Published Japanese Translation of PCT International Publication No. Hei 6-500233, etc.).

Monoclonal antibodies also include an antibody that comprises the heavy chain and/or the light chain in which either or both of the chains have deletions, substitutions or additions of one or several amino acids in the sequences thereof; several amino acids as referred to here means multiple amino acid residues, specifically means one to ten amino acid residues, preferably one to five amino acid residues. Such a partial modification of amino acid sequence (deletion, substitution, insertion, and addition), can be introduced into the antibody by partially modifying the nucleotide sequence encoding the amino acid sequence. The partial modification of the nucleotide sequence can be performed by the usual method of site-specific mutagenesis (see, e.g., PNAS USA, Vol. 81, p. 5662-5666 (1984)) or other methods known in the art.

An “antibody” of the present invention includes a portion of an antibody as well, including F(ab′)₂, Fab′, Fab, Fv (variable fragment of antibody), sFv, dsFv (disulfide stabilized Fv), or dAb (single domain antibody). F(ab′)₂ and Fab′ can be produced by digesting an antibody near the disulfide bonds existing between the hinge regions in each of the two H chains with a protease such as pepsin and papain, generating an antibody fragment. An IgG antibody molecule is composed of two light chains linked by disulfide bonds to two heavy chains. The two heavy chains are, in turn, linked to one another by disulfide bonds in an area known as the hinge region of the antibody. A single IgG molecule typically has a molecular weight of approximately 150-160 kD and containing two antigen binding sites. An F(ab′)₂ fragment lacks the C-terminal portion of the heavy chain constant region, and has a molecular weight of approximately 110 kD. It retains the two antigen binding sites and the interchain disulfide bonds in the hinge region, but it does not have the effector functions of an intact IgG molecule. An F(ab′)₂ fragment may be obtained from an IgG molecule by proteolytic digestion with pepsin at pH 3.0-3.5. Alternatively, papain cleaves IgG upstream of the disulfide bonds existing between the hinge regions in each of the two H chains to generate two homologous antibody fragments in which an L chain composed of V_{L} (L chain variable region) and C_{L} (L chain constant region), and an H chain fragment composed of V_{H} (H chain variable region) and C_{H}γ1 (gammal region in the constant region of H chain) are connected at their C terminal regions through a disulfide bond. Each of these two homologous antibody fragments is called Fab′.

Antibodies may be characterized by an immunoassay such as the single antibody solid phase method, two-antibody liquid phase method, two-antibody solid phase method, sandwich method, enzyme multiplied immunoassay technique (EMIT method), enzyme channeling immunoassay, enzyme modulator mediated enzyme immunoassay (EMMIA), enzyme inhibitor immunoassay, immuno-enzymometric assay, enzyme-enhanced immunoassay or proximal linkage immunoassay, all of which are described, inter alia, in Enzyme Immunoassay, 3rd Ed., Eiji Ishikawa et al., and Igakushoin eds., (1987)); or, for example, the one-pot method which is described in JP-B Hei 2-39747. However, from the standpoint of simplicity of operation and/or economical advantage, and especially when considering the clinical applicability, the sandwich method, the one pot method, the single antibody solid phase method or the two-antibody solid phase method are preferred. Most preferable is the sandwich method using a labeled antibody prepared by labeling an antibody generated with an enzyme or biotin and using an antibody-immobilized insoluble carrier prepared by immobilizing the monoclonal antibody on a multi-well microplate.

### Universal Oligo Sets and Universal Oligo Chips

The universal oligos of the present invention are oligonucleotides from a complementary or substantially complementary oligonucleotide pair, where each oligo in the pair has been rationally designed to have low complementarity to sequences that may be present in a given sample. A “universal oligo set” is a set of two or more universal oligo pairs where each oligo in the set has low complementarity to every other universal oligo in the set, with the exception of its complement. Use of universal oligo chips for detecting bioterrorism target agents has many advantages including, but not limited to the following. For example, the universal oligo chips can be used with virtually any downstream application (i.e., the front end assay can detect antibodies, antigens, chemical or biological toxins, metabolites, etc.), yet the chips have standardized hybridization conditions independent of the bioterrorism target agent. However, the universal oligo chips can be flexible as well, as different universal oligo sets may be used for different assays, where a particular universal oligo chip may have chip-associated universal oligos with melting temperatures and/or lengths of X and another universal oligo chip may have chip-associated universal oligos with melting temperatures and/or lengths of Y. In addition, the universal oligos of the present invention can be engineered to contain sequences for enzyme cleavage for use in some embodiments.

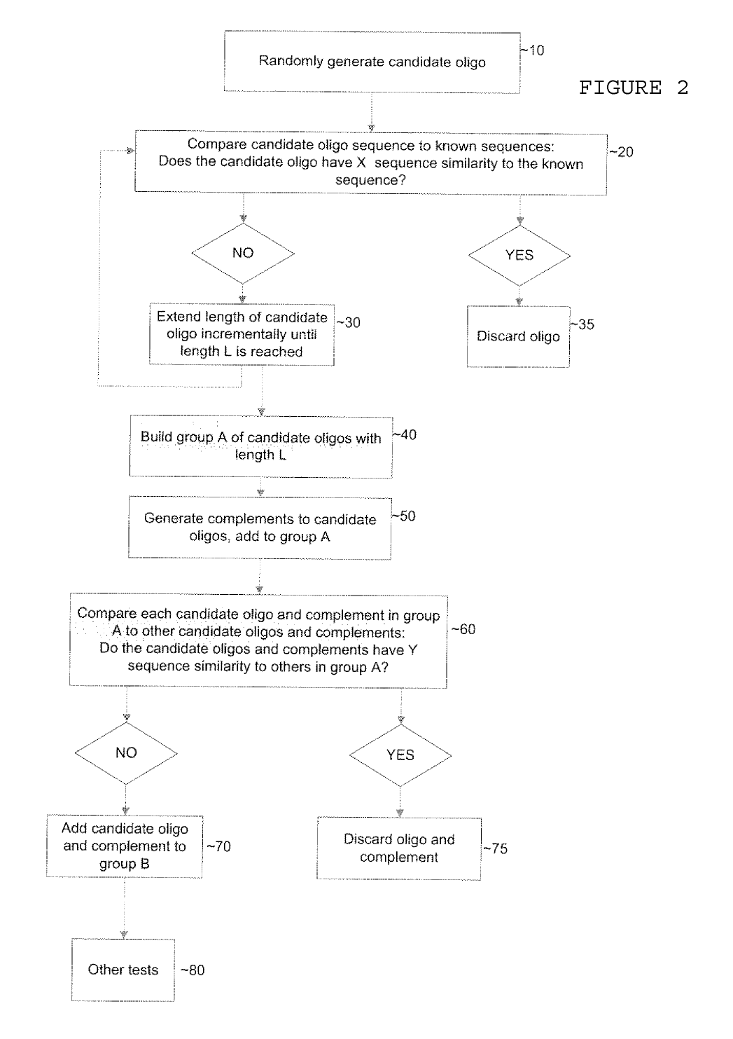

FIG. 2 is a flow compliment complement chart showing a representative non-limited overview of one embodiment for the creation of universal oligos and a universal oligo set. In step 10, candidate oligo sequences are randomly generated. Typically, such randomly generated sequences will be short, for example, 8-25 nucleotides in length. In one embodiment of the invention, all possible variations of 15-mers (consisting only nucleotides A, T, G and C) are generated and stored in a database. At step 20, each candidate sequence is compared to known sequences, typically, by comparing the candidate sequence to sequences stored in publicly-available and/or custom databases. Custom databases may be databases populated with information from publicly-available databases. Major publicly-available sequence repositories include DDBJ: DNA databank of Japan, EMBL: maintained by EMBL, and GenBank: maintained by NCBI; organelle databases include OGMP: the organelle genome megasequencing program, GOBASE: an organelle genome database, and MitoMap: a human mitochondrial genome database; RNA databases include Rfam: an RNA family database, RNA base: a database of RNA structures, tRNA database: a database of tRNAs, tRNA: tRNA sequences and genes, and sRNA: a small RNA database; comparative and phylogenetic databases include COG: phylogenetic classification of proteins, DHMHD: a human-mouse homology database, HomoloGene: a database of gene homologies across species, Homophila: a human disease to Drosophila gene database, HOVERGEN: a database of homologous vertebrate genes, TreeBase: a database of phylogenetic knowledge, XREF: a database that cross-references human sequences with model organisms; SNP, mutation and variation databases include ALPSbase: a database of mutations causing human ALPS, dbSNP: the single nucleotide polymorphism database at NCBI, and HGVbase: a human genome variation database; alternative splicing databases include ASDB: a database of alternatively spliced genes, ASAP: an alternate splicing analysis tool, ASG: an alternate splicing gallery, HASDB: a human alternative splicing database, AsMamDB: a database of alternatively spliced genes in human, mouse and rat, and ASD: an alternative splicing database at CSHL; and scores of specialized databases include ACUTS: a database of ancient conserved untranslated sequences, AGSD: an animal genome database, AmiGO: a gene ontology database, ARGH: an acronym database, BACPAC: BAC and PAC a database of genomic DNA library info, CHLC: a database of genetic markers on chromosomes, COGENT: a complete genome tracking database, COMPEL: a database of composite regulatory elements in eukaryotes, CUTG: a codon usage database, dbEST: a database of expressed sequences or mRNA, dbGSS: genome survey sequence database, dbSTS: a database of sequence tagged sites (STS), DBTSS: a database of transcriptional start sites, DOGS: a database of genome sizes, EID: the exon-intron database, Exon-Intron: an exon-intron database, EPD: a eukaryotic promoter database, FlyTrap: a HTML-based gene expression database, GDB: the genome database, GeneKnockouts: a database of gene knockout information, GENOTK: a human cDNA database, GEO: a gene expression omnibus NCBI, GOLD: a database of information on genome projects around the world, GSDB: the Genome Sequence DataBase, HGI: TIGR human gene index, HTGS: a database of genomic sequences at NCBI, IMAGE: a database of the largest collection of DNA sequences clones, IMGT: a database of the international ImMunoGeneTics information system, LocusLink: single query interface to sequence and genetic loci, TelDB: ae telomere database, MitoDat: a database of mitochondrial nuclear genes, Mouse EST: a database with information from the NIA mouse cDNA project, MPSS: searchable databases of several species, NDB: a nucleic, acid database, NEDO: a human cDNA sequence database, NPD: a nuclear protein database, PLACE: a database of plant cis-acting regulatory DNA elements, RDP: a ribosomal database, RDB: a receptor database at NIHS, Japan, Refseq: the NCBI reference sequence project, RHdb: a database of radiation hybrid physical map of chromosomes, SpliceDB: database of canonical and non-canonical splice site sequences, STACK: a database of consensus human EST database, TAED: the adaptive evolution database, TIGR: curated databases of microbes, plants and humans, TRANSFAC: the transcription factor database, TRRD: a transcription regulatory region database, UniGene: a database of cluster of sequences for unique genes at NCBI, and UniSTS: a database of non-redundant STS.

For sequence comparison, known sequences act as reference sequences to which the candidate sequences are compared. When using a sequence comparison algorithm, known and candidate sequences are input into a computer, subsequence coordinates are designated if appropriate, and sequence algorithm program parameters are designated. The sequence comparison algorithm then calculates the percent sequence identity or regions of sequence identity for the candidate sequence relative to the known reference sequence, based on the designated program parameters.

In the present invention, universal oligos are designed for detection of bioterrorism target agents which in turn indicates the presence of a chemical or biological weapon that characterizes an act of bioterrorism. As such, candidate sequences are screened against sequences from mammals, viruses and bacteria contained in a custom database containing information from publicly-available databases.

The determination of percent identity between two or more sequences can be accomplished using a mathematical algorithm. Preferred, non-limiting examples of such mathematical algorithms include, inter alfa, the algorithm of Myers and Miller (1988); the search-for-similarity-method of Pearson and Lipman (1988); and that of Karlin and Altschul (1993). Preferably, computer implementations of these mathematical algorithms are utilized. Such implementations include, but are not limited to: CLUSTAL in the PC/Gene program (available from Intelligenetics, Mountain View, Calif.); the ALIGN program (Version 2.0), GAP, BESTFIT, BLAST, FASTA, Megalign (using Jotun Hein, Martinez, Needleman-Wunsch algorithms), DNAStar Lasergene (see www.dnastar.com) and TFASTA in the Wisconsin Genetics Software Package, Version 8 (available from Genetics Computer Group (GCG), 575 Science Drive, Madison, Wis., USA). Alignments using these programs can be performed using the default parameters or parameters selected by the operator. The CLUSTAL program is well described by Higgins. The ALIGN program is based on the algorithm of Myers and Miller; and the BLAST programs are based on the algorithm of Karlin and Altschul. Software for performing BLAST analyses is publicly available through the National Center for Biotechnology Information (http://www.ncbi.nlm.nih.gov/).

If a candidate sequence is found to have sequence similarity above a given limit (however this limit is defined, e.g., X % homology overall or a percentage over a Y basepair stretch of a sequence) during the screening against known sequences, the candidate sequence will be discarded (step 35). If a candidate sequence is found to have sequence homology below a given limit during the screening against known sequences, the candidate sequence will be extended by one or more nucleotides (step 30) and will go through the screening process again.

In a preferred embodiment, the candidate sequence will be extended by one nucleotide at a time (step 30), but will be extended by each of A, T, G and C. For example, if candidate sequence XXXXXXXXXXXXXXX is determined to have sequence homology below the given limit, candidate sequence XXXXXXXXXXXXXXX will then be extended by one nucleotide four times, that is, candidate sequence XXXXXXXXXXXXXXX will be extended to candidate sequence XXXXXXXXXXXXXXXA, candidate sequence XXXXXXXXXXXXXXXT, candidate sequence XXXXXXXXXXXXXXXG and candidate sequence XXXXXXXXXXXXXXXC and each of these candidate sequences will be screened as described previously (step 20). The process can be continued until a desired length L is achieved. Once a candidate sequence of desired length L is found, it is placed in a group A of candidate sequences (step 40), and these candidate sequences are used to build a universal oligo set. Though the sequences above are written in conventional 5′-3′ mode, extension can take place from either end.

In building a universal oligo set, sequences complementary to the candidate sequences can be generated and added to the candidate sequences in group A (step 50). At step 60, each candidate sequence and complement in group A are compared to each other candidate sequence and each other complement to determine the extent of sequence similarity (however “sequence similarity” is defined). If a candidate or complement sequence is found to have sequence similarity above a given limit (again, however “sequence similarity” is defined) during the screening at step 60, the candidate sequence and its complement will be discarded (step 75). If it is determined that a candidate sequence and its complement are found to have sequence homology below a given limit during the screening at step 60, the candidate sequence and complement will be added to a group B (step 70). The candidate and complementary sequences in group B may then be subjected to further screening (step 80), using various parameters such as melting temperature (Tm), existence of duplexes, specificity of hybridization, existence of a GC clamp, existence of hairpins, existence of sequence repeats, dissociation minimum for a 3′ dimer, dissociation minimum for the 3′ terminal stability range, frequency threshold, or maximum length of acceptable dimers and the like.

The universal oligos can be 1 to 10000 bases in length, preferably 10 to 1000 bases in length, more preferably 10-500 bases in length and more preferably about 25 to about 100 bases in length. Additionally, the universal oligos may be DNA, RNA or PNA (peptide nucleic acid) and can include non-naturally occurring subunits, sequences and/or moieties. PNA includes peptide nucleic acid analogs. The backbones of PNA are substantially non-ionic under neutral conditions, in contrast to the highly charged phosphodiester backbone of naturally occurring nucleic acids. This results in, inter alia, two advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tm for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration. This can be advantageous in certain embodiments, as a reduced salt hybridization solution has a lower Faradaic current than a physiological salt solution (in the range of 150 mM).

### Conjugation

Conjugation of the capture-associated universal oligos to the capture moieties may be performed in numerous ways, providing it results in a capture moiety possessing both epitope-specific binding to capture the bioterrorism target agent as well as providing it does not restrict nucleic acid hybridization functionalities in embodiments where a cleavage is not performed, to allow detection of the bound bioterrorism target agent. For example, nucleic acid-antibody conjugates can be synthesized by using heterobifunctional cross-linker chemistries to covalently attach single-stranded DNA labels through amine or sulfhydryl groups on an antibody to create a capture agent of the invention (see, e.g., Hendricksen E R, Nucleic Acids Res., 23(3):522-9 (1995)). In another example, covalent single-stranded DNA-strepavidin conjugates, capable of hybridizing to complementary surface-bound oligonucleotides, are utilized for the effective immobilization of biotinylated antibodies. See, e.g., Niemeyer C M, et al., Nucleic Acids Res.; 31(16):90 (1995). Many other nucleic acid molecular conjugates are described, for example, in Heidel J., et al., Adv Biochem Eng Biotechnol.; 99:7-39 (2005). Additional methods of creating antibody-oligo conjugates, both those existing and under development, will be apparent to one skilled in the art upon reading the present disclosure, and such methods are intended to be captured within the methods of the invention.

An alternative to directly conjugating the capture-associated universal oligo to the capture moiety, an as described in detail herein, some embodiments of the present invention utilize scaffolds to which the capture-associated universal oligos and the capture moieties are conjugated or otherwise associated. Scaffolds can be comprised of any substrate capable of supporting oligonucleotides and capture moieties. In one embodiment, the scaffold is comprised of a nanoparticle. Nanoparticles useful in the practice of the invention include metal (e.g., gold, silver, copper and platinum), semiconductor (e.g., CdSe, CdS, and CdS or CdSe coated with ZnS) and magnetic (e.g., ferromagnetite) colloidal materials. Other nanoparticles useful in the practice of the invention include ZnS, ZnO, TiO₂ AgI, AgBr, HgI₂, PbS, PbSe, ZnTe, CdTe, In₂S₃, In₂Se₃, Cd₃P₂, Cd₃As₂, InAs, and GaAs. The size of the nanoparticles is preferably from about 5 nm to about 150 nm (mean diameter), more preferably from about 5 to about 50 nm, most preferably from about 10 to about 30 nm. The nanoparticles may also be rods.

Methods of making metal, semiconductor and magnetic nanoparticles are well-known in the art. See, e.g., Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Methods of making ZnS, ZnO, TiO₂ AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshaysky et al., J. Am. Chem. Soc., 112, 9438 (1990); Ushida et al., J. Phys. Chem., 95, 5382 (1992). Suitable nanoparticles are also commercially available from, e.g., Ted Pella, Inc. (gold), Amersham Corporation (gold) and Nanoprobes, Inc. (gold).

Loaded scaffolds are made by attaching oligonucleotides and capture moieties onto a suitable substrate. Methods of attaching or associating oligonucleotides and capture moieties such as antibodies to substrates such as gold particles are well known in the art. A brief example of such methods using gold nanoparticles for the scaffold is as follows: Gold colloid of a particle size suited to the needs of the user is prepared using well known methods (Beesley J., (1989), “Colloidal Gold A new perspective for cytochemical marking”. Royal Microscopical Society Handbook No 17. Oxford Science Publications. Oxford University Press). In such a method, 100 ml of 0.01% gold chloride solution is adjusted to pH 9.0. Antibody solution is prepared by making a 0.1 μg/μl solution of antibody in 2 mM borax and dialyzing for at least 4 hours against 1 liter of borax at pH 9.0. The antibody solution is centrifuged at 100,000 g for 1 hour at 4° C. immediately prior to use. The dialyzed and centrifuged antibody solution (0.1 μg/μl) is adjusted to pH 9.2, and appropriate amount of antibody solution is then added drop wise to 100 ml of the gold solution while stirring rapidly. After 5 minutes, 5 ml of filtered 10% BSA at pH 9.0 is added to the antibody-gold particle solution and stirred gently for 10 minutes. The solution is then purified by centrifugation to form an antibody-gold particle scaffold conjugate.

Oligonucleotides can be attached to the antibody-gold particle scaffold through the use of functionalized chemical groups such as alkanethiol, alkylthiol, or other functionalized thiols attached to either terminal end of the oligonucleotide. Methods for attaching oligonucleotides to antibody-modified gold particles are well known in the art. An example of such preparation is as follows: alkylthiol functionalized oligonucleotides are reacted with an appropriate amount of antibody-gold particle scaffold solution for 16 hours and then stabilized with salt to 0.1M NaCl. 10% BSA is then added to the solution for 30 minutes to stabilize the gold particle scaffolds. This solution is then purified via centrifugation at 20,000 g for one hour at 4° C., the supernatant is removed, and the centrifugation is repeated. 0.1 M NaCl/0.01M phosphate buffer solution at pH 7.4 is used to resuspend the pellet. The loaded scaffold in the solution comprises antibodies and oligonucleotides associated with a gold particle scaffold.

Other nanoparticles may be used as substrates for oligonucleotide binding, and methods for binding oligonucleotides to such substrates is well known in the art. Briefly, the following references describe other substrates and linking agents that can be used to bind oligonucleotides to nanoparticles: Nuzzo et al., J. Am. Chem. Soc., 109, 2358 (1987) (disulfides for oligo attachment on gold); Allara and Nuzzo, Langmuir, 1, 45 (1985) (carboxylic acids for oligo attachment on aluminum); Allara and Tompkins, J. Colloid Interface Sci., 49, 410-421 (1974) (carboxylic acids for oligo attachment on copper); Iler, The Chemistry Of Silica, Chapter 6, (Wiley 1979) (carboxylic acids for oligo attachment n silica); Timmons and Zisman, J. Phys. Chem., 69, 984-990 (1965) (carboxylic acids for oligo attachment on platinum); Soriaga and Hubbard, J. Am. Chem. Soc., 104, 3937 (1982) (aromatic ring compounds for oligo attachment on platinum); Hubbard, Acc. Chem. Res., 13, 177 (1980) (sulfolanes, sulfoxides and other functionalized solvents for oligo attachment on platinum); Hickman et al., J. Am. Chem. Soc., 111, 7271 (1989) (isonitriles for oligo attachment on platinum); Proupin-Perez et al., Nucleosides Nucleotides and Nucleic Acids, 24, 1075 (2005) (maleimides for oligo attachment on silica); Eltekova and Eltekov, Langmuir, 3, 951 (1987) (aromatic carboxylic acids, aldehydes, alcohols and methoxy groups for oligo attachment on titanium dioxide and silica); Lec et al., J. Phys. Chem., 92, 2597 (1988) (rigid phosphates for oligo attachment on metals); Jung et al., Langmuir 20, 8886 (2004) (carboxylic acids for oligo attachment on carbon nanotubes).

Other particles capable of binding oligonucleotides include polymeric particles (such as polystyrene particles, polyvinyl particles, acrylate and methacrylate particles), glass particles, latex particles, Sepharose beads and other like particles. The conjugation of these particles with oligonucleotides is well known in the art. Functional groups used to mediate the transfer of oligonucleotides onto the particle include carboxylic acids, aldehydes, amino groups, cyano groups, ethylene groups, hydroxyl groups, mercapto groups, and other similar functional groups. The following references describe the transfer of oligonucleotides onto these particles: Chrisey et al., Nucleic Acids Research, 24, 3031-3039 (1996) (glass) and Charreyre et al., Langmuir, 13, 3103-3110 (1997), Fahy et al., Nucleic Acids Research, 21, 1819-1826 (1993), Elaissari et al., J. Colloid Interface Sci., 202, 251-260 (1998), Kolarova et al., Biotechniques, 20, 196-198 (1996) and Wolf et al., Nucleic Acids Research, 15, 2911-2926 (1987).

Magnetic, polymer-coated magnetic, and semiconducting particles can also be used as substrates for attachment of oligonucleotides. The conjugation of these particles with oligonucleotides is well known in the art. For reference, see Chan et al., Science, 281, 2016 (1998); Bruchez et al., Science, 281, 2013 (1998); Kolarova et al., Biotechniques, 20, 196-198 (1996). Use of functionalized polymer-coated magnetic particles (Fe₃O₄) are well known in the art and available from Dynal (Dynabeads™) and silica-coated magnetic Fe₃O₄ nanoparticles may be modified (Liu et al., Chem. Mater., 10, 3936-3940 (1998)) using well-developed silica surface chemistry (Chrisey et al., Nucleic Acids Research, 24, 3031-3039 (1996)) and employed as magnetic probes as well.

### Electrochemical Biosensors for Use in the Present Invention

Various biosensors known to those skilled in the art may be used in the present invention to detect the presence and/or abundance of a bioterrorism target agent in a sample. One general type of biosensor for use in the present invention employs an electrode surface in combination with current or impedance measuring elements for detecting a change in current or impedance in response to the presence of a detection moiety brought within an appropriately close distance (“proximity”) of the electrode to enable a distinct and reproducible redox reaction. The distance necessary to achieve a distinct and reproducible redox reaction, and thus electrochemical measurement of binding, will vary depending upon the nature of the detection moiety and the properties of the electrode surface. Determining the necessary proximity of a detection moiety to effect the desired reaction will be well within the skill of one in art upon reading the present disclosure.

The biosensors for use with the present invention can be produced in a disposable format, intended to be used for a single detection experiment or a series of detection experiments and then discarded.

Certain embodiments of the invention further provide an electrode assembly including both a detection electrode and a reference electrode required for electrochemical detection. Conveniently, the electrode assembly could be provided as a disposable unit comprising a housing or holder manufactured from an inexpensive material equipped with electrical contacts for connection of the detection electrode and reference electrode.

Electrochemical biosensors capable of detecting and quantifying bioterrorism target agents in a sample, such as those described and used in the present invention, offer many advantages over strictly biochemical assay formats. First, electrochemical biosensors may be produced, using conventional microchip technology, in highly reproducible and miniaturized form, with the capability of placing a large number of biosensor elements on a single substrate (e.g., see U.S. Pat. Nos. 5,200,051 and 5,212,050). Secondly, because small electrochemical signals can be readily amplified (and subjected to various types of signal processing if desired), electrochemical biosensors have the potential for measuring minute quantities of a target agent, and proportionately small changes in target agent levels. Importantly, electrochemical biosensors may offer this exquisitely sensitive detection at a lower cost than currently available assay methods.

The preferred biosensor for use in the present invention comprises a conventional electrode with a modified surface allowing oligo attachment, and thus the description herein is focused on the use of such an electrode. Other biosensor systems, however, may be utilized in the assay methods of the invention, as will be apparent to one skilled in the art upon reading this disclosure, and these are intended to be encompassed within the present invention. Examples of other biosensors that may be utilized with the present invention include, but are not limited to biosensors disclosed, for example, in U.S. Pat. No. 5,567,301; biosensors based on surface plasmon resonance (SPR), as described in U.S. Pat. No. 5,485,277; and biosensors that utilize changes in optical properties at a biosensor surface, e.g., as described in U.S. Pat. No. 5,268,305.

In accordance with one embodiment of the present invention, one oligo of a universal oligo pair, the chip-associated universal oligo, is immobilized (directly or indirectly) onto an electrochemical surface. Although a metal electrode (e.g., gold, aluminum, platinum, palladium, rhodium, ruthenium, any metal or other material having a free electron in its outer most orbital) is preferably employed as the surface for immobilizing the chip-associated universal oligo, other surfaces such as photodiodes, thermistors, ISFETs, MOSFETs, piezo elements, surface acoustic wave elements, and quartz oscillators may also be employed. Preferred electrodes are known in the art and include, but are not limited to, certain metals and their oxides, including gold; platinum; palladium; silicon; aluminum; titanium, metal oxide electrodes including platinum oxide, titanium oxide, tin oxide, indium tin oxide, palladium oxide, silicon oxide, aluminum oxide, molybdenum oxide (Mo₂,O₆), tungsten oxide (WO₃) and ruthenium oxides; carbon (including glassy carbon electrodes, graphite, pyrolytic graphite, carbon fiber, and carbon paste); and semiconductor electrodes, such as Si, Ge, ZnO, CdS, TiO₂ and GaAs. Preferred electrodes include gold, silicon, platinum, carbon and metal oxide electrodes, with gold being particularly preferred. The electrode may also be covered with conductive compounds to enhance the stability of the electrodes immobilized with probes or nonconductive (e.g., insulative materials). Monomolecular films or biocompatible materials may also be employed to coat or partially coat the electrodes.

The electrodes described herein are presumed to be a flat surface, which is only one of the possible conformations of the electrode. The conformation of the electrode depends upon the detection method employed. For example, flat planar electrodes may be preferred for electrochemical detection methods, thus requiring addressable locations for synthesis and/or detection. In a preferred embodiment, the detection electrodes are formed on a glass or polymer substrate. The discussion herein is generally directed to the formation of gold electrodes, but as will be appreciated by those in the art, other electrodes can be used as well. The substrate can comprise a wide variety of materials, as will be appreciated by those in the art, with glass, polymers and printed circuit board (PCB) materials being particularly preferred. Thus, in general, the suitable substrates include, but are not limited to, fiberglass, Teflon, ceramics, glass, silicon, mica, plastic (including acrylics, polystyrene and copolymers of styrene and other materials, Mylar, polypropylene, polyethylene, polybutylene, polycarbonate, polyurethanes, Teflon™, and derivatives thereof, etc.), GETEK (a blend of polypropylene oxide and fiberglass), and other materials typically employed and readily known to those of ordinary skill in the art.

As is generally known in the art, one or a plurality of layers may be used, to make either “two-dimensional” (e.g. all electrodes and interconnections in a plane) or “three dimensional” substrates. Three-dimensional systems frequently rely on the use of drilling or etching, followed by electroplating with a metal such as copper, such that the “through board” interconnections are made, or comprise porous structures similar to, inter alia, xeolites in structure.

The electrode for use in the present invention preferably comprises a mixed monolayer on the conductive surface of the electrode, the monolayer comprising both anchoring groups conjugated to chip-associated oligos and diluent groups which serve as an insulator on the electrode surface. Depending on the length, sequence, and secondary structure of the oligo, specific spacing of the anchoring groups and the diluent groups can be designed to maximize interaction capabilities. For example, it can be advantageous to have only small sub-monolayer amounts of the chip-associated oligo present on the surface to enhance the hybridization properties of the chip-associated oligos with the capture-associated universal oligos, particularly if the capture-associated universal oligos are still attached to their capture moieties. Also, several different chip-associated oligos can be introduced at the same time into the monolayer to create a monolayer with detection capabilities for multiple target agents.

One specific method for enhancing the binding of oligos to a biosensor is thus utilizing a specific ratio of anchoring groups attached to the chip-associated oligos (together referred to as “anchoring group complexes”) and diluent groups in the monolayer on the electrode. The ratio of bound anchoring group complexes and diluent agents on the electrode can be designed to optimize the access of the electron-associated oligo to any capture-associated universal oligo present in an assay. The ratio is preferably designed to be a concentration of the chip-associated oligo that will limit binding interference due to conformational interactions between multiple chip-associated oligos. Biosensors with specific concentrations of the diluent agents and the anchoring group complexes will enhance the availability of the chip-associated oligos for binding to the capture-associated universal oligos while maintaining the insulating monolayer on the electrode. The final ratio of the components of the biosensor is preferably designed to create uniform monolayers with evenly distributed anchoring group complexes and diluent groups. The ratio of anchoring group complexes and diluent groups is preferably designed to maximize access to the chip-associated oligos, and to provide an enhancement of detection of the hybridization of capture-associated universal oligos to the biosensor.

In determining the appropriate concentration of the components to be used in depositing the monolayer on the conductive surface, a number of practical issues must be considered. For example, great differences in chain length or size of the chip-associated oligo on anchoring group complexes can lead to preferential adsorption of the diluent groups. This can also lead to formation of islands of anchoring group complex surrounded by diluent agents. Bain C D, Evall J, Whitesides G M. J Am Chem Soc; 111: 7155-7164 (1989); Bain C D, Whitesides G M. J Am Chem Soc; 111: 7164-7175 (1989). In addition, as a general rule, the SAM composition will not be deposited on the surface in the same concentration ratio as in the preparation solution. Characterization of the SAM surface with an analytical tool, e.g., infrared spectroscopy, ellipsometry, studies of wetting by different liquids, x-ray photoelectron spectroscopy, electrochemistry, and scanning probe measurements, thus may be necessary to calibrate the mixing ratio can be used to determine the most appropriate ratio for specific anchoring agent complexes, as will be apparent to one skilled in the art upon reading the present specification. For example, in certain embodiments, the electrostatic repulsion between DNA strands may help suppress island formation; in other embodiments, such as those employing peptide oligos, the electrostatic repulsion will be reduced and may not serve to prevent this phenomenon.

The detection of the capture-associated universal oligo using an electrode is based on an electrochemical reaction on the conductive detection surface, which requires that electrons tunnel from the electron donor through the insulating monolayer. Because the primary mechanism by which electrochemical detection takes place is via “through-bond” electron tunneling rather than interchain electron tunneling, the composition of the linkage of the oligo complex will have a significant effect on the electron transfer rate. To achieve the most accurate and efficient signal, both the anchoring group and the diluent group, which forms the insulator composition, are selected to maximize the ratio of specific current to non-specific, or “leakage” current. The efficiency of the tunneling can thus be controlled by manipulation of the molecules which comprise the monolayer.

The insulating properties of the monolayer film will thus depend upon the chemical composition of the molecules forming the monolayer. For example, the properties of an alkane thiol versus an ether thiol can significantly change the rate constant, with the rate constant through the alkane linker shown on an order of four times faster than through the ether linker. The composition of the non-complexed SAM components can impact on the overall electron transfer rate, though not as significantly as with the linkage of the oligo complex. In this case, non-complexed ether thiol molecules (“diluent molecules”) will reduce the overall rate constant slightly versus their alkane counterparts. Ether linkages are more highly insulating than alkane groups, presumably because of an energy effect.

For use in the assays of the invention, the electrodes can be designed so that the anchoring group and the diluent group have the same chemical composition, e.g., both are alkane thiols, or alternatively the anchoring group and the diluent group have different chemical compositions, e.g., the anchoring group is an alkane thiol and the diluent group is an ether thiol.

In a particular embodiment, the anchoring group comprises a hydrocarbon component (e.g., an alkylthiol) and a polyethylene glycol group, which will impart a greater level of hydrophilicity to the biosensor and provide additional flexibility to the chip-associated oligo linkage. The hydrocarbon component would be roughly the same length as the alkylthiol diluent molecule, promoting tight packing and perhaps more importantly discouraging so-called “phase separation” into DNA-rich and DNA-poor domains. The PEG component would serve as a hydrophilic “vertical” spacer to create further distance between the oligo and the monolayer surface. For example, synthesis of the biosensor SAM-forming molecules can comprise at least one anchoring group comprising an alkylthiol group linked to a PEG component and an oligo, and at least one substantially hydrophobic alkane diluent group. When provided within suitable (polar) carrier solvents, these molecules are able to self-assemble on the electrode. The characteristics of the hydrophilic domain (e.g., length of the PEG backbone) and the concentration of the anchoring group complex and the diluent group can be independently varied.

Since the diluent is likely to be the more reactive component, the solution compositions used to create the monolayer are biased in favor of the DNA-bearing component, and may range from a 1:1 to a 100:1 ratio of anchoring group complexes to diluent agent. In the methods of the invention related to manufacture of the biosensor, the components of the monolayer may be introduced in a single solution, in two solutions used simultaneously, or introduced sequentially to promote the adherence of the anchoring group complex e.g., the anchoring group complex solution is allowed to bind to the conductive surface for a period before introduction of the solution containing the diluent groups.

The overall concentration of the diluent group and anchoring group complexes, as well as the length of the molecules used in creating the self-assembled monolayer, will also determine the binding angle of the components of the monolayer, which affects both the thickness of the monolayer and the efficiency of the electron tunneling from the detector moiety to the electrode. The optimum binding angle can be designed based on the predicted thickness of the monolayer versus the length of the molecules in the SAM. The desired binding angle can be calculated and the monolayer appropriately designed to maximize the ratio of specific current to leakage current.

In a specific embodiment, the monolayer is composed of diluent groups and anchoring groups of 6-22 carbon atom chains attached at their proximal ends to the detection surface. In certain embodiments, the monolayer may be composed of anchoring group complexes and diluent agents attached at their proximal ends to the detection surface by a thiol linkage at a molecular density of about 3 to 5 chains/nm². In one aspect of this embodiment, the anchoring agent is present on the electrode in approximately a 10:1 to a 50:1 ratio of anchoring group complexes to diluent agent.

In one particular embodiment, the conductive detection surface of the biosensor is gold. Alkanethiol SAMs adsorbed on gold present several advantages. First, gold is a relatively inert metal that resists oxidation and atmospheric contamination fairly well Chesters M A, Somorjai G A. Surf Sci, 52: 21-28 (1975). Second, gold has a strong specific interaction with sulfur, providing a reproducible method for adhering the thiol groups to the surface of a gold detection surface Nuzzo R G. Fusco F A, Allara D L. J Am Chem Soc, 109: 2358-2368 (1987). The predictable binding of sulfur to gold allows the formation of tightly packed monolayers even in the presence of many other functional groups Bain C D, Troughton E B, Tao Y-T, Evall J, Whitesides G M, Nuzzo R G. J Am Chem Soc, 111: 321-335 (1989). Third, long-chain alkanethiols form a densely packed, crystalline or liquid-crystalline monolayer due to strong molecular interactions (van der Waals forces) between the long carbon chains Strong L, Whitesides G M. Langmuir, 4: 546-558 (1988).

In one embodiment, the anchoring group and the diluent group are both terminated with a thiol group that will interact directly with the conductive detection surface, e.g., the electrode. By mixing two or more differently terminated thiols in the preparation solution, a mixed monolayer can be prepared on the conductive surface as a mixed SAM. The relative proportion of the different groups in the assembled SAM will depend upon several parameters, like the mixing ratio in solution, the alkane chain lengths, the solubilities of the thiols in the solvent used, and the properties of the chain-terminating groups.

Preparing a SAM of alkanethiol molecules is a fairly simple process. A gold-coated substrate is immersed in a dilute solution of the alkanethiol in ethanol and a monolayer spontaneously assembles on the surface of the substrate over a period of 1-24 hours. A disordered monolayer is formed within a few minutes, during which time the thickness reaches 80-90% of its final value. Over the next several hours, van der Waals forces on the carbon chains help pack the long alkanethiol chains into a well-ordered, crystalline layer (Dubois L H, Nuzzo R G. Annu Rev Phys Chem, 43: 437-463 (1992)). In this process contaminants are replaced, solvents are expelled from the monolayer, and defects are reduced while packing is enhanced by lateral diffusion of the alkanethiols (Bain C D, Troughton E B, Tao Y-T, Evall J, Whitesides G M, Nuzzo R G. J Am Chem Soc, 111: 321-335 (1989)).

The resulting monolayers assemble with the alkanethiolates in a hexagonal-packing arrangement. This chain spacing is larger than the ideal distance needed to maximize van der Waals interactions between the chains. Therefore, a natural tilt develops 30° from the normal surface, maximizing molecular interactions between carbon chains as they pack into their final crystalline monolayer. The importance of van der. Waals interactions between the chains is also seen when one considers the chain length. In general, the longer the chain length, the more ordered the monolayer Bain C D, Evall J, Whitesides G M. J Am Chem Soc, 111: 7155-7164 (1989); Holmes-Farley S R, Bain C D, Whitesides G. Langmuir, 4: 921-937 (1988).

Contact angle measurements further confirm that alkanethiolate SAMs are very dense and that the contacting liquid only interacts with the topmost chemical groups. Reported advancing contact angles with water range from 111° to 115° for hexadecanethiolate SAMs. At the other end of the wettability scale, there are hydrophilic monolayers, e.g., SAMs of 16-mercaptohexadecanol (HS(CH₂)₁₆OH), that display water contact angles of <10°. These two extremes are only possible to achieve if the SAM surfaces are uniform and expose only the chain-terminating group at the interface. Mixed SAMs of CH₃— and OH-terminated thiols can be tailor-made with any wettability (in terms of contact angle) between these limiting values.

Another SAM preparation method is the formation of two-component molecular gradients, as first described by Liedberg and Tengvall (Langmuir, 11:3821 (1995). By cross-diffusion of two differently terminated thiols through an ethanol-soaked polysaccharide gel (Sephadex LH-20, a chromatography material) that is covering the gold substrate, a continuous gradient of 10-20 mm length may be formed. Ethanol solutions of each of the two thiols are simultaneously injected into two glass filters at opposite ends of the gold substrate. The presence of the polysaccharide gel makes the diffusion and the thiol attachment to the surface slow enough for a gradient of macroscopic dimension (several mm) to form.

The chip-associated oligos are functionalized with the anchoring group to form the anchoring group complex which is attached to the detection surface, e.g., an electrode surface. Such methods are well known in the known in the art. For instance, nucleotides functionalized with thiols at their 3′-termini or 5′-termini readily attach to gold nanoparticles. See Whitesides, Proceedings of the Robert A. Welch Foundation 39th Conference on Chemical Research Nanophase Chemistry, Houston, Tex., pages 109-121 (1995). See also, Mucic et al. Chem. Commun. 555-557 (1996) (describes a method of attaching 3′ thiol DNA to flat gold surfaces). The thiol method can also be used to attach oligos to other metal, semiconductor and magnetic colloids. Other functional anchoring groups for attaching oligos to solid surfaces include phosphorothioate groups (see, e.g., U.S. Pat. No. 5,472,881 for the binding of oligonucleotide-phosphorothioates to gold surfaces), substituted alkylsiloxanes (see, e.g. Burwell, Chemical Technology, 4, 370-377 (1974), and Matteucci and Caruthers, J. Am. Chem. Soc., 103, 3185-3191 (1981) for binding of oligos to silica and glass surfaces, and Grabar et al., Anal. Chem., 67, 735-743 for binding of aminoalkylsiloxanes and for similar binding of mercaptoaklylsiloxanes). Oligos terminated with a 5′ thionucleoside or a 3′ thionucleoside may also be used for attaching oligos to solid surfaces. Oligos may be attached to the electrode using other known binding partners, e.g., using biotin-labeled oligos and strepavidin-gold conjugate colloids; the biotin-strepavidin interaction attaches the colloids to the oligonucleotide. Shaiu et al., Nuc. Acids Res., 21, 99 (1993). The following references describe other anchoring groups which may be employed to attach oligos to electrode surfaces: Nuzzo et al., J. Am. Chem. Soc., 109, 2358 (1987) (disulfides on gold); Allara and Nuzzo, Langmuir, 1:45 (1985) (carboxylic acids on aluminum); Allara and Tompkins, J. Colloid Interface Sci., 49, 410-421 (1974) (carboxylic acids on copper); Iler, The Chemistry Of Silica, Chapter 6, (Wiley 1979) (carboxylic acids on silica); Timmons and Zisman, J. Phys. Chem., 69, 984-990 (1965) (carboxylic acids on platinum); Soriaga and Hubbard, J. Am. Chem. Soc., 104, 3937 (1982) (aromatic ring compounds on platinum); Hubbard, Acc. Chem. Res., 13, 177 (1980) (sulfolanes, sulfoxides and other functionalized solvents on platinum); Hickman et al., J. Am. Chem. Soc., 111, 7271 (1989) (isonitriles on platinum); Maoz and Sagiv, Langmuir, 3, 1045 (1987) (silanes on silica); Maoz and Sagiv, Langmuir, 3, 1034 (1987) (silanes on silica); Wasserman et al., Langmuir, 5, 1074 (1989) (silanes on silica); Eltekova and Eltekov, Langmuir, 3, 951 (1987) (aromatic carboxylic acids, aldehydes, alcohols and methoxy groups on titanium dioxide and silica); Lec et al., J. Phys. Chem., 92, 2597 (1988) (rigid phosphates on metals).

In one embodiment of the invention, a film of electroconductive polymer is deposited onto the internal surface of an electrically conductive electrode by electrochemical synthesis from a monomer solution introduced onto the structure. Electrodeposition of the electroconductive polymer film can be carried out, e.g., according to the methods disclosed in U.S. Pat. No. 6,770,190 to Milanovski, et al. In such an exemplary method, a solution containing monomers, a polar solvent and a background electrolyte are used for deposition of the polymer.

Electroconductive polymers can be doped at the electrochemical synthesis stage to modify the structure and/or conduction properties of the polymer. A typical dopant anion is sulphate (SO₄²⁻), which is incorporated during the polymerization process to neutralize any positive charge on the polymer backbone. Sulphate is not readily released by ion exchange and thus helps to maintain the structure of the polymer. Dopant anions having maximum capability for ion exchange with the solution surrounding the polymer can be used to increase the sensitivity of the electrodes. This is accomplished by using a salt with anions having a large ionic radius as the background electrolyte when preparing the electrochemical polymerization solution, e.g., sodium dodecyl sulphate and dextran sulphate. The concentration of these salts in the electrochemical polymerization solution is varied according to the type of test within the range 0.005-0.05 M.

In another embodiment, the electroactive polymer is introduced to the surface of the electrode via an introduced functional group, e.g., a sulfide, disulfide, amino, amide, amido, a carboxyl, a hydroxyl, carbonyl, oxide, phosphate, sulfate, aldehyde, keto, thiol, ester or mercapto groups. Other highly reactive functional groups may also be employed using methods readily known to those of ordinary skill in the art. For example, polymers with an associated thiol group can be bound directly to a gold or platinum surface. This embodiment may be preferable for the use of more complex polymers that are difficult to synthesize using monomer deposition.

In a specific embodiment, the biosensor used for detection of the target agent comprises: (a) a conductive surface, e.g., gold, platinum, or carbon, that functions as an electrode; (b) an insulating polymer that is uniformly distributed on the electrode; (c) an adapter molecule associated with the insulating polymer, e.g., a coupling agent such as avidin or strepavidin and (d) a plurality of chip-associated oligos conjugated to the polymer surface in a specific orientation. In this embodiment, the insulating polymers act as both diluent groups and anchoring groups, with the anchoring group complexes facilitate the conjugation of the chip-associated oligos by specific distribution of the polymers conjugated to a binding molecule.

In another specific embodiment, the biosensor used for detection of the target agent comprises: (a) a conductive surface, e.g., gold, platinum, or carbon, that functions as an electrode; (b) an electroconductive polymer that is uniformly distributed on the electrode; (c) an adapter molecule associated with the electroconductive polymer, e.g., a coupling agent such as avidin or strepavidin; and (d) a plurality of chip-associated oligos conjugated to the polymer surface in a specific orientation. The electroconductive polymer coating on the electrode performs a dual function, serving both to bind the chip-associated oligo to the surface of the electrode, and to render the electrode sensitive to variations in the composition of the buffer solution. In particular, changes in the composition of the buffer solution which affect the redox composition of the electroconductive polymer result in a corresponding change in the steady state potential of the detection electrode. The electroconductive polymers facilitate the conjugation of the chip-associated oligos for detection of the target agent in a sample by specific distribution of polymers conjugated to a binding molecule, e.g., avidin or strepavidin. The avidin or strepavaidin in return provides a blocking agent to prevent reducing non-specific interactions of the sample with the conductive surface due to the blocking of the free surfaces of the diluent groups (i.e., the electroconductive polymer) by the avidin or strepavidin.

Preferably, the electroconductive polymer used on the chip is an ionically conductive biocompatible polymer, which is capable of reversible reduction-oxidation: Such biocompatible polymers can be insulating materials that become electrically conductive in the presence of fluids with specific ions present. Examples of such polymers include, but are not limited to, polytetrafluoroethylene (PTFE). The conditions under which such polymers are used will be determinative of their insulating or conductive behavior. In a specific aspect of this embodiment, the electroconductive polymer can be doped with dopant anions, e.g., dodecyl sulphate or dextran sulphate.

Adaptor molecules may either be immobilized in the electroconductive polymer film at the electrochemical synthesis stage by adding adaptor molecules to the electrochemical polymerization solution or may be adsorbed onto the surface of the electroconductive polymer film after electrochemical polymerization. In the former case, a solution of adaptor molecules may be added to the electrodeposition solution immediately before the deposition process. The deposition process works optimally if the storage time of the finished solution does not exceed 30 minutes. Depending on the particular type of test, the concentration of adaptor molecules in the solution may be varied in the range 5.00-100.00 μ/ml. Procedures for electrodeposition of the electroconductive polymer from the solution containing adaptor molecules are described in the examples included herein. On completion of electrodeposition process, the detection electrode obtained may be rinsed successively with deionized water and 0.01 M phosphate-saline buffer solution and, depending on the type of test, may then be placed in a special storage buffer solution containing microbial growth inhibitors or bactericidal agents (e.g., gentamicin), or dried in dust-free air at room temperature.

Where the adaptor molecules are to be adsorbed after completion of the electrodeposition process the following protocol may be used (although it is hereby stated that the invention is in no way limited to the use of this particular method), the detection electrode is first rinsed with deionized water and placed in freshly prepared 0.02M carbonate buffer solution, where it is held for 15-60 minutes. The detection electrode is then placed in contact with freshly-prepared 0.02M carbonate buffer solution containing adaptor molecules at a concentration of 1.00-50.00 μg/ml, by immersing the detection electrode in a vessel filled with solution, or by placing a drop of the solution onto the surface of the detection electrode. The detection electrode is incubated with the solution of adaptor molecules, typically for 1-24 hours at +4° C. After incubation, the detection electrode is rinsed with deionized water and placed for 1-4 hours in a 0.1M phosphate-saline buffer solution. Depending on the type of test, the detection electrode may then be placed either in a special storage buffer solution containing microbial growth inhibitors or bactericidal agents, or dried in dust-free air at room temperature.

When the adaptor molecules are avidin or strepavidin, the above-described methods of the invention for comprise a further step of contacting the coated electrode with a solution comprising specific oligos conjugated with biotin such that said biotinylated oligos bind to molecules of avidin or strepavidin immobilized in or adsorbed to the electroconductive polymer coating of the electrode via a biotin/avidin. or biotin/strepavidin binding interaction. Conjugation of biotin with the corresponding oligo, a process known to those skilled in the art as biotinylation, can be carried out using procedures well known in the art.

Biotinylated peptidic spacers, generally from between 0.4 and 2 nm in length, can also be used to couple the adaptor molecule to the oligo. The resulting conjugates can be immobilized on the microdevice electrode surface through specific binding to the adaptor molecule. The electron transfer through multilayers of the conjugates is strongly dependent on the length of the spacer between the oligo (and thus any bound electrochemical detection agent) and the electrode surface. The redox current through the layer is dependent on external parameters such as the applied voltage difference between the two electrode arrays or the temperature.

In one embodiment, the electrostatic interactions between a detection moiety and the SAM can be controlled though the use of immobilized adsorbates on the monolayer and control of the pH in the reaction solution. This will allow enhancement of the electron signaling through better control of the distance between the detection moiety and the electrode monolayer. In one example, the detection moiety is negatively charged, and the monolayer is modified with a deprotonable adsorbate. For example, in the case where the immobilized adsorbate is a carboxylic acid, the deprotonation of the carboxylic acid head leads to repulsion of a negatively charged redox molecule (e.g., Fe(CN)₆^{3-/4-}), leading in turn to a decrease in heterogeneous electron transfer. The reaction can thus be enhanced by decreasing the pH of the reaction mixture, allowing the redox reaction to penetrate to the electrode.

In another related embodiment, the detection moiety is positively charged, and the monolayer is modified with an immobilized adsorbate that responds reversibly to pH. For example, the immobilized adsorbates are amine containing adsorbates in combination with a positively charged redox couple (e.g., Ru(NH₃)₆^{2+/3+}). At low pH, when the amines on the dendrimer are protonated, the layer is isolating; at high pH the amines are deprotonated and the redox couple penetrates the dendrimer through which it can reach the electrode.

Other electrochemically active monolayers that combine the reduction of the immobilized adsorbate with protonation include azobenzenes (Caldwell W R et al., J. Am. Chem. Soc. 117:6071 (1995); Wang R et al., J. Electroanal. Chem. 438:213 (1997)), nitrobenzoic acids (Casero E et al., 1999 15:127 (1999)), and mixed acid-ferrocene sulfide molecules (Beulen W J et al., 503 Chem Commun (1999)).

To detect multiple agents in a sample simultaneously, multiple electrodes, or an electrode with multiple chip-associated oligos attached in a predetermined configuration, can be employed. For example, nucleic acid detection sensors, which use an electrochemical technique, can comprise an oligo array or other structural arrangement to detect the multiple agents. In such a configuration, a plurality of electrodes each having a distinct chip-associated oligo molecule affixed thereto or otherwise associated therewith are arranged in predetermined configuration. In a preferred embodiment, the voltage applied to each electrode is equal. Additionally, to verify the hybridization to a particular chip-associated oligo, the device comprising the electrodes preferably includes a switch circuit, a decoder circuit, and/or, a timing circuit to apply the voltage to the individual electrodes and to receive the output signal from each of the electrodes.

Accordingly, in a preferred embodiment, the present invention provides universal oligo chips that comprise substrates comprising a plurality of electrodes, preferably gold, platinum, palladium or semiconductor electrodes. In addition, each electrode is capable of interconnection that is attached to the electrode at one end and is ultimately attached to a device that can control the electrode and/or receive the signal transmitted via conductive means in contact with the electrode. That is, each electrode is independently addressable. The substrates can be part of a larger device comprising a detection chamber that exposes a given volume of sample to the detection electrode. Generally, the detection chamber ranges from about 1 pl to 1 ml, with about 10 μl to 500 μl being preferred. As will be appreciated by those in the art, depending on the experimental conditions and assay, smaller or larger volumes may be used. The volumes and concentrations employed are typically empirically determines using methods readily known to those of ordinary skill in the art.

In certain embodiments, the biosensor comprises a detection chamber and electrode that are part of a cartridge that can be placed into a device comprising electronic components selected from the group comprising potentiometers, AC/DC voltage source, ammeters, processors, displays, temperature controllers, light sources, and the like. In a typical embodiment, the interconnections from each electrode are positioned such that upon insertion of the cartridge into the device, connections between the electrodes and the electronic components are established. The device can also comprise a means for controlling the temperature, such as a peltier block, that facilitates the conditions employed in the hybridization reaction.

In certain preferred embodiments, the electrode is first coated with a biocompatible substance (such as dextran, carboxylmethyldextran, other hydrogels, polypeptides, polynucleotides, biocompatible and/or bio-inert matrices or the like). The chip-associated universal oligo is immobilized to such biocompatible substance.

The chip-associated universal oligos may be immobilized onto the electrodes directly or indirectly by covalent bonding, ionic bonding and physical adsorption. Examples of immobilization by covalent bonding include a method in which the surface of the electrode is activated and the nucleic acid molecule is then immobilized directly to the electrode or indirectly through a cross linking agent. Yet another method using covalent bonding to immobilize a chip-associated universal oligo includes introducing an active functional group into an oligo followed by direct or indirect immobilization. The activation of the surface may be conducted by electrolytic oxidation in the presence of an oxidizing agent, or by air oxidation or reagent oxidation, as well as by covering with a film. Useful cross-linking agents include, but are not limited to, silane couplers such as cyanogen bromide and gamma-aminopropyl triethoxy silane, carbodiimide and thionyl chloride and the like. Useful functional groups to be introduced to the oligo may be, but are not limited to, sulfide, disulfide, amino, amide, amido, a carboxyl, a hydroxyl, carbonyl, oxide, phosphate, sulfate, aldehyde, keto, ester and mercapto groups. Other highly reactive functional groups may also be employed using methods readily known to those of ordinary skill in the art.

To facilitate the screening and/or detection of multiple bioterrorism target agents in a sample, multiple electrodes, or an electrode with multiple chip-associated universal oligos attached, preferably in a predetermined configuration are employed. In such a configuration, a plurality of electrodes each having a distinct chip-associated universal oligo affixed thereto or otherwise associated therewith are arranged in predetermined configuration. In a preferred embodiment, the voltage applied to each electrode is equal. Additionally, to verify the hybridization of a particular chip-associated universal oligo, the electrochemical detection device preferably includes a switch circuit, a decoder circuit, and/or, a timing circuit to apply the voltage to the individual electrodes and to receive the output signal from each of the electrodes. In certain preferred embodiments, the signals are distinguished via the character of the signal, irrespective of configuration.

### Traditional Optical Detection Methods

Alternatively, the capture-associated universal oligos and chip-associated universal oligos can be used in traditional optical detection methods well known in the art. In this case, the chip-associated universal oligos may be synthesized in situ (see, e.g., U.S. Pat. No. 5,744,305; U.S. Pat. No. 5,753,788; U.S. Pat. No. 5,770,456; U.S. Pat. No. 5,889,165; U.S. Pat. No. 6,346,413; U.S. Pat. No. 6,506,558; U.S. Pat. No. 6,566,495; and U.S. Pat. No. 6,600,031) or by physical spotting of the chip-associated universal oligos with the aid of robotic arraying equipment or through electronic addressing on a solid substrate such as glass. The matrix or material that serves as a substrate “chip” on which chip-associated universal oligos are arrayed may be of any type of solid support, and the association may be covalent or noncovalent. The solid support can take on a variety of shapes and compositions, including microparticles, beads, porous and impermeable strips and membranes, the interior surface of reaction vessels such as test tubes and microtiter plates, and the like. Methods for attaching a desired reaction partner to a selected solid support will be a matter of routine skill to one skilled in the art.

For example, covalent immobilization of nucleic acids on a support may be used, and a wide variety of support materials and coupling techniques can be employed. For example, the nucleic acids can be coupled to phosphocellulose through phosphate groups activated by carbodiimide or carbonyldiimidazole (see, e.g., Bautz, E. K. F., and Hall, B. D., Proc. Nat'l. Acad. Sci. USA 48:400-408 (1962); and Shih, T. Y., and Martin, M. A., Biochem. 13:3411-3418 (1974)). Also, diazo groups on m-diazobenzoyloxymethyl cellulose can react with guanine and thymidine residues of the polynucleotide (see, e.g., Noyes, B. E., and Stark, G. R., 5:301-310; and Reiser, J., et al, Biochem. Biophys. Res. Commun. 85:1104-1112 (1978)). Polysaccharide supports can also be used with coupling through phosphodiester links formed between the terminal phosphate of the polynucleotide and the support hydroxyls by water soluble carbodiimide activation (see, e.g., Richwood, D., Biochem. Biophys. Acta 269:47-50 (1972); and Gilham, P. T., Biochem. 7:2809-2813 (1968)), or by coupling nucleophilic sites on the polynucleotide with a cyanogen bromide activated support (see, e.g., Arndt-Jovin, D. J., et al, Eur. J. Biochem. 54:411-418 (1975); and Linberg, U., and Eriksson, S., Eur. J. Biochem. 18:474-479 (1971)). Further, the 3′-hydroxyl terminus of the nucleic acid can be oxidized by periodate and coupled by Schiff base formation with supports bearing amine or hydrazide groups (see, e.g., Gilham, P. T., Method. Enzymol. 21:191-197 (1971); and Hansske, H. D., et al, Method. Enzymol. 59:172-181 (1979)). Supports having nucleophilic sites can be reacted with cyanuric chloride and then with the polynucleotide (see, e.g., Hunger, H. D., et al, Biochem. Biophys. Acta 653:344-349 (1981)).

In general, any method can be employed for immobilizing the nucleic acid, provided that the chip-associated universal oligo sequence is available for hybridization to the capture-associated universal oligo or polymerization products. Particular methods or materials are not critical to the present invention.

A particularly attractive alternative to employing directly immobilized nucleic acid is to use an immobilizable form of nucleic acid which allows hybridization to proceed in solution where the kinetics are more rapid. Normally in such embodiment, one would use a chip-associated universal oligo which comprises a reactive site capable of forming a stable covalent or noncovalent bond with a reaction partner and obtain immobilization by exposure to an immobilized form of such reaction partner. Preferably, such reactive site in the chip-associated universal oligo is a binding site such as a biotin or hapten moiety which is capable of specific noncovalent binding with a binding substance such as avidin or an antibody which serves as the reaction partner.

Essentially any pair of substances can comprise the reactive site/reactive partner pair which exhibit an appropriate affinity for interacting to form a stable bond, that is a linking or coupling between the two which remains substantially intact during the subsequent assay steps, principally the separation and detection steps. The bond formed may be a covalent bond or a noncovalent interaction, the latter being preferred especially when characterized by a degree of selectivity or specificity. In the case of such preferred bond formation, the reactive site on the chip-associated universal oligo will be referred to as a binding site and the reaction partner as a binding substance with which it forms a noncovalent, commonly specific, bond or linkage.

In such a preferred embodiment, the binding site can be present in a single stranded hybridizable portion or in a single or double stranded nonhybridizable portion of the chip-associated universal oligo or can be present as a result of a chemical modification of the chip-associated universal oligo. Examples of binding sites existing in the nucleotide sequence are where the nucleic acid comprises a promoter sequence (e.g., lac-promoter, trp-promoter) which is bindable by a promoter protein (e.g., bacteriophage promoters, RNA polymerase), or comprises an operator sequence (e.g., lac operator) which is bindable by a repressor protein (e.g., lac repressor), or comprises rare, antigenic nucleotides or sequences (e.g., 5-bromo or 5-iododeoxyuridine, Z-DNA) which are bindable by specific antibodies (see also British Pat. No. 2,125,964). Binding sites introduced by chemical modification of the polynucleotide comprised in the chip-associated universal oligo are particularly useful and normally involve linking one member of a specific binding pair to the chip-associated universal oligo. Useful binding pairs from which to choose include biotin/avidin (including, for example, egg white avidin and strepavidin), haptens and antigens/antibodies, carbohydrates/lectins, enzymes/inhibitors, and the like. Where the binding pair consists of a proteinaceous member and a nonproteinaceous member, it will normally be preferred to link the nonproteinaceous member to the chip-associated universal oligo since the proteinaceous member may be unstable under the denaturing conditions of hybridization of the chip-associated universal oligo to the capture-associated universal oligo or to the polymerization products. Preferable systems involve linking the chip-associated universal oligo with biotin or a hapten and employing immobilized avidin or anti-hapten antibody reagent, respectively.

Labels are attached to the capture-associated universal oligos (or the capture moiety) and detected with an array reader that quantitates the level of optical activity (typically, fluorescence) and identifies the location of the hybridization event. Typically the reader involves confocal optical detection as discussed in detail infra. In the present embodiment, the label is added directly to the capture-associated universal oligo or to capture moiety (e.g., the antibody). Means of attaching labels to nucleic acids are well known to those of skill in the art and include, e.g., end-labeling by kinasing the universal oligo and subsequent attachment (e.g., ligation) of a nucleic acid linker joining the sample nucleic acid to a label (e.g., a fluorophore). Useful labels this embodiment of the present invention include fluorescent dyes (e.g., fluorescein, texas red, rhodamine, green fluorescent protein, and the like. Patents teaching the use of labels include, inter alia, U.S. Pat. Nos. 3,817,837; 3,850,752; 3,939,350; 3,996,345; 4,277,437; 4,275,149; and 4,366,241.

If amplification is performed as described in detail infra, the capture-associated universal oligo and the chip-associated universal oligo will have the same or substantially the same sequence and the amplification products will be complementary (or substantially complementary) to the capture-associated universal oligos and the chip-associated universal oligos.

### Assay and Detection

The universal oligos and universal oligo chips are used in a system comprising capture-associated universal oligos, where the capture moiety is one or more moieties specific for bioterrorism target agents such as those listed in Tables 1a and 1b. The capture-associated universal oligos are contacted/mixed with a sample that is suspected of containing the bioterrorism target agents, under conditions that if a bioterrorism target agent is present, the capture moiety can react with, i.e., bind or otherwise associate with/to the bioterrorism target agent. In most embodiments, the capture-associated universal oligos conjugated to the capture moieties are added in excess relative to the amount of bioterrorism target agent suspected to be present in the sample.

If multiple capture-associated universal oligos are used, each having a capture moiety specific for a different target agent or different portion of the same target agent, multiple immobilized binding partners are used to facilitate the removal/separation of unreacted capture-associated universal oligos (those with capture moieties that did not react with target agent in the sample). In such a detection method, multiple different target agents may be screened/detected simultaneously. The advantage of a simultaneous accurate detection method includes an increased speed at which multiple suspected target agents can be eliminated.

With these concepts in mind, in one application of one embodiment of the invention, capture-associated universal oligos are conjugated to capture moieties capable of binding to or otherwise associating with bioterrorism target agents. In accordance with this embodiment the invention the following elements are included: (1) a chip-associated universal oligo immobilized on a surface, (2) a capture-associated universal oligo that is complementary to the chip-associated universal oligo, where the capture-associated universal oligo is conjugated to an capture moiety corresponding to one or more bioterrorism target agents, (3) immobilized binding partners, and (4) a sample suspected of containing the one or more bioterrorism target agents. In one aspect, the capture-associated universal oligo is contacted with the sample to form a first mixture, then the first mixture is contacted with the immobilized binding partners. The unreacted capture-associated universal oligos are captured by the immobilized binding partners, thereby removing the unreacted capture-associated universal oligos from solution. The solution phase of the mixture is then contacted with the chip-associated universal oligos, followed by detection as otherwise described herein. Alternatively, the reacted oligo-capture moiety-target agent moieties can be immobilized with an immobilized binding partner to the capture moiety/target agent complex, leaving the unreacted oligo-capture moiety molecules in solution. Other variations on this preferred embodiment include one or more other aspects of the invention described herein or such other modification known to those of ordinary skill in the art.

This embodiment most frequently is employed in a multi-target (so-called multiplexed) format, allowing for the screening of multiple bioterrorism target agents simultaneously. Such embodiments include providing (1) a detection device comprising chip-associated universal oligos, (2) a set of capture-associated universal oligos, (3) a sample suspected of containing bioterrorism target agents, and (4) immobilized binding partners to the capture moieties of the capture-associated universal oligos. The method comprises mixing/contacting the sample with the capture-associated universal oligos under reaction conditions that allow the capture moieties to capture bioterrorism target agents present in the sample to form a first mixture. The first mixture is then mixed/contacted with the immobilized binding partners to the capture moieties where the capture moieties that have not reacted with bioterrorism target agents in the sample react with the immobilized binding partners to form an immobilized phase and a solution phase. The solution phase comprises the capture-associated universal oligos that have reacted with bioterrorism target agents in the sample and the immobilized phase comprises the capture-associated universal oligos that did not bind bioterrorism target agents and instead bound the immobilized binding partners. The solution is introduced to the universal oligo chip and the detection device under conditions such that the capture-associated universal oligos present will hybridize to a complementary chip-associated universal oligo, generating a signal.

Alternatively, the reacted capture-associated universal oligo complex can be captured (e.g., by a binding partner that recognizes a different portion of the bioterrorism target agent or the capture moiety/bioterrorism target agent complex) leaving the unreacted capture-associated universal oligos in solution. The immobilized phase is separated, and the reacted capture-associated universal oligo complex is then released into solution and introduced to the universal oligo chip and to the detection device under reaction conditions such that the capture-associated universal oligos and chip-associated universal oligos may hybridize to each other. A signal generated by the hybridization of complementary capture-associated universal oligos and chip-associated universal oligos.

In an alternative embodiment of the invention, capture-associated universal oligos are conjugated to a scaffold which also comprises capture moieties to form a, loaded scaffold, and the target agent of interest is a bioterrorism target agent. In accordance with this embodiment the invention the following elements are included: (1) a chip-associated universal oligo immobilized on a surface, where the surface comprises an electrode, (2) a capture-associated universal oligo that is complementary to the chip-associated universal oligo, where the capture-associated universal oligo is associated with a scaffold which also comprises a capture moiety corresponding to the target agent to form a loaded scaffold, (3) immobilized binding partners, and (4) a sample suspected of containing the target agent. In one aspect, the loaded scaffold is contacted with the sample to form a first mixture, then the first mixture is contacted with the immobilized binding partners. The unreacted loaded scaffolds are captured by the immobilized binding partners and the reacted loaded scaffolds are left in solution, thereby separating the unreacted loaded scaffolds from the reacted loaded scaffolds. The capture-associated universal oligos associated with the reacted loaded scaffolds may then undergo optional amplification via linear or logarithmic methods known in the art. The solution phase of the mixture is then contacted with the chip-associated universal oligos, followed by electrochemical detection as otherwise described herein. Alternatively, the reacted loaded scaffold-target agent moieties can be immobilized with an immobilized binding partner to a different portion of the target agent or to the capture moiety/target agent complex, thereby immobilizing the reacted loaded scaffolds and leaving the unreacted loaded scaffolds in solution.

In yet another embodiment, a reverse bead/scaffold capture method is used where the immobilized binding partner is contacted with the sample to form a first mixture, then this mixture is contacted with the loaded scaffold. The loaded scaffolds bind to a different portion of the target agent or to the immobilized binding partner/target agent complex, and is captured by the immobilized binding partner, leaving the unreacted loaded scaffolds in solution with detection proceeding as described elsewhere herein. Other variations on this preferred embodiment include one or more other aspects of the invention described herein or such other modification known to those of ordinary skill in the art.

This embodiment most frequently is employed in a multi-target (multiplexed) format, allowing for the screening of multiple target antigens simultaneously. Such embodiments include providing (1) an electrochemical detection device comprising chip-associated universal oligos, (2) a set of loaded scaffolds, (3) a sample suspected of containing the target agents, and (4) immobilized binding partners of the capture moieties on the loaded scaffold. The method comprises mixing/contacting the sample with the loaded scaffolds under reaction conditions that allow the capture moieties to capture target agent present in the sample to form a first mixture. The first mixture is then mixed/contacted with the immobilized binding partners to the capture moieties. The capture moieties on the loaded scaffolds that have not reacted with target agents in the sample (unreacted loaded scaffolds) react with the immobilized binding partners to form an immobilized phase. The solution phase comprises the loaded scaffolds that have reacted with target agents (reacted loaded scaffolds) in the sample. The capture-associated universal oligos associated with the reacted loaded scaffolds may then undergo optional amplification via linear or logarithmic methods known in the art. The solution is introduced to the universal oligo chip and the electrochemical detection device under conditions such that the capture-associated universal oligos present will hybridize to a complementary chip-associated universal oligo, generating an electrochemical signal. Alternatively, the reacted loaded scaffold complex can be captured (e.g., by an immobilized binding partner that recognizes a different portion of the target agent or to the capture moiety-target agent complex) leaving the unreacted loaded scaffold complexes in solution. The immobilized phase is separated, and the reacted loaded scaffold complex is then released into solution. The capture-associated universal oligos associated with the reacted loaded scaffolds may then undergo optional amplification via linear or logarithmic methods known in the art. The solution is introduced to the universal oligo chip and to the electrochemical detection device under reaction conditions such that the capture-associated universal oligos and chip-associated universal oligos may hybridize to each other. An electrochemical signal generated by the hybridization of complementary capture-associated universal oligos and chip-associated universal oligos. In various embodiments, the capture-associated universal oligos that are associated with the reacted loaded scaffolds may be subjected to a cleavage reaction and/or a linear or logarithmic amplification step after being separated from unreacted loaded scaffolds but before being contacted with the chip-associated universal oligos.

Yet another alternative embodiment of the multi-target (multiplexed) format, the immobilized binding partner(s) is contacted with the sample, and this first mixture is then contacted with the loaded scaffolds in a reverse bead/scaffold capture scenario. Such embodiment includes providing (1) an electrochemical detection device comprising chip-associated universal oligos, (2) immobilized binding partners corresponding to the target agents, (3) a sample suspected of containing the target agents, and (4) a set of loaded scaffolds where the scaffold comprises a capture-associated universal oligos that is complementary to the chip-associated universal oligo and which also comprises a capture moiety that binds to a different portion of the target agent or to the target agent/binding partner. The method comprises mixing/contacting the sample with the immobilized binding partner under reaction conditions that allow the immobilized binding partners to capture a target agent in the sample to form a first mixture. The first mixture is then mixed/contacted with the loaded scaffolds where the loaded scaffolds bind to a different portion of the target agent that has been captured by the immobilized binding partner or to the immobilized binding partner/target agent complex. The loaded scaffold will bind to the reacted immobilized binding partners, leaving the unbound loaded scaffolds in solution. The immobilized phase is separated, and the reacted loaded scaffold complexes are then released into solution. The capture-associated universal oligos associated with the reacted loaded scaffolds may then undergo optional amplification via linear or logarithmic methods known in the art. The solution is introduced to the universal oligo chip and to the electrochemical detection device under reaction conditions such that the capture-associated universal oligos and chip-associated universal oligos may hybridize to each other. An electrochemical signal is generated by the hybridization of complementary capture-associated universal oligos and chip-associated universal oligos. In various aspects of this embodiment, the capture-associated universal oligos that are associated with the reacted loaded scaffolds may be subjected to a cleavage reaction and/or a linear or logarithmic amplification step after being separated from unreacted loaded scaffolds but before being contacted with the chip-associated universal oligos.

The binding reaction between the bioterrorism target agents in the sample and the capture-associated universal oligos is performed in solution, in a physiological buffer such as phosphate buffered saline (PBS) supplemented with a non-specific blocking agent, such as fetal or new-born calf serum, and may be used when the bioterrorism target agent to be detected is normally found under physiological conditions. However, the methods of the present invention are not limited to detecting bioterrorism target agents only found in physiological conditions. Those of skill in the art would appreciate and understand that different ligands may be used in different conditions without affecting the ability to bind the particular bioterrorism target agent to be detected. The binding reaction can be performed at a temperature within the range of 0° C. to 100° C., preferably at a temperature between 2° C. and 40° C., and more preferably within the range of about 4° C. to about 37° C., and most preferably within the range of about 18° C. to about 25° C. The binding reaction is typically conducted from about 5 minutes to 12 hours, preferably from about 10 minutes to 6 hours, and more preferably from about 15 minutes to 1 hour. The duration of the binding reaction depends on several factors, including the temperature, suspected concentration of the bioterrorism target agent, ionic strength of the sample, and the like. For example, a binding reaction may require 15 minutes incubation at a temperature of 18° C., or 30 minutes incubation at a temperature of 4° C.

Since often bioterrorism target agent antigens and antibodies are involved in the capture reaction as in the binding reaction, typically the capture reaction between the reacted and unreacted capture-associated universal oligos and the immobilized binding partners is performed under conditions much like the binding reaction. The capture reaction also takes place in solution, in a physiological buffer such as phosphate buffered saline (PBS) supplemented with a non-specific blocking agent, such as fetal or new-born calf serum. The capture reaction can be performed at a temperature within the range of 0° C. to 100° C., preferably at a temperature between 2° C. and 40° C., and more preferably within the range of about 4° C. to about 37° C., and most preferably within the range of about 18° C. to about 25° C. The capture reaction is typically conducted from about 5 minutes to 12 hours, preferably from about 10 minutes to 6 hours, and more preferably from about 15 minutes to 1 hour. Those of skill in the art would appreciate and understand the particular the specific time required for the reaction to be performed.

The removal of excess, unreacted capture-associated universal oligos can be achieved by providing immobilized binding partner(s) to the specific capture moiety that is conjugated to the capture-associated universal oligos. The immobilized binding partner is bound to a matrix that is a vessel wall or floor. Alternatively, the matrix may be a column or filter, such as Sepharose 2B, Sepharose 4B, Sepharose 6B, CNBR-Activated Sepharose 4B, AH-Sepharose 4B, CH-Sepharose 4B, Activated CH-Sepharose 4B, Epoxy-Activated Sepharose 6B, Activated Thiol-Sepharose 4B, Sephadex, CM-Sephadex, ECH-Sepharose 4B, EAH-Sepharose 4B, NHS-Activated Sepharose or Thiopropyl Sepharose 6B, etc., all of which are supplied by Pharmacia; Bio-Gel A, Cellex, Cellex AE, Cellex-CM, Cellex PAB, BIO-GEL P, Hydrazide BIO-GEL P, Aminoethyl Bio-Gel P, Bio-Gel CM, Affi-Gel 10, Affi-Gel 15, Affi-Prep 10, Affi-Gel HZ, Affi-Prep HZ, Affi-Gel 102, CM Bio-Gel A, Affi-Gel Heparin, Affi-Gel 501 or Affi-Gel 601, etc., all of which are supplied by Bio-Rad; Chromagel A, Chromagel P, Enzafix P-HZ, Enzafix P-SH OR Enzafix P-AB, etc., all of which are supplied by Wako Pure Chemical Industries Ltd.; AE-Cellurose, CM-Cellurose or PAB Cellurose etc., all of which are supplied by Serva, over which the mixture of reacted and unreacted conjugated nucleic acid molecules can be passed. Similarly, the matrix may include a suspension of particulate matter in a solution, such as microscopic and/or macroscopic beads/particles, where the immobilized binding partner is immobilized on the beads or particle such as polystyrene-, cellulose-, latex-, silica-, polyaminostyrene-, agarose-, polydimethylsiloxane-, or polyvinyl-based beads. In a method using particles, the unreacted nucleic acid molecules will be retained on the semi-solid support created by the particles, whereas the reacted nucleic acid molecules will be eluted through the semi-solid support. Thus, only those capture-associated universal oligos that have bound the particular bioterrorism target agent will be available for hybridization. Alternatively, the particles can include an immobilized binding partner specific for the bioterrorism target agent or for the bioterrorism target agent/capture moiety complex. In this embodiment, only those capture-associated universal oligos conjugated to a capture moiety that has reacted with the bioterrorism target agent in the sample will be retained on the particles or matrix, and the unreacted nucleic acid molecules will pass through. The retained, reacted capture-associated universal oligos then may be selectively released/eluted by known methods including but not limited to the cleavage step, discussed in detail below.

When employing suspensions of particulate matter in a solution, unreacted nucleic acid molecules can be separated from the reacted nucleic acid molecules by techniques such as centrifugation, size exclusion chromatography, filtration and the like. In a method using beads, in particular magnetic beads, the separation step can be achieved by applying a magnetic field to the magnetic beads. In some embodiments, the beads will bind with the unreacted capture moieties, leaving the reacted capture moieties in solution and available for hybridization. In other embodiments, the beads will bind with the reacted capture moieties, leaving the unreacted capture moieties in solution. In addition, either the suspension or bead techniques can employ a particle or bead having a secondary capture moiety specific for the bioterrorism target agent to be detected. In this instance only those capture-associated universal oligos are that have reacted with bioterrorism target agents in the sample will be retained on the beads, and the unreacted capture-associated universal oligos are separated from the suspension by known techniques including, but not limited to, centrifugation, size exclusion chromatography, filtration, magnetism and the like. As discussed above, in this particular embodiment of the invention, the retained, reacted capture-associated universal oligos can be selectively released/eluted by known methods including, but not limited to, the cleavage step, as discussed.

The capture-associated universal oligos preferably are provided to the capture reaction in excess, with the excess (i.e., unreacted) capture-associated universal oligos being removed prior to hybridization. This excess is typically determined relative to the suspected level of bioterrorism target agents present in the sample. This relative excess can be from about 1:1 to 1000000:1, preferably 2:1 to about 10000:1, and more preferably from about 4:1 to 1000:1, and most preferably from 5:1 to 100:1. For example, when the capture moiety is an antibody, typically, an excess of capture moiety can be created by adding 1 μg of the capture-associated universal oligo to a sample suspected of containing up to 1 million bioterrorism target agents to be detected. This ratio gives rise to a molar ratio of typically about 4:1, but can vary dependant upon the molecular mass of the antibody and the bioterrorism target agent to be detected.

In some embodiments of the invention, cleavage of the antibody from the capture-associated universal oligos following separation of reacted and unreacted molecules, but prior to hybridization, is preferable. This situation may arise when the reacted capture-associated universal oligos have been selectively bound to a capture moiety that may interfere with hybridization, or detection, because of the physical size or the presence of local areas of electron density on the surface of the capture moiety. Cleavage can be achieved by, for example, a digestive enzyme, i.e., an enzyme that causes hydrolysis of a bond in a molecule, (e.g., proteolytic enzymes, lipases, phosphatases, phosphodiesterases, esterases, etc.), endonucleases, exonucleases, a restriction endonuclease (e.g., EcoRI), or a flap endonuclease (e.g., FEN-1, RAD2, XPG, etc.). The choice of cleavage method will depend on the nature of the conjugation of the capture moiety to the capture-associated universal oligo, and the moiety to be removed via the cleavage reaction. For example, photocleavage may be employed where a photocleavable phosphoramidite is used in lieu of a restriction site. Those of skill in the art will readily appreciate and understand the circumstances under which one particular method of cleavage would be preferred over another method of cleavage.

For example, a digestive enzyme, such as trypsin, can be used when an antibody is conjugated to the capture-associated universal oligo through some peptide linkage; a restriction endonuclease can be used when there is a specific sequence present in the capture-associated universal oligo, susceptible to the particular restriction endonuclease, between the portion of the capture-associated universal oligo that is complementary to the chip-associated universal oligo molecule and the portion of the capture-associated universal oligo that is conjugated to the capture moiety. In preferred embodiments, restriction sites and restriction endonucleases are chosen that allow cleavage of single stranded nucleic acids. Likewise, a flap endonuclease, such as RAD2, or XPG, could be used when there is a specific structure present in the capture-associated universal oligo, susceptible to the particular flap endonuclease, between the portion of the capture-associated universal oligo that is complementary to the chip-associated universal oligo molecule and the portion of the capture-associated universal oligo that is conjugated to the capture moiety. Those of skill in the art would appreciate and understand the particular types of structure susceptible to flap endonuclease cleavage.

Where it is intended that a restriction endonuclease will be used to separate the antibody from the capture-associated universal oligo, the capture-associated universal oligo will be engineered to contain a specific restriction site between the portion of the capture-associated universal oligo that is complementary to the chip-associated universal oligo molecule and the portion of the capture-associated universal oligo that is conjugated to the antibody. This restriction site will be designed, and the appropriate restriction endonuclease selected, to cleave only in the portion of the capture-associated universal oligo that is conjugated to the antibody and not in the region of complementarity to the chip-associated nucleic acid molecule.

In those embodiments where such cleavage is performed, the cleavage reaction is performed after the capture reaction has been completed and after a selective purification reaction is employed in order to segregate the desired reaction product (i.e., the composition comprising the capture moiety and bioterrorism target agent); for example, the reaction product can be subjected to a secondary capture (e.g., using a secondary immobilized antibody) followed by separation and wash procedures. The immobilized capture-associated universal oligo complex may then be eluted or otherwise separated from its immobilized substrate and the resulting solution containing the released capture-associated universal oligo transferred to chip for hybridization and detection.

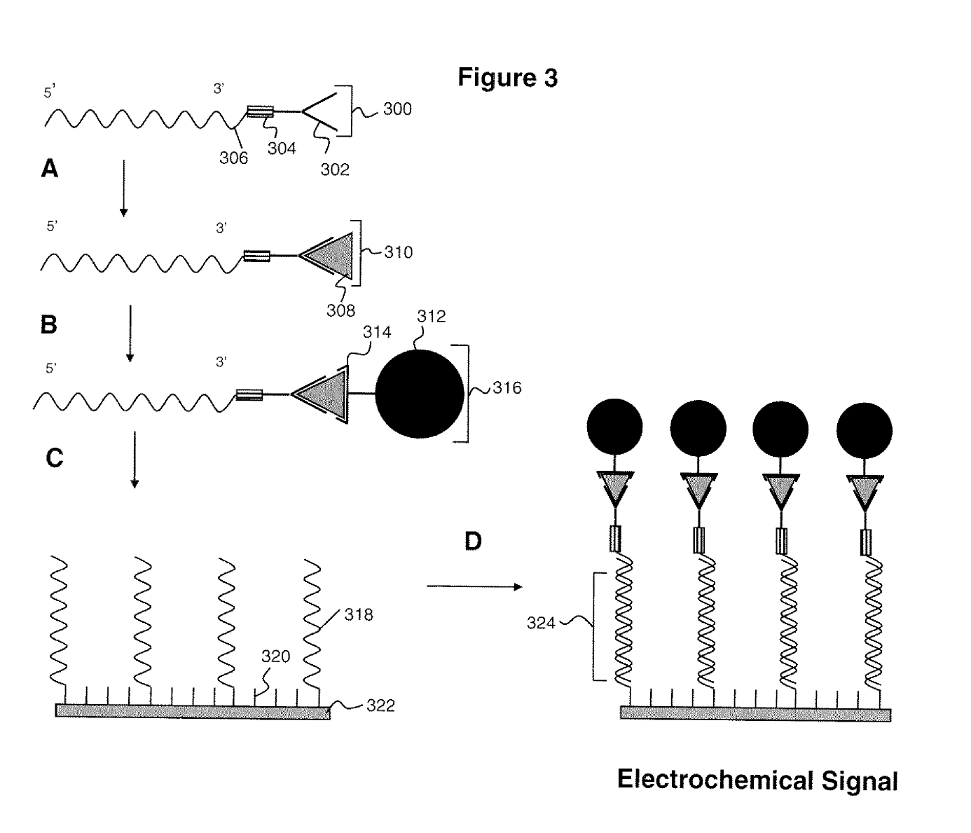

In preferred embodiments, it may be beneficial to use isothermal amplification to increase the number of nucleic acids available for binding to the chip-associated universal oligos, thus enhancing the signal created through complementary binding. As shown in FIG. 3, following binding of the bioterrorism target agent to the capture moiety and isolation from the sample, an oligonucleotide encoding the 5′ to 3′ polymerase recognition sequence is introduced to the capture moiety-bioterrorism target agent complex, and its binding to the complex creates a double-stranded polymerase recognition site (Step A). Following annealing of the oligonucleotide, an excess of single nucleotides and the appropriate polymerase are added to a solution containing the isolated capture moiety-bioterrorism target agent complex, and conditions created to allow for polymerization and linear amplification. Step B comprises (i) exposing the template nucleic acid complex to an aqueous solution comprising the polymerase and an excess of NTP or dNTP and (ii) permitting the polymerase and reactants to create an intermediate duplex comprising a double stranded DNA having a first 5′ end which bears a phage-encoded RNA polymerase recognition site. This reaction continues as the polymerase displaces the double-stranded nucleic acid, resulting in multiple copies of the polymerization products (Step C). In such an embodiment, the chip-associated universal oligo will have the same sequence as the capture-associated oligo, and both will be complementary to the linear polymerization products.

In a preferred embodiment, the polymerase recognition site created by this double stranded region is a phage-encoded RNA polymerase recognition sequence. Exemplary polymerases useful in such isothermal amplification reactions include RNA phage polymerases, including but not limited to T3 polymerase, SP6 polymerase, and T7 polymerase. In one embodiment, a mutant phage-encoded polymerase (e.g., the T7 RNA polymerase mutant Y639F or S641A) is used to allow creation of DNA rather than RNA. This embodiment increases the stability of the synthesized nucleic acids for binding to the electrode, and obviates the problem of RNAse activity. Such mutant polymerases include T7 DNA polymerase, as disclosed in U.S. Pat. No. 6,531,300, U.S. Pat. No. 6,107,037, U.S. Pat. No. 5,849,546, and Padilla and Sousa, Nucleic Acids Res 27(6):1561-1563 (1999).

A number of different nucleotides can be used in the isothermal linear amplification reaction. These include not only the naturally occurring nucleoside mono-, di-, and triphosphates: deoxyadenosine mono-, di- and triphosphate; deoxyguanosine mono-, di- and triphosphate; deoxythymidine mono-, di- and triphosphate; and deoxycytidine mono-, di- and triphosphate (referred to herein as dA, dG, dT and dC or A, G, T and C, respectively). Nucleotides also include, but are not limited to, modified nucleotides and nucleotide analogs such as deazapurine nucleotides, e.g., 7-deaza-deoxyguanosine (7-deaza-dG) and 7-deaza-deoxyadenosine (7-deaza-dA) mono-, di- and triphosphates, deutero-deoxythymidine (deutero-dT) mono-, di- and triphosphates, methylated nucleotides e.g., 5-methyldeoxycytidine triphosphate, 13C/15N labeled nucleotides and deoxyinosine mono-, di- and triphosphate. When using dNTPs and a traditional RNA polymerase, dUTP is substituted for dTTP. For those skilled in the art, it will be clear upon reading the present disclosure that modified nucleotides and nucleotide analogs that utilize a variety of combinations of functionality and attachment positions can be used in the present invention.

Asymmetric amplification using a heat stable polymerase such as Thermus aquaticus polymerase can also be used to create polymerization products complementary to the chip-associated universal oligos. Suitable methods of asymmetric amplification are described in U.S. Pat. No. 5,066,584, which is incorporated by reference in its entirety. When this technique is used, an oligonucleotide complementary to the 3′ end of the capture-associated universal oligo is used under conditions to create a series of polymerization products. In such an embodiment, the chip-associated universal oligo will have the same sequence as the capture-associated universal oligo, and both will be complementary to the asymmetric polymerization products.

Amplification using the Phi29 polymerase may also be used to create polymerization products complementary to the chip-associated universal oligo. Such methods are described in U.S. Pat. No. 5,712,124 and U.S. Pat. No. 5,455,166, both of which are incorporated by reference in their entirety. In brief, the Phi29 polymerase method will allow amplification of the capture-associated universal oligo at a single temperature by utilizing the Phi29 polymerase in conjunction with an endonuclease that will nick the polymerized strand, allowing the polymerase to displace the strand without digestion while generating a newly polymerized strand. As with asymmetric amplification, an oligonucleotide complementary to the 3′ end of the capture-moiety associated nucleic acid is used under conditions to create a series of polymerization products complementary to the capture-associated universal oligo. In such an embodiment, the chip-associated universal oligo will have the same sequence as the capture-associated universal oligo, and both will be complementary to the asymmetric polymerization products.

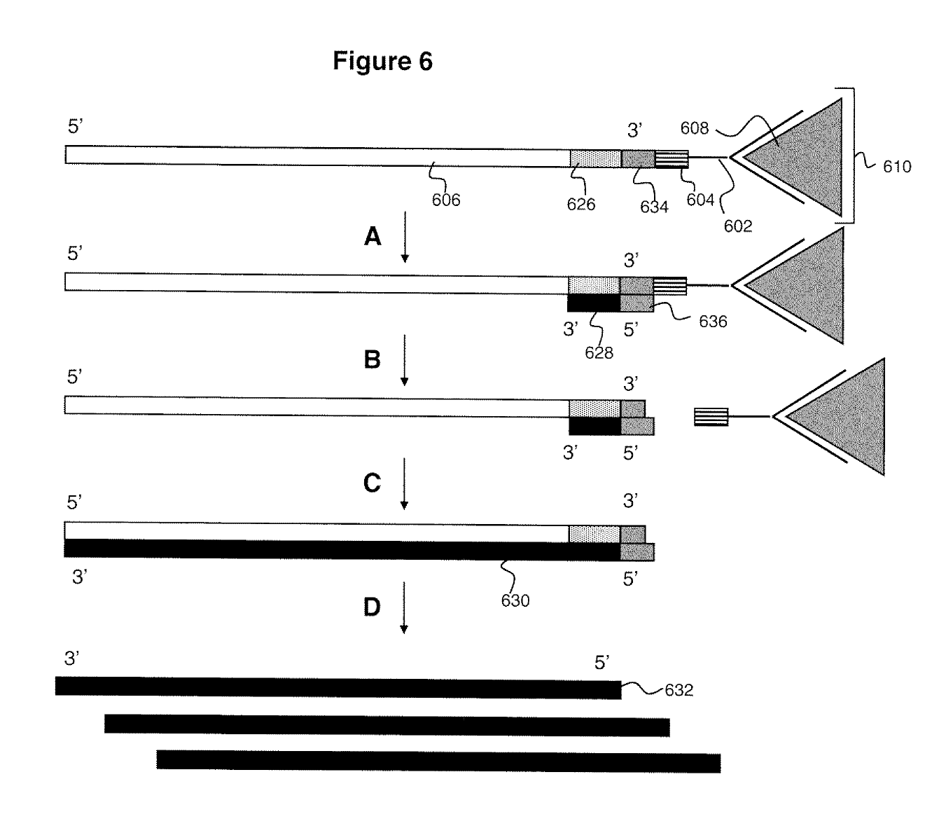

In a particular embodiment of the invention, the capture moiety-bioterrorism target agent complex is cleaved from the capture-associated universal oligo prior to linear or asymmetric amplification. A representative, non-limiting illustration of one such embodiment is illustrated in FIG. 6. Following binding of the bioterrorism target agent to the capture moiety and isolation from the sample, an oligonucleotide encoding the 5′ to 3′ polymerase recognition sequence and a restriction endonuclease sequence is introduced to the capture-associated universal oligo, and its binding to the capture-associated universal oligo creates both a double-stranded polymerase recognition site and a restriction endonuclease cleavage site (Step A). Following annealing of the oligonucleotide to the capture-associated universal oligo, the complex is exposed to the appropriate restriction endonuclease under conditions to allow the cleavage of the capture moiety-bioterrorism target agent from the capture-associated universal oligo (Step B). The restriction endonuclease is then optionally inactivated (e.g., through heat inactivation by exposing the solution to a temperature of 65° C. for 10 minutes), and the capture-associated universal oligo is optionally isolated from the cleaved capture moiety-bioterrorism target agent complex. Following cleavage and optional inactivation or isolation, the capture-associated universal oligo with the bound oligonucleotide is exposed to an aqueous solution comprising an excess of single nucleotides and the appropriate polymerase, under conditions to allow for polymerization and linear amplification. Step C comprises (i) exposing the capture-associated universal oligo complex to an aqueous solution comprising the polymerase and an excess of NTP or dNTP and (ii) permitting the polymerase and reactants to create an intermediate duplex comprising a double stranded DNA having a first 5′ end which bears a phage-encoded RNA polymerase recognition site. This reaction continues as the polymerase displaces the doubles stranded nucleic acid, resulting in multiple copies of the capture-associated, universal oligo (Step D). In such an embodiment, the chip-associated universal oligo will have the same sequence as the capture-associated universal oligo, and both will be complementary to the linear polymerization products.

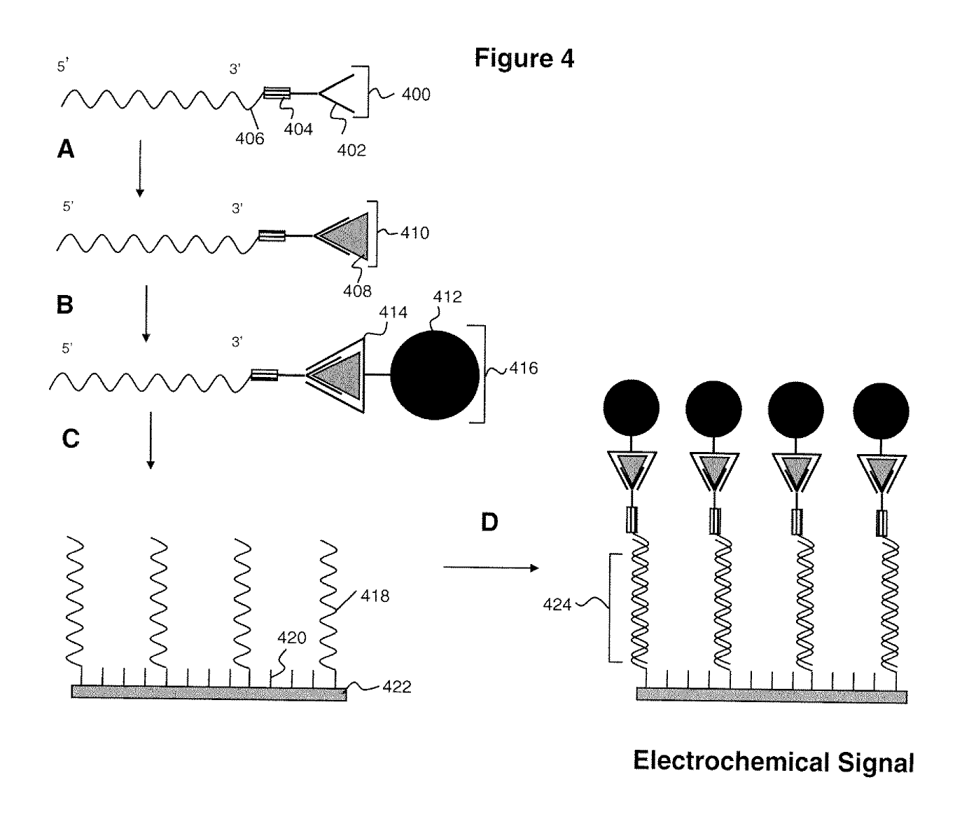

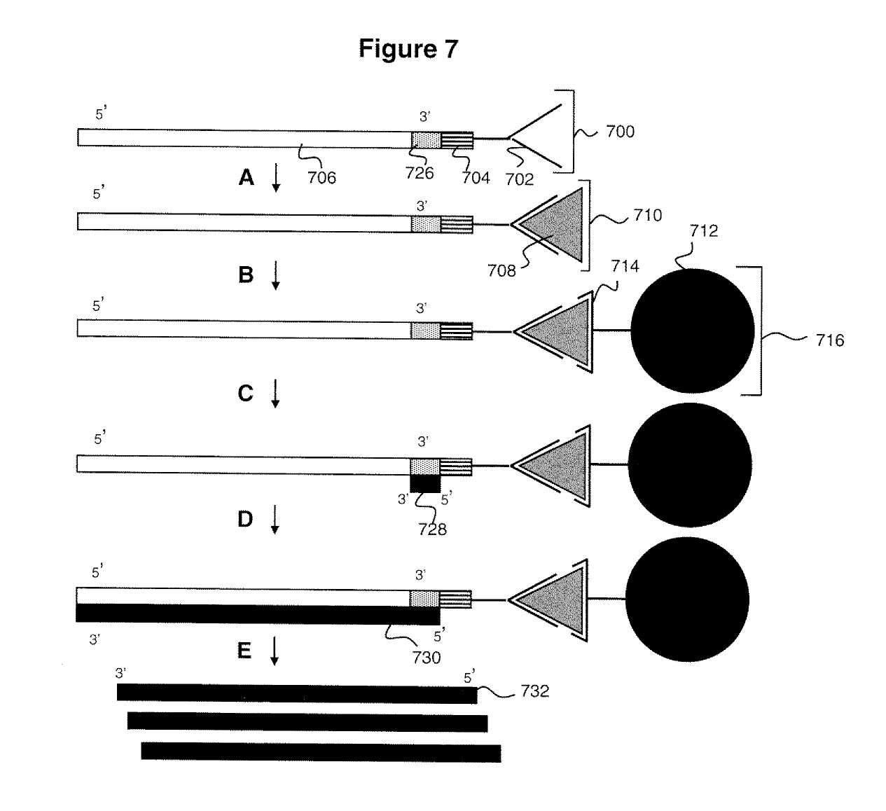

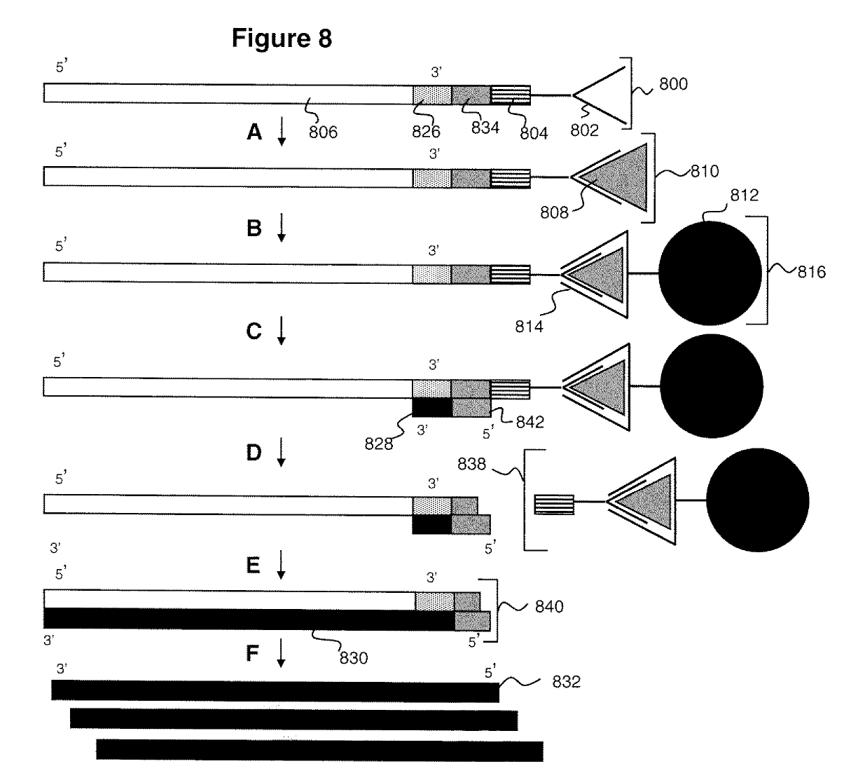

The linear amplification methods using the capture-associated universal oligo can be combined with any of the described isolation methods of the invention, including those described in FIGS. 3 and 4. For example, FIG. 7 illustrates the embodiment of the invention where the capture-associated universal oligo is isolated using an immobilized binding partner which binds to the bioterrorism target agent, and linear amplification using a polymerase recognition site. FIG. 8 illustrates the embodiment of the invention where the capture-associated universal oligo is isolated using an antibody that recognizes an epitope specific to the capture moiety/bioterrorism target agent complex with cleavage of the capture moiety-bioterrorism target agent from the capture-associated universal oligo prior to linear amplification.

The hybridization reaction between the capture-associated universal oligos and the chip-associated universal oligos is typically performed in solution where the metal ion concentration of the buffer is between 0.01 mM to 5 M and a pH range of pH 5 to pH 10. Other components can be added to the buffer to promote hybridization such as dextran sulfate, EDTA, surfactants, etc. The hybridization reaction can be performed at a temperature within the range of 10° C. to 90° C., preferably at a temperature within the range of 25° C. to 60° C., and most preferably at a temperature within the range of 30° C. to 50° C. Alternatively, the temperature is chosen relative to the Tm's of the nucleic acid molecules employed. The reaction is typically performed at an incubation time from 10 seconds to about 12 hours, and preferably an incubation time from 30 seconds to 5 minutes. A variety of hybridization conditions may be used in the present invention, including high, moderate and low stringency conditions; see for example Maniatis et al., Molecular Cloning: A Laboratory Manual, 3rd Edition (2001), hereby incorporated by reference. Persons of ordinary skill in the art will recognize that stringent conditions are sequence-dependent and are dependent upon the totality of the conditions employed. Longer sequences typically hybridize specifically at higher temperatures. Generally, stringent conditions are selected to be about 5-10° C. lower than the thermal melting point (Tm) for the specific sequence at a defined ionic strength pH. Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide. The hybridization conditions may also vary when a non-ionic backbone, i.e., PNA is used, the advantages of using PNA is discussed above. The hybridization reaction can also be controlled electrochemically by applying a potential to the electrodes to speed up the hybridization. Alternatively, the potential can be adjusted to ensure specific hybridization by increasing the stringency of the conditions.

In one embodiment, detection of a hybridization event can be enhanced by the use of a detection moiety. A detection moiety can be, for example, agents characterized by a tendency to bind specifically to double stranded nucleic acid such as double stranded DNA. Many detection moieties have in their molecules a flat intercalating group such as a phenyl group, which intercalates between the base pairs of a double stranded nucleic acid, therefore binding to the double stranded nucleic acid. Some detection moieties comprise conjugated electron structures and are therefore optically active; some are commonly used in the quantification or visualization of nucleic acids. Certain detection moieties exhibit an electrode response, thereby generating or enhancing an electrochemical response. As such, determination of physical change, especially electrochemical change, may serve to detect the intercalating agents bound to a double stranded nucleic acid and so enhance the detection of a hybridization reaction.

Electrochemically active detection moieties useful in the present invention include, but are not limited to, ethidium, ethidium bromide, acridine, aminoacridine, acridine orange, proflavin, ellipticine, actinomycin D, daunomycin, mitomycin C, HOECHST 33342, HOECHST 33258, aclarubicin, DAPI, Adriamycin, pirarubicin, actinomycin, tris(phenanthroline)zinc salt, tris(phenanthroline)ruthenium salt, tris(phenanthroline)cobalt salt, di(phenanthroline)zinc salt, di(phenanthroline)ruthenium salt, di(phenanthroline)cobalt salt, bipyridine platinum salt, terpyridine platinum salt, phenanthroline platinum salt, tris(bipyridyl)zinc salt, tris(bipyridyl)ruthenium salt, tris(bipyridyl)cobalt salt, di(bipyridyl)zinc salt, di(bipyridyl)ruthenium salt, di(bipyridyl)cobalt salt, methylene blue, viologen, anthraquinone, cytochrome C, plastocyanin, cytochrome C′, and the like. Other useful intercalating agents include, inter alia, those listed in Published Japanese Patent Application No. 62-282599. Some of these intercalators contain metal ions and can be considered transition metal complexes. Although the transition metal complexes are not limited to those listed above, complexes which comprise transition metals having oxidation-reduction potentials not lower than or covered by that of nucleic acids are less preferable. The concentration of the intercalator depends on the type of intercalator to be used, but it is typically within the range of 1 ng/ml to 1 mg/ml. Some of these intercalators, specifically Hoechst 33258, has been shown to be a minor-groove binder and specifically binds to double-stranded DNA. The use of such electrochemically active minor groove binders is useful for detection of hybridization in electrochemical detection methods. Thus, in accordance with the present invention, the term “intercalator” is not intended to be limited to those compounds that “intercalate” into the rungs of the DNA ladder structure, but is also intended to include any moiety capable of binding to or with nucleic acids including major and minor groove binding moieties.

Transition metals are those whose atoms have a partial or complete d orbital shell of electrons. Suitable transition metals for use in conjunction with the present invention include, but are not limited to, cadmium (Cd), copper (Cu), cobalt (Co), palladium (Pd), zinc (Zn), iron (Fe), ruthenium (Ru), rhodium (Rh), osmium (Os), rhenium (Re), platinum (Pt), scandium (Sc), titanium (Ti), Vanadium (V), chromium (Cr), manganese (Mn), nickel (Ni), Molybdenum (Mo), technetium (Tc), tungsten (W), and iridium (Ir). That is, the first series of transition metals, the platinum metals (Ru, Rh, Pd, Os, Ir, and Pt), along with Fe, Re, W, Mo and Tc, are preferred. Particularly preferred are ruthenium, rhenium, osmium, platinum, cobalt and iron.

The transition metals can be complexed with a variety of ligands, to form suitable transition metal complexes. As will be appreciated by those in the art, the number and nature of the co-ligands will depend on the coordination number of the metal ion. Mono-, di- or polydentate co-ligands may be used at any position. Suitable ligands fall into two categories: ligands, which use nitrogen, oxygen, sulfur, carbon or phosphorus atoms (depending on the metal ion) as the coordination atoms (generally referred to in the literature as sigma (Σ) donors) and organometallic ligands such as metallocene ligands (generally referred to in the literature as pi (π) donors). Suitable nitrogen donating ligands are well known in the art and include, but are not limited to, NH₂; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol [3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam), EDTA, EGTA and isocyanide. Substituted derivatives, including fused derivatives, may also be used. In some embodiments, porphyrins and substituted derivatives of the porphyrin family may be used. See for example, Comprehensive Coordination Chemistry, Ed. Wilkinson et al., Pergammon Press, 1987, Chapters 13.2 (pp 73-98), 21.1 (pp. 813-898) and 21.3 (pp 915-957), all of which are hereby expressly incorporated by reference.

Suitable sigma donating ligands using carbon, oxygen, sulfur and phosphorus are known in the art. For example, suitable sigma carbon donors are found in Cotton and Wilkinson, Advanced Organic Chemistry, 5th Ed., John Wiley & Sons (1988), hereby incorporated by reference; see, e.g., page 38. Similarly, suitable oxygen ligands include crown ethers, water and others known in the art. Phosphines and substituted phosphines are also suitable; see, e.g., page 38 of Cotton and Wilkinson. The oxygen, sulfur, phosphorus and nitrogen-donating ligands are attached in such a manner as to allow the heteroatoms to serve as coordination atoms.

Such organometallic ligands include cyclic aromatic compounds such as the cyclopentadienide ion [C₅H₅ (−1)] and various ring substituted and ring fused derivatives, such as the indenylide (−1) ion, that yield a class of bis(cyclopentadieyl) metal compounds, (i.e. the metallocenes); see, e.g., Robins et Am. Chem. Soc. 104:1882-1893 (1982); and Gassman et al., J. Am. Chem. Soc. 108:4228-4229 (1986), incorporated by reference. Of these, ferrocene [(C₅H₅)₂Fe] and its derivatives are prototypical examples, which have been used in a wide variety of chemical (Connelly et al., Chem. Rev. 96:877-910 (1996), incorporated by reference) and electrochemical (Geiger et al., Advances in Organometallic Chemistry 23:1-93; and Geiger et al., Advances in Organometallic Chemistry 24:87, incorporated by reference) electron transfer or “redox” reactions. In specific embodiments, the detection moiety is comprised of a plurality of electrochemical detection agents (e.g., ferrocene), optionally linked to a hydrocarbon molecule. Such molecules include but are not limited to ferrocene-hydrocarbon mixtures; such as ferrocene-methane, ferrocene-acetylene, and ferrocene-butane. In one particular embodiment, the detection moiety is Fe(CN)₆^{3-/4-}. (See, Brazill, S. A., Kim, P. H. & Kuhr, W. G., Anal. Chem. 73, 4882-4890 (2001)). Metallocene derivatives of a variety of the first, second and third row transition metals are potential candidates as redox moieties that are covalently attached to the nucleic acid. Other potentially suitable organometallic ligands include cyclic arenes such as benzene, to yield bis(arene)metal compounds and their ring substituted and ring fused derivatives, of which bis(benzene)chromium is a prototypical example. Other acyclic pi-bonded ligands such as the allyl(−1) ion, or butadiene yield potentially suitable organometallic compounds, and all such ligands, in conjunction with other pi-bonded and delta-bonded ligands constitute the general class of organometallic compounds in which there is a metal to carbon bond. Electrochemical studies of various dimers and oligomers of such compounds with bridging organic ligands, and additional non-bridging ligands, as well as with and without metal-metal bonds are potential candidate redox moieties in nucleic acid analysis.

When one or more of the co-ligands is an organometallic ligand, the ligand is generally attached via one of the carbon atoms of the organometallic ligand, although attachment may be via other atoms for heterocyclic ligands. Preferred organometallic ligands include metallocene ligands, including substituted derivatives and the metalloceneophanes (see page 1174 of Cotton and Wilkenson, supra). For example, derivatives of metallocene ligands such as methylcyclopentadienyl, with multiple methyl groups being preferred, such as pentamethylcyclopentadienyl, can be used to increase the stability of the metallocene. In a preferred embodiment, only one of the two metallocene ligands of a metallocene is derivatized.

Alternatively, in some embodiments, the capture-associated universal oligo may be labeled with an electroactive marker. Such electroactive markers can include, but are not limited to, ferrocene derivatives, anthraquinone, silver and silver derivatives, gold and gold derivatives, osmium and osmium derivatives, ruthinium and ruthinium derivatives, cobalt and cobalt derivatives and the like.

When traditional microarray technology is employed using fluorescence to detect a hybridization event between the capture-associated universal oligo and the chip-associated universal oligo, fluorescent detection moieties are utilized. The fluorescent label may be selected from any of a number of different moieties. The preferred moiety is a fluorescent group for which detection is quite sensitive. Various different fluorescence labels techniques are described, for example, in Cambara et al. (1988) “Optimization of Parameters in a DNA Sequenator Using Fluorescence Detection,” Bio/Technol. 6:816 821; Smith et al. (1985) Nucl. Acids Res. 13:2399 2412; and Smith et al. (1986) Nature 321:674 679, each of which is hereby incorporated herein by reference. Fluorescent labels exhibiting particularly high coefficients of destruction may also be useful in destroying nonspecific background signals. In yet other embodiments, the detection moiety is a detection antibody reagent, where the antibody is labeled with a molecular entity which allows detection of nucleic acid binding. Examples of such reagents include, but are not limited to, antibody reagents that preferentially bind to RNA:DNA complexes. Fluorescent detection requires the use of an optical detection device may be used for detection (see, e.g., U.S. Pat. Nos. 5,578,832; 5,631,734; 5,834,758; 6,025,601; 6,141,096 and 6,252,236, the complete disclosures of which are incorporated herein by reference).

Such devices generally employ a scanning device which rapidly sweeps an activation radiation beam or spot across the surface of the chip substrate. Optical detection devices also include focusing optics for focusing the excitation radiation onto the surface of the substrate in a sufficiently small area to provide high resolution of features on the substrate, while simultaneously providing a wide scanning field. An image is obtained by detecting the electromagnetic radiation emitted by the labels on the sample when the labels are illuminated. In some embodiments, fluorescent emissions are gathered by the focusing optics and detected to generate an image of the fluorescence on the substrate surface. The optical detection devices may further employ confocal detection systems to reduce or eliminate unwanted signals from structures above and below the plane of focus of the excitation radiation, as well as auto focus systems to focus both the activation radiation on the substrate surface and the emitted radiation from the surface. Generally, the excitation radiation and response emission have different wavelengths.

In operation, optical detection devices include one or more sources of excitation radiation. Typically, these source(s) are immobilized or stationary point light sources, e.g., lasers such as argon, helium-neon, diode, dye, titanium sapphire, frequency-doubled diode pumped Nd:YAG and krypton. Typically, the excitation source illuminates the sample with an excitation wavelength that is within the visible spectrum, but other wavelengths (i.e., near ultraviolet or near infrared spectrum) may be used depending on the application. In some cases, the label is excited with electromagnetic radiation having a wavelength at or near the absorption maximum of the species of label used. Exciting the label at such a wavelength produces the maximum number of photons emitted. For example, if fluorescein (absorption maximum of 488 nm) is used as a label, an excitation radiation having a wavelength of about 488 nm would induce the strongest emission from the labels.

The excitation radiation from the point source is directed at a movable radiation direction system which rapidly scans the excitation radiation beam back and forth across the surface of the substrate. A variety of devices may be employed to generate the sweeping motion of the excitation radiation. For example, resonant scanner or rotating polygons, may be employed to direct the excitation radiation in this sweeping fashion. Generally, however, galvanometer devices are preferred as the scanning system. In addition, an optical train may be employed between the activation source and the galvanometer mirror to assist in directing, focusing or filtering the radiation directed at and reflected from the galvanometer mirror.

The galvanometers employed in such optical detection devices and systems of the present invention typically sweep a scanning spot across the substrate surface at an oscillating frequency that is typically greater than 30 Hz. The objective lens is preferably selected to provide high resolution, as determined by the focused spot size, while still allowing a wide scanning field.

As the activation radiation spot is swept across the surface of the substrate, it activates fluorescent groups on any capture-associated universal oligos that have bound to the chip-associated universal oligos. The activated groups emit a response radiation or emission which is then collected by the objective lens and directed back through the optical train via the servo mounted mirror. In order to avoid the detrimental effects of reflected excitation radiation upon the detection of the fluorescence, dichroic mirrors or beam splitters may be included in the optical train. These dichroic beam splitters or mirrors are reflective to radiation in the wavelength of the excitation radiation while transmissive to radiation in the wavelength of the response radiation. For example, where an Argon laser is used as the point energy source, it will typically generate activation radiation having a wavelength of about 488 nm. Fluorescence emitted from an activated fluorescein moiety on the other hand will typically have a wavelength between about 515 and 545 nm. As such, dichroic mirrors may be included which transmit light having a wavelength greater than 515 nm while reflecting light of shorter wavelengths. This effectively separates the excitation radiation reflected from the surface of the substrate from the response radiation emitted from the surface of the substrate. Additional dichroic mirrors may be used to separate signals from label groups having different response radiation wavelengths, thereby allowing simultaneous detection of multiple fluorescent indicators.

Following separation of the response radiation from the reflected excitation radiation, the response radiation or fluorescence can be directed at a detector, e.g., a photomultiplier tube, to measure the level of response radiation and record that level as a function of the position on the substrate from which that radiation originated. Typically, the response radiation is focused upon the detector through a spatial filter such as a confocal pinhole. Spatial filters reduce or eliminate unwanted signals from structures above and below the plane of focus of the excitation radiation. Additionally, the device may incorporate a bandpass filter between the dichroic mirror and the detector to further restrict the wavelength of radiation that is delivered to the detector.

In certain preferred embodiments, the polymerization product comprises RNA sequences (e.g., a T7 transcription product) that are complementary to the chip-associated universal oligo. Thus, hybrids resulting from hybridization between the chip-associated universal oligo and the polymerization products will be DNA:RNA duplexes (when the chip-associated universal oligos are DNA) or RNA:RNA duplexes (when the chip-associated universal oligos are RNA). The resulting hybrids can be detected by an antibody reagent capable of binding to the DNA:RNA or RNA:RNA duplexes. A variety of protocols and reagent combinations can be employed in order to carry out this embodiment of the present method.

Detection of a DNA:RNA or RNA:RNA hybrid-recognizing antibody reagent can be accomplished in any convenient manner. In a preferred embodiment, the antibody reagent can be labeled with a moiety such as an enzymatically active group, a fluorescer, a chromophore, a luminescer, a specifically bindable ligand, an electrochemically detectable molecule/moiety, a radioisotope or the like, with the nonradioisotopic labels being especially preferred. The labeled antibody reagent which becomes bound to resulting immobilized hybrid duplexes can be readily separated from that which does not become so bound.

It should be understood that by the expressions “RNA,” “DNA,” “RNA nucleotide sequence,” “DNA nucleotide sequence,” or similar designations herein, it is not implied or intended that all nucleotides comprised in the nucleic acids be ribonucleotides or 2′-deoxyribonucleotides. The fundamental feature of an RNA or DNA capture-associated universal oligo or chip-associated universal oligo for purposes of the present invention is that it be of such character to be detected by antibodies to DNA:RNA hybrids, where individual single strands are not bound by such an antibody and DNA:DNA hybrids are not bound by such an antibody. One or more of the 2′-positions on the nucleotides comprised in the nucleic acids can be chemically modified, provided the antibody binding characteristics necessary for performance of the present assay are maintained to a substantial degree. Likewise, in addition or alternatively to such limited 2′-deoxy modification, a chip-associated universal oligo, capture-associated universal oligo or polymerization product can have in general any other modification along its ribose phosphate backbone provided there is no substantial interference with the specificity of the antibody to the DNA:RNA or RNA:RNA hybridization product compared to individual single strands or to DNA:DNA hybrids.

Where such modifications exist in a nucleic acid, the immunogen used to raise the antibody reagent would preferably comprise one strand having substantially corresponding modifications and the other strand being substantially unmodified RNA or DNA, depending on whether sample RNA or DNA was intended to be detected. Preferably, the modified strand in the immunogen would be identical to the modified strand in an RNA or DNA oligo. An example of an immunogen is the hybrid poly(2′-0-methyladenylic acid):poly(2′-deoxythymidylic acid). Another would be poly(2′-O-ethylinosinic acid):poly(ribocytidylic acid). The following are further examples of modified nucleotides which could be comprised in a modified nucleic acid: 2′-O-methylribonucleotide, 2′-O-ethylribonucleotide, 2′-azidodeoxyribonucleotide, 2′-chlorodeoxyribonucleotide, 2′-O-acetylribonucleotide, and the phosphorothiolates or methylphosphonates of ribonucleotides or deoxyribonucleotides. Modified nucleotides can appear in nucleic acids as a result of introduction during enzymatic synthesis of the nucleic acid from a template. For example, adenosine 5′-O-(1-thiotriphosphate) (ATPαS) and dATPαS are substrates for DNA dependent RNA polymerases and DNA polymerases, respectively. Alternatively, the chemical modification can be introduced after the nucleic acid has been prepared. For example, an RNA oligo can be 2′-O-acetylated with acetic anhydride under mild conditions in an aqueous solvent (see, e.g., Steward, D. L. et al, Biochem. Biophys. Acta 262:227 (1972)).

A detection antibody reagent of certain preferred embodiments of the invention is typically characterized by its ability to bind the DNA:RNA hybrids formed to the significant exclusion of single stranded polynucleotides or to DNA:DNA hybrids. The detection antibody reagent can consist of whole antibodies, antibody fragments, polyfunctional antibody aggregates, or in general any substance comprising one or more specific binding sites from an antibody for DNA:RNA. When in the form of whole antibody, it can belong to any of the classes and subclasses of known immunoglobulins, e.g., IgG, IgM, and so forth. Any fragment of any such antibody which retains specific binding affinity for the hybridized nucleic acid can also be employed; for instance, the fragments of IgG conventionally known as Fab, F(ab′), and F(ab′)₂. In addition, aggregates, polymers, derivatives and conjugates of immunoglobulins or their fragments can be used where appropriate.

The immunoglobulin source for the antibody reagent can be obtained in any available manner such as conventional antiserum and monoclonal techniques. Antiserum can be obtained by well-established techniques involving immunization of an animal, such as a mouse, rabbit, guinea pig or goat, with an appropriate immunogen. The immunoglobulins can also be obtained by somatic cell hybridization techniques, such resulting in what are commonly referred to as monoclonal antibodies, also involving the use of an appropriate immunogen.

Immunogens for stimulating antibodies specific for DNA:RNA hybrids can comprise homopolymeric or heteropolymeric polynucleotide duplexes. Among the possible homopolymer duplexes, particularly preferred is poly(rA).poly(dT) (see, e.g., Kitagawa and Stollar Mol. Immunol. 19:413 (1982)). However, in general, heteropolymer duplexes will be preferably used and can be prepared in a variety of ways, including transcription of pX174 virion DNA with RNA polymerase (see, e.g., Nakazato, Biochem. 19:2835 (1980)). The selected RNA:DNA duplexes are typically adsorbed to a methylated protein, or otherwise linked to a conventional immunogenic carrier material, such as bovine serum albumin, and injected into the desired host animal (see e.g, U.S. Pat. No. 5,200,313, and Stollar, Meth. Enzymol., 70:70 (1980)).

In certain alternative embodiments, it may be preferred to use chip-associated universal oligos that comprise RNA and, as such, RNA:RNA duplexes will be formed during the hybridization process. Antibodies to RNA:RNA duplexes can be substituted for the antibodies to DNA:RNA duplexes described herein without deviating from the present invention. Antibodies to RNA:RNA duplexes can, for example without limitation, be raised against double stranded RNAs from viruses such as reovirus or Fiji disease virus which infects sugar cane, among others. Also, homopolymer duplexes such as poly(rI).poly(rC) or poly(rA) poly(rU), among others, can be employed as above.

In certain preferred embodiments, the “chip associated oligo” will not be immobilized but instead is contacted with the complexed conjugated oligo in solution. The double stranded molecule resulting from a hybridization event, can be captured (i.e., immobilized) by a variety of methods including, inter alia, by immobilized capture antibodies. The methods for such capture are similar to the forgoing examples regarding antibody labeling of the immobilized complex.

The binding of the detection antibody reagent to the hybridized duplex according to the present method can be detected by any convenient technique. Advantageously, the antibody reagent will itself be labeled with a detectable chemical group. Such detectable chemical group can be any material having a detectable physical or chemical property. Such materials have been well-developed in the field of immunoassays and in general most any label useful in such methods can be applied to the present invention. Particularly useful are, inter alia: enzymatically active groups, such as enzymes (see, e.g., Clin. Chem., 22:1243 (1976), U.S. Pat. No. 31,006 and UK Pat. 2,019,408), enzyme substrates (see, e.g., U.S. Pat. No. 4,492,751, cofactors (see, e.g., U.S. Pat. Nos. 4,230,797 and 4,238,565), and enzyme inhibitors (see, e.g., U.S. Pat. No. 4,134,792); fluorescers (see, e.g., Clin. Chem., 25:353 (1979)); chromophores; luminescers such as chemiluminescers and bioluminescers (see, e.g., U.S. Pat. No. 4,380,580); specifically bindable ligands such as biotin (see, e.g., European Pat. Spec. 63,879) or a hapten (see, e.g., PCT Publ. 83-2286); electrochemically detectable reagents/moieties (see, e.g., U.S. Pat. Nos. 5,776,672; 5,972,692 and U.S. Pat. Pub. 2002/01467162004/0063126) and radioisotopes such as ³H, ³⁵S, ³²P, ¹²⁵I, and ¹⁴C. Such labels and labeling pairs are detected on the basis of their own physical properties (e.g., fluorescers, chromophores and radioisotopes) or their reactive or binding properties (e.g., enzymes, substrates, cofactors and inhibitors). For example, a cofactor-labeled antibody can be detected by adding the enzyme for which the label is a cofactor and a substrate for the enzyme. A hapten or ligand (e.g., biotin) labeled antibody can be detected by adding an antibody to the hapten or a protein (e.g., avidin) which binds the ligand, tagged with a detectable molecule. Such detectable molecules can be some molecule with a measurable physical property (e.g., fluorescence or absorbance) or a participant in an enzyme reaction (e.g., supra). For example, one can use an enzyme which acts upon a substrate to generate a product with a measurable physical property. Examples of the latter include, but are not limited to, β-galactosidase, alkaline phosphatase and peroxidase. Other labeling schemes will be evident to one of ordinary skill in the art.

Alternatively, the detection antibody reagent can be detected based on a native property such as its own antigenicity. A labeled anti-(antibody) antibody will bind to the primary antibody reagent where the label for the second antibody is a conventional label as above. Further, antibody can be detected by complement fixation or the use of labeled protein A, protein G, as well as other techniques known in the art for detecting antibodies.

Where the detection antibody reagent is labeled, as is preferred, the labeling moiety and the antibody reagent are associated or linked to one another by direct chemical linkage such as involving covalent bonds, or by indirect linkage such as by incorporation of the label in a microcapsule or liposome which is in turn linked to the antibody. Labeling techniques are well-known in the art and any convenient method can be used in the present invention.

The present invention also contemplates the use of kits to detect bioterrorism target agents. The kits may include capture-associated universal oligos and immobilized binding partners to the capture moieties. The kit also may include a universal oligo chip comprising a plurality of chip-associated universal oligos. In preferred embodiments, the kit includes a primer for linear amplification of the capture-associated universal oligo and a T7 or other polymerase in an appropriate buffer. In addition, the kit can include a label for fluorescent detection, an antibody for, e.g., the detection of RNA:RNA or DNA:RNA hybrids, or an electrochemical hybridization indicator for electrochemical detection.

### Example I

### Preparation of Monoclonal Antibodies

A peptide corresponding to amino acid residues in a desired bioterrorism target agent is synthesized with a peptide synthesizer (Applied Biosystems) according to methods known in the art. The peptide emulsified with Freund's complete adjuvant is used as an immunogen. And administered to mice by footpad injection for primary immunization (day 0). The booster immunization is performed four times or more in total. The final immunization is carried out by the same procedure two days before the collection of lymph node cells. The lymph node cells collected from each immunized mouse and mouse myeloma cells are mixed at a ratio of 5:1. Hybridomas are prepared by cell fusion using polyethylene glycol 4000 or polyethylene glycol 1500 (GIBCO) as a fusing agent. The lymph node cells of the mouse are fused with mouse myeloma PAI cells (JCR No. B0113; Res. Disclosure Vol. 217, p. 155, 1982), and the resulting hybridomas are selected by culturing the fused cells in an ASF104 medium (Ajinomoto Co. Inc.) containing HAT supplemented with 10% fetal calf serum (FCS) and aminopterin. The reactivity of the culture supernatant of each hybridoma clone is measured by ELISA.

Screening by ELISA is performed by adding the antibody against the bioterrorism target agent into each well of a 96-well ELISA microplate (Corning Costar Co.). The plate is incubated at room temperature for 2 hours for the adsorption of the antibody against the bioterrorism target agent onto the microplate. The supernatants are discarded and then the blocking reagent (200 μl; phosphate buffer containing 3% BSA) is added into each well. The plate is incubated at room temperature for 0.2 hours to block free sites on the microplate. Each well is washed three times with 200 μl of phosphate buffer containing 0.1% Tween 20. Supernatant (100 μl) from each hybridoma culture is added into each well of the plate, and the reaction is allowed to proceed for 40 minutes. Each well is then washed three times with 200 μl of phosphate buffer containing 0.1% Tween 20. In the next step, biotin-labeled sheep anti-mouse immunoglobulin antibody (50 μl; Amersham) is added to the wells and the plates are incubated at room temperature for 1 hour.

The microplate is washed with phosphate buffer containing 0.1% Tween 20. A solution of strepavidin-β-galactosidase (50 μl; Gibco-BRL), diluted 1000 times with a solution (pH 7.0) containing 20 mM HEPES, 0.5M NaCl and bovine serum albumin (BSA, 1 mg/ml), is added into each well. The plate is then incubated at room temperature for 30 minutes. The microplate is then washed with phosphate buffer containing 0.1% Tween 20. A solution of 1% 4-Methyl-umbelliferyl-β-D-galactoside (50 μl; Sigma) in a phosphate buffer (pH 7.0) containing 100 mM NaCl, 1 mM MgCl₂ and 1 mg/ml BSA, is added into each well. The plate is incubated at room temperature for 10 minutes. 1M Na₂CO₃ (100 μl) is added into each well to stop the reaction. Fluorescence intensity is measured in a Fluoroscan II Microplate Fluorometer (Flow Laboratories Inc.) at a wavelength of 460 nm (excitation wavelength: 355 nm).

### Example II

### Preparation of DNA-Antibody Conjugates

A capture-associated universal oligonucleotide can be prepared on a solid support that has been treated with 3-amino-1,2-propanediol in order to introduce the 3′ amino group with an automated DNA synthesizer (e.g., 3400 DNA synthesizer, Applied Biosystems). Typical cleavage and purification steps are employed to obtain the modified universal oligonucleotide. The universal oligonucleotide is then incubated with N-succinimidyl 3-(2-pyridyldithio)propionate in PBS at a molar ratio between 1:30 to 1:35 for 30 minutes at room temperature. Dithiothreitol is typically added to this solution, resulting in a final concentration of 10 mM for 5 minutes. The universal oligonucleotide is then purified and recovered by applying this reaction mixture to a PBS equilibrated Sepharose column, washing the column several times, and eluting the universal oligonucleotide in a 0.6M NaCl phosphate buffer.

A monoclonal antibody is incubated with γ-maleimidobutyric acid-N-hydroxysuccinimide ester in PBS at a molar ratio of between 1:15 and 1:20 for 30 minutes at room temperature. The maleimide derivatized antibody can then be purified by column chromatography.

The conjugation of the monoclonal antibody and the oligonucleotide is typically achieved by mixing the maleimide derivatized antibody and the sulphydryl containing oligonucleotide in a molar ratio between 1:10 and 1:16 and incubated overnight at 4° C. The resulting conjugates are purified by precipitation with a 50% saturated solution of (NH₄)₂SO₄ and extensive washing in the same (NH₄)₂SO₄ solution. Residual (NH₄)₂SO₄ can then be removed by dissolving the precipitate in PBS and gel filtration.

### Example III

### Immobilization of Nucleic Acid Probe to a Platinum Electrode Surface

A platinum electrode is exposed to a high temperature to air-oxidize the surface of the electrode. The oxidized electrode is treated with cyanogen bromide (CNBr) to activate the oxide layer. The nucleic acid is attached to the electrode by contacting the electrode in a solution of single stranded nucleic acid. The single stranded nucleic acid is obtained by commonly employed means including, but not limited to, either standard oligonucleotide synthesis techniques or by thermal denaturation of a double stranded nucleic acid molecule.

Alternatively, a custom synthesized oligonucleotide containing a thiol group at the 5′ or the 3′ end is spotted on a gold electrode. This procedure involves placing approximately 100 mL of the probe solution containing the oligonucleotide probe (5 μmol/L), 400 mmol/L sodium chloride, and 0.1 mmol/L HCl, on the electrode and then keeping the electrode at room temperature for 1 h thereby resulting in the probes be immobilized onto the gold surface via a thiol moiety. Unattached probes are removed by washing the electrode with distilled water.

### Example IV

### Binding of Bioterrorism Agent Antigen and Removal of Excess Conjugate

A sample is obtained from a patient suspected of having been exposed to or infected by a biological or chemical weapon compound is diluted in PBS/Tween20. An oligonucleotide conjugated to an anti-bioterrorism agent antibody (the procedure for conjugation is described in Example II) is contacted with the diluted sample by adding a one-third volume of bovine serum albumin (12% [wt/vol] in PBS) and 2 μg of antibody-nucleic acid conjugate. The resulting reaction is incubated at room temperature for 30 minutes.

Unbound antibody-nucleic acid conjugate is removed by magnetic microparticle depletion. Briefly, magnetic microparticles are coated with the epitope recognized by the bioterrorism agent antibody. The epitope-coated magnetic beads are added to the reaction mixture, in a PBS buffer supplemented with 0.5% BSA and 2 mM EDTA, and incubated at 4° C. for 30 minutes. Only those antibody-nucleic acid conjugates that have not bound to bioterrorism agent antigen in the sample are available to bind to the immobilized epitope. The magnetically labeled excess conjugate is separated from the reaction mixture by adding the mixture to a column packed with lattice-type matrix and applying a magnetic field. Such separation devices are known in the art (e.g., MACS® Columns, Miltenyi Biotec). The magnetically labeled antibody-nucleic acid conjugate is retained on the column; the target bound conjugates passing through the column and is available for detection.

### Example V

### Cleavage of the Antibody from the Nucleic Acid Strand

Following the isolation of the target bound conjugates, it may be desirable in some instances to remove the antibody and the captured bioterrorism agent antigen from the nucleic acid prior to hybridization. This is accomplished by performing a cleavage reaction to cleave the nucleic acid between the portion of the nucleic acid that will hybridize to the electrode immobilized nucleic acid molecule and the conjugated antibody.

An oligonucleotide is synthesized as described in Example II with a “G-G-C-C” sequence between the conjugated antibody and the portion of the oligonucleotide that will hybridize to the electrode immobilized nucleic acid molecule. The restriction endonuclease, HaeIII (New England Biolabs), has been shown to cleave single stranded DNA at this specific sequence (Horiuchi & Zinder, 1975). The cleavage reaction is performed by mixing the HaeIII enzyme with the antibody-nucleic acid conjugate in a buffer containing 10 mM Tris-HCl, 50 mM NaCl, 10 mM MgCl₂, 1 mM dithiothreitol, pH 7.9, and incubating at 37° C. for 30 minutes. The HaeIII enzyme is heat inactivated at 80° C. for 20 minutes. The cleaved nucleic acid molecules are separated from the antibody-antigen complex by standard techniques such as ethanol precipitation. Briefly, add 2.5-3 volumes of 95% ethanol/0.12 M sodium acetate to the DNA sample contained in a 1.5 ml microcentrifuge tube, invert to mix, and incubate in an ice-water bath for 10 minutes. The resulting mixture is centrifuged at 12,000 rpm in a microcentrifuge for 15 min at 4° C., decant the supernatant, and drain inverted on a paper towel. Ethanol (80%) (corresponding to about two volume of the original sample) is added and the reaction mixture is incubated at room temperature for 5-10 min followed by centrifugation for 5 min. The supernatant is then decanted. The sample is air dried (or alternatively lyophilized) and the pellet of DNA resuspended in 10 mM Tris-HCl, pH 7.6-8.0, 0.1 mM EDTA. For hybridization reactions, the nucleic acid is resuspended in SSC solution.

In an alternative cleavage method, photocleavage is performed. In doing so, an oligonucleotide is synthesized as described in Example II with a photocleavable nucleotide inserted into the sequence. This can be accomplished by using a photocleavable phosphoramidite during the synthesis of the oligonucleotide (Glen Research). The cleavage reaction is essentially performed by exposing the oligonucleotide-antibody conjugate to a source of ultraviolet (UV) light. The cleaved nucleic acid molecules are separated from the antibody-antigen complex by standard techniques such as ethanol precipitation, membrane filtration, or if the antibody-antigen complex is immobilized, by centrifugation, etc.

### Example VI

### Hybridization of Nucleic Acid Molecules to the Electrode-Immobilized Nucleic Acid Molecules

The hybridization and detection reaction is carried out as follows. Single stranded nucleic acid in 2×SSC solution (300 mmol/L NaCl, 30 mmol/L trisodium citrate) are contacted with the probes immobilized on the electrode. The hybridization reaction is carried out at a temperature that permits specific hybridization of the two nucleic acid molecules. The temperature of the hybridization reaction is performed is determined using the equation for calculating the melting temperature of an oligonucleotide. It is possible to shorten the incubation time of this hybridization reaction by applying 0.1 V to the electrode. Using this procedure it may be possible to shorten the incubation time to 10 minutes.

To enhance detection, an electrochemical hybridization indicator, such as a minor groove binder is added. Briefly, a solution containing 50 mmol/L Hoechst 33258 (WAKO Pure Chemicals Industries, Ltd.) and 100 mmol/L NaCl is added before, during, or after hybridization. If the Hoechst 33258 is added after the hybridization reaction, then a further incubation of 5 minutes may be necessary. The electrochemical analysis is carried out with an electrochemical analyzer (Model BAS-100B) and software from Bioanalytical Systems, Inc. or the Genelyzer System from Toshiba Corporation. The cyclic voltammetry is typically carried out at 300 mV/s and 25° C., and the potential sweep range from −100 to 900 mV.

### Example VII

### Binding of Bioterrorism Agent Antigen and Alternative Method of Removal of Excess Conjugate

A sample is obtained from a patient suspected of having been exposed to or infected by a chemical or biological compound is diluted in PBS/Tween20. An oligonucleotide conjugated to a bioterrorism agent antibody (the procedure for conjugation is described in Example II) is contacted with the diluted sample by adding a one-third volume of bovine serum albumin (12% [wt/vol] in PBS) and 2 μg of antibody-nucleic acid conjugate. The resulting reaction is incubated at room temperature for 30 minutes.

Unbound antibody-nucleic acid conjugate is removed by magnetic microparticle depletion. Briefly, magnetic microparticles are coated with a second bioterrorism agent antibody, specific to another region (epitope) of the same bioterrorism agent antigen to be detected. The second antibody-coated magnetic beads are added to the reaction mixture, in a PBS buffer supplemented with 0.5% BSA and 2 mM EDTA, and incubated at 4° C. for 30 minutes. Only those antibody-nucleic acid conjugates that have bound to bioterrorism agent antigen in the sample are available to bind to the magnetic particle immobilized second bioterrorism agent antibody, specific to another region (epitope) of the same bioterrorism agent antigen to be detected. The magnetically labeled conjugate is separated from the reaction mixture by adding the mixture to a column packed with lattice-type matrix and applying a magnetic field. Such separation devices are known in the art (e.g., MACS® Columns, Miltenyi Biotec). The magnetically labeled second antibody-nucleic acid conjugate that is bound to the bioterrorism agent antigen is retained on the column. The antibody-nucleic acid conjugate that is not bound to the bioterrorism agent antigen will pass through the column.

Subsequently, cleavage of the nucleic acid from the magnetically labeled second antibody-nucleic acid conjugate that is bound to the bioterrorism agent antigen is performed as described in Example V. This cleavage can be achieved by other approaches, described earlier in this invention. The cleavage products are then subjected to electrochemical detection.

### Example VIII

### Binding of Bioterrorism Agent Antigen without Direct Interaction with the Causative Agent

A sample is obtained from a patient suspected of having been exposed to or infected by a chemical or biological compound. The sample is diluted in a diluent such as PBS/tween20. An oligonucleotide conjugated to a bioterrorism agent-specific antigen is incubated with the diluted sample by adding a one third volume of bovine serum albumin (12% [wt/vol] in PBS) and 2 us of the oligo nucleotide-antigen conjugate. Unbound nucleic acid-antigen complex is removed by magnetic microparticle-antibody affinity depletion. Briefly, magnetic micro-particles are coated with an antibody affinity reagent such as Protein A, Protein G or anti-class antibody which captures antibodies from the sample, a portion of which may be bioterrorism agent antigen specific and bound to the antigen-oligo conjugate. The coated magnetic beads are added to the reaction mixture, in a PBS buffer supplemented with 0.5% BSA and 2 mm EDTA, and incubated at 40 C for 30 minutes. Antibodies in the sample will be immobilized on the magnetic beads, only anti-bioterrorism agent antibodies will contain the oligo-antigen conjugate. The magnetically labeled antibody affinity reagent, along with additional binding partners (oligo-antigen complexes) are separated from the rest of the sample and extensively washed with PBS/Tween20. Such separation techniques are known in the art (e.g., MACS Columns, Miltenyi Biotec). Subsequent release of the oligo from the antigen is performed as described in Example V or by other approaches described herein.

### Example IX

### Preparation of Loaded Scaffolds Using Gold Particles for the Scaffold Substrate and Antibodies as the Capture Moiety

Loaded scaffolds are created by attaching oligonucleotides and capture moieties onto a substrate. In one example, the scaffold substrate is comprised of gold particles, and the capture moiety is comprised of antibodies. 100 ml of commercially available 0.01% gold chloride solution (e.g., Ted Pella Inc., Redding, Calif.) is adjusted to pH 9.0. Antibody solution is prepared by making a 0.1 μg/μl solution of antibody in 2 mM borax and dialyzing it for at least 4 hours in 1 liter of borax at pH 9.0. The antibody solution is centrifuged at 100,000 g for 1 hour at 4° C. immediately prior to use. The dialyzed and centrifuged antibody solution (0.1 μg/μl) is then adjusted to pH 9.2 using 100 nm K₂CO₃ or 100 mM HCl. An appropriate amount of antibody solution is then added dropwise to 100 ml of the gold pH 9.0 gold solution while stirring rapidly. After 5 minutes, 5 ml of filtered 10% BSA at pH 9.0 is added to this antibody-gold particle solution and stirred gently for 10 minutes. This solution is then purified through centrifugation at approximately 15,000 g for 1 hour at 4° C. for a 15 nm gold particle. Larger gold particles may require a lower centrifugation speed, and smaller gold particles may require a higher centrifugation speed. The gold particle—antibody conjugate will form a loose precipitate at the bottom of the tube. The supernatant is discarded and the pellet is resuspended in a Tris/HCl buffered saline at pH 8.2 with 1% bovine serum albumin and 0.1% sodium azide. The centrifugation at approximately 15,000 g for 1 hour at 4° C. is repeated. Then, 2 ml fractions are carefully pipetted out and examined under electron microscope. The clusters containing the antibody-gold particle scaffold conjugate will be found in the lower fractions.

Oligonucleotides are attached to the antibody-gold particle scaffold through the use of the functionalized chemical group alkylthiol, attached to either terminal end of the oligonucleotide. Alkylthiol, functionalized oligonucleotides are reacted with an appropriate amount of antibody-gold particle scaffold solution for 16 hours and then stabilized with salt to 0.1M NaCl. 10% bovine serum albumin is then added to the solution for 30 minutes to stabilize the gold particle scaffolds. This solution is then purified via centrifugation at 20,000 g for one hour at 4° C., the supernatant is removed, and the centrifugation is repeated. 0.1 M NaCl/0.01M phosphate buffer solution at pH 7.4 is used to resuspend the pellet. The loaded scaffold in the solution comprises antibodies and oligonucleotides associated with a gold particle scaffold.

### Example X

### Preparation of Magnetic Beads with Antibodies Immobilized on the Bead Surface

Magnetic particles (“beads”) may be used as the substrate and antibodies may be attached to form the immobilized binding partner. The use of magnetic beads is well known in the art and are commercially available from such sources as Ademtech Inc., (New York, N.Y.) and Promega U.S. (Madison, Wis.). “Amino-Adembeads” were obtained from Ademtech and these beads consist of a magnetic core encapsulated by a hydrophilic polymer shell, along with a surface activated with amine functionality to assist with immobilization of antibodies to the bead surface. The beads are first washed by placing the beads, in the included “Amino 1 Activation Buffer”, then placing this reaction tube in a magnetic device designed for separation. The supernatant is removed, the reaction tube is removed from the magnet, and the beads are resuspended in the included “Amino 1 Activation Buffer.” To assist coupling of the antibody with the magnetic bead, EDC (1-ethyl-3-(3-dimethlaminopropyl) carbodiimide hydrochloride) (4 mg/ml) is dissolved into the included “Amino 1 Activation Buffer”, and an appropriate amount of this solution is added to the beads (80 μl/mg beads), and vortexed gently. 10-50 mg of antibodies is then added per mg of beads, and the solution is vortexed gently. The solution is incubated for 1 to 2 hours at 37° C. under shaking. Bovine serum albumin (BSA) is then dissolved in “Amino 1 Activation Buffer” to a final concentration of 0.5 mg/ml, and 100 ml of this BSA solution is added to 1 mg of antibody-coated beads, and the solution is vortexed gently and incubated for 30 minutes ant 37° C. under shaking. The beads are then washed in the included “Storage Buffer” twice, and the beads are resuspended.

### Example XI

### Binding of Target Agent and Removal of Excess Unreacted Loaded Scaffold

A sample is obtained from a patient suspected of being infected with or exposed to a chemical or biological compound is diluted in PBS/Tween20. A scaffold conjugated to an anti-bioterrorism agent antibody to form loaded scaffold (the procedure for making such loaded scaffold is described in Example IX) is contacted with the diluted sample by adding a one-third volume of bovine serum albumin (12% [wt/vol] in PBS) and 2 μg of loaded scaffold. The resulting reaction is incubated at room temperature for 60 minutes.

Unbound loaded scaffold is removed by magnetic microparticle depletion. Briefly, magnetic microparticles are coated with the epitope recognized by the bioterrorism agent antigen. The epitope-coated magnetic beads are added to the reaction mixture, in a PBS Buffer supplemented with 0.5% BSA and 2 mM EDTA, and incubated at 4° C. for 30 minutes. Only those loaded scaffolds that have not bound to a bioterrorism agent antigen in the sample (unreacted loaded scaffolds) are available to bind to the immobilized epitope. The magnetically labeled unreacted loaded scaffolds are separated from the reaction mixture by adding the mixture to a column packed with lattice-type matrix and applying a magnetic field. Such separation devices are known in the art (e.g., MACS® Columns, Miltenyi Biotec). The magnetically labeled unreacted loaded scaffolds retained on the column; the reacted loaded scaffolds passing through the column and are available for detection.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. §112, ¶6.

Each reference cited herein is incorporated by reference in its entirety.

1. A method of determining a presence of one or more bioterrorism target agents in a sample comprising:
(a) mixing said sample with capture-associated oligos conjugated to capture moieties specific for said bioterrorism target agents, thereby producing a first mixture comprising reacted capture-associated oligo complexes that are associated with said bioterrorism target agents and unreacted capture-associated oligo complexes that are not associated with said bioterrorism target agents;
(b) contacting said first mixture with immobilized binding partners, wherein said immobilized binding partners facilitate separation of said unreacted capture-associated oligo complexes from said reacted capture-associated oligo complexes to produce a second mixture comprising said unreacted capture-associated oligo complexes and a third mixture comprising said reacted capture-associated oligo complexes;
(c) providing a detection device comprising oligos complementary to said capture-associated oligos, wherein said detection device produces a signal if there is a hybridization event between said capture-associated oligos and said oligos complementary to said capture-associated oligos;
(d) introducing said third mixture to said detection device; and
(e) detecting said signal, wherein said signal is indicative of said presence of said bioterrorism target agents in said sample.

2. A method of detecting the presence of one or more bioterrorism target agents in a sample, said method comprising:
a) forming a first complex by mixing said sample with capture-associated oligo(s), wherein each capture-associated oligo comprises a capture moiety specific for the bioterrorism target agents to be detected, an amplification moiety to enable amplification, and a sequence that is the same or substantially identical to a chip-associated oligo;
b) isolating said first complex from the surrounding solution;
c) amplification of at least part of the capture-associated oligo(s) to form polymerization products with a sequence the same as or substantially identical to the chip-associated oligo(s);
d) contacting the polymerization products with chip-associated oligo(s) to allow hybridization;
e) detection of the hybridization,
wherein detection of the hybridization indicates that one or more bioterrorism target agents was present in the sample.

3. The method of claim 2, wherein said amplification moiety is a promoter.

4. The method of claim 2, wherein said amplification moiety is a PCR primer site.

5. The method of claim 2, wherein said isolating first said complex from the surrounding solution occurs by use of immobilized binding partners.

6. The method of claim 2, wherein said polymerization products are RNA sequences.

7. The method of claim 2, wherein said detection occurs by electrochemical detection.

8. The method of claim 7, wherein said electrochemical detection comprises the use of one or more hybridization indicators.

9. The method of claim 8, wherein the one or more hybridization indicators is selected from the group consisting of: intercalating agents, minor groove binding agents, conjugated antibodies and other nucleic acid binding agents.

10. The method of claim 8, wherein the two or more hybridization indicators used are identical.

11. The method of claim 8, wherein the two or more hybridization indicators used are different from one another.

12. The method of claim 2, wherein said amplification is isothermal amplification.

13. The method of claim 2, wherein said capture-associated oligo(s) encodes a sequence at its 3′ end that is complementary or substantially complementary to a polymerase recognition sequence.

14. The method of claim 2, said method comprising more than one type of capture-associated oligo(s).

15. A method of detecting the presence of one or more bioterrorism target agents in a sample, said method comprising:
a) forming a first complex by mixing said sample with capture-associated oligo(s), wherein each capture-associated oligo comprises:
i) a capture moiety specific for the bioterrorism target agents to be detected;
ii) an amplification moiety to enable amplification;
iii) a sequence at its 3′ end complementary or substantially complementary to a polymerase recognition sequence; and,
iv) a sequence that is the same or substantially identical to a chip-associated oligo;
b) isolating said first complex from the surrounding solution;
c) contacting the first complex with a priming oligonucleotide that is complementary or substantially complementary to the 5′ to 3′ polymerase recognition sequence to form a double-stranded polymerase recognition site;
d) addition of an excess of mononucleotides and polymerase(s);
e) at least one round of amplification of at least part of the capture-associated oligo(s) to form polymerization products with a sequence the same as or substantially identical to the chip-associated oligo(s);
f) contacting the polymerization products with the chip-associated oligos to allow hybridization;
g) detection of the hybridization, wherein detection of the hybridization indicates that one or more bioterrorism target agents was present in the sample.

16. The method of claim 15, wherein said polymerase recognition site is a phage-encoded RNA polymerase recognition site.

17. The method of claim 15, said method comprising more than one type of capture-associated oligo(s).

18. The method of claim 15, wherein hybridization is detected by use of antibody reagents capable of binding to DNA:RNA or RNA: RNA duplexes.

19. The method of claim 18, wherein said antibody reagent is labeled with a moiety.

20. The method of claim 19, wherein said moiety is selected from the group consisting of enzymatically active group, fluorescer, chromophore, luminescer, specifically bindable ligand, electrochemically detectable molecule, and radioisotope.

21. A method of detecting the presence of one or more bioterrorism target agents in a sample, said method comprising:
a) forming a first complex by mixing said sample with capture-associated oligo(s), wherein each capture-associated oligo comprises:
i) a capture moiety specific for the bioterrorism target agents to be detected;
ii) an amplification moiety to enable amplification; and,
iii) a sequence that is the same or substantially identical to a chip-associated oligo;
b) contacting the first complex with an immobilized binding partner to the bioterrorism target agents, thereby forming a second mixture comprising a solution phase and an immobilized phase, wherein the immobilized phase comprises a capture oligo-bioterrorism target agent-immobilized binding partner complex;
c) isolating the immobilized phase from the solution phase and discarding the solution phase;
d) releasing the immobilized phase into a second solution;
e) transferring the second solution to a detection device; and
f) detecting the immobilized phase'
wherein detection of the immobilized phase indicates that one or more bioterrorism target agents was present in the sample.

22. The method of claim 21, further comprising the step of releasing the oligo from the capture oligo-bioterrorism target agent-immobilized binding partner complex.

23. The method of claim 21, further comprising the steps of
a) amplification of at least part of the capture-associated oligo(s) to form polymerization products with a sequence the same as or substantially identical to the chip-associated oligo(s);
b) contacting the polymerization products with chip-associated oligo(s) to allow hybridization;
c) detection of the hybridization,
wherein detection of the hybridization indicates that one or more bioterrorism target agents was present in the sample.
